SEQUENCE LISTING (60 sequences)

SEQ ID NO: 1 - DNA, 122 bp, Artificial Sequence
  Synthesized sequence
aagattgtgtgtggatcgatgatgacttccatatatacattccttggaaagctgaacaaaatgagtgaaaactctataccgtcattctcgtcgaactgaggtccagcacattactccaacag

SEQ ID NO: 2 - DNA, 116 bp, Artificial Sequence
  Synthesized sequence
gagtgagatcttggaccaatgatgacttccatacatgcattccttggaaagctgaacaaaatgagtgggaactctgtactatcatcttagttgaactgaggtccaccgggggctaa

SEQ ID NO: 3 - DNA, 508 bp, Artificial Sequence
  Synthesized sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 4 - RNA, 111 nt, Artificial Sequence
  Synthesized sequence
gggagcagccucuggcauucugggagcuucaucuggaccugggucuucagugaaccauuguucaauaucguccggggacagcaucaaaucauccauugcuugggacggcaa

SEQ ID NO: 5 - RNA, 111 nt, Artificial Sequence
  Synthesized sequence
gacgcccaccgugugguugcuguccaggacggucccggccugcgacacuucggcccagagcugcuccucaucugcggggcgggggggggccgucgccgcguggggucguug

SEQ ID NO: 6 - RNA, 111 nt, Artificial Sequence
  Synthesized sequence
cauauuacagaauaccuugauagcauccaauuugcauccuugguuagggucaacccaguauucuccacucuugaguucaggauggcagaauuucaggucucugcaguuucu

SEQ ID NO: 7 - RNA, 111 nt, Artificial Sequence
  Synthesized sequence
gugaagauaagccaguccucuaguaacagaaugagcaagacggcaagagcuuacccagucacuuguguggagacuuaaauacuugcauaaagauccauugggauaguacuc

SEQ ID NO: 8 - RNA, 111 nt, Artificial Sequence
  Synthesized sequence
gugaacgucaaacugucggaccaauauggcagaaucuucucucaucucaacuuuccauauccguaucauggaaucauagcauccuguaacuacuagcucucuuacagcugg

SEQ ID NO: 9 - RNA, 111 nt, Artificial Sequence
  Synthesized sequence
gccaaugaucucgugaguuaucucagcagugugagccaucagggugaugacaucccaggcgaucguguggccuccaggagcccagagcaggaaguugaggagaaggugccu

SEQ ID NO: 10 - RNA, 111 nt, Artificial Sequence
  Synthesized sequence
caagacggugaaccacuccauggucuucuugucggcuuucugcacuguguacccccagagcuccguguugccgacauccugggguggcuuccacuccagagccacauuaag

SEQ ID NO: 11 - RNA, 111 nt, Artificial Sequence
  Synthesized sequence
aggauucucuuuugaaguauugcucccccaguggauuggguggcuccauucacuccaaugcugagcacuuccacagaguggguuaaagcggcuccgaacacgaaacgugua

SEQ ID NO: 12 - DNA, 31 bp, Artificial Sequence
  Synthesized sequence
tataactagtatggtgagcaagggcgaggag

SEQ ID NO: 13 - DNA, 41 bp, Artificial Sequence
  Synthesized sequence
tatacgtctcatctacagattcttccggcgtgtataccttc

SEQ ID NO: 14 - DNA, 53 bp, Artificial Sequence
  Synthesized sequence
tatacgtctcatagagatccccggtcgccaccgtgagcaagggcgaggagctg

SEQ ID NO: 15 - RNA, 51 nt, Artificial Sequence
  Synthesized sequence
uaaaccgagggaucauaggggacugaauccaccauucuucucccaaucccu

SEQ ID NO: 16 - RNA, 151 nt, Artificial Sequence
  Synthesized sequence
acuacaguugcuccgauauuuaggcuacgucaauaggcacuaacuuauuggcgcuggugaacggacuuccucucgaguaccagaagaugacuacaaaacuccuuuccauugcgaguaucggagucuggcucaguuuggccagggaggcacu

SEQ ID NO: 17 - DNA, 51 bp, Artificial Sequence
  Synthesized sequence
gcccttgctcactggcagagccctccagcatcgcgagcaggcgctgcctcc

SEQ ID NO: 18 - RNA, 151 nt, Artificial Sequence; identical to SEQ ID NO: 16
  Synthesized sequence

SEQ ID NO: 19 - DNA, 116 bp, Artificial Sequence; identical to SEQ ID NO: 2
  Synthesized sequence

SEQ ID NO: 20 - DNA, 122 bp, Artificial Sequence; identical to SEQ ID NO: 1
  Synthesized sequence

SEQ ID NO: 21 - DNA, 708 bp, Artificial Sequence
  Synthesized sequence
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 22 - DNA, 99 bp, Artificial Sequence
  Synthesized sequence
ctgcagggcggaggaggcagcggcggaggaggcagcggcggaggaggcagcagaaggtatacacgccggaagaatctgtagagatccccggtcgccacc

SEQ ID NO: 23 - DNA, 717 bp, Artificial Sequence
  Synthesized sequence
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 24 - DNA, 51 bp, Artificial Sequence
  Synthesized sequence
ctgcagggcggaggaggcagcgcctgctcgcgatgctagagggctctgcca

SEQ ID NO: 25 - RNA, 21 nt, Artificial Sequence
  Synthesized sequence
aaccaugccgacugauggcag

SEQ ID NO: 26 - RNA, 26 nt, Artificial Sequence
  Synthesized sequence
cugccaucagucggcguggacuguag

SEQ ID NO: 27 - RNA, 71 nt, Artificial Sequence
  Synthesized sequence
acagcuccucgcccuugcucacuggcagagcccuccagcaucgcgagcaggcgcugccuccuccgccgcug

SEQ ID NO: 28 - RNA, 121 nt, Artificial Sequence
  Synthesized sequence
cacacacaaacacacacaacacaacacagcuccucgcccuugcucacuggcagagcccuccagcaucgcgagcaggcgcugccuccuccgccgcugcaacacacaaaaccaccacaccaac

SEQ ID NO: 29 - RNA, 171 nt, Artificial Sequence
  Synthesized sequence
accacacacacaaccaccacacacacacacacaaacacacacaacacaacacagcuccucgcccuugcucacuggcagagcccuccagcaucgcgagcaggcgcugccuccuccgccgcugcaacacacaaaaccaccacaccaaccccaacaaccacacacccacacaac

SEQ ID NO: 30 - RNA, 111 nt, Artificial Sequence
  Synthesized sequence
gaugggcaccaccccggugaacagcuccucgcccuugcucacuggcagagcccuccagcaucgcgagcaggcgcugccuccuccgccgcugccuccuccgccgcugccucc

SEQ ID NO: 31 - RNA, 161 nt, Artificial Sequence
  Synthesized sequence
cacacacaaacacacacaacacaacgaugggcaccaccccggugaacagcuccucgcccuugcucacuggcagagcccuccagcaucgcgagcaggcgcugccuccuccgccgcugccuccuccgccgcugccucccaacacacaaaaccaccacaccaac

SEQ ID NO: 32 - RNA, 211 nt, Artificial Sequence
  Synthesized sequence
accacacacacaaccaccacacacacacacacaaacacacacaacacaacgaugggcaccaccccggugaacagcuccucgcccuugcucacuggcagagcccuccagcaucgcgagcaggcgcugccuccuccgccgcugccuccuccgccgcugccucccaacacacaaaaccaccacaccaaccccaacaaccacacacccacacaac

SEQ ID NO: 33 - RNA, 123 nt, Artificial Sequence
  Synthesized sequence
uugccauguguauguggggagacggucggguccagauauucguaucugucgaguagagugugggcuccccacauacucugaugauccagagacgauauuacgucucaggaucauucauggcaa

SEQ ID NO: 34 - RNA, 71 nt, Artificial Sequence; identical to SEQ ID NO: 27
  Synthesized sequence

SEQ ID NO: 35 - RNA, 71 nt, Artificial Sequence
  Synthesized sequence
uuucagcuauaccugcccgguauaaagggacguucacaccgcgauguucucugcuggggaauugcgcgaua

SEQ ID NO: 36 - RNA, 111 nt, Artificial Sequence; identical to SEQ ID NO: 30
  Synthesized sequence

SEQ ID NO: 37 - RNA, 111 nt, Artificial Sequence
  Synthesized sequence
uaccgcuacagccacgcugauuucagcuauaccugcccgguauaaagggacguucacaccgcgauguucucugcuggggaauugcgcgauauucaggauuaaaagaagugc

SEQ ID NO: 38 - RNA, 151 nt, Artificial Sequence; identical to SEQ ID NO: 16
  Synthesized sequence

SEQ ID NO: 39 - RNA, 31 nt, Artificial Sequence
  Synthesized sequence
acuggcagagcccuccagcaucgcgagcagg

SEQ ID NO: 40 - RNA, 51 nt, Artificial Sequence
  Synthesized sequence
gcccuugcucacuggcagagcccuccagcaucgcgagcaggcgcugccucc

SEQ ID NO: 41 - RNA, 71 nt, Artificial Sequence
  Synthesized sequence
acagcuccucgcccuugcucacuggcagagcccuccagcaucgcgagcaggcgcugccuccuccgcccugc

SEQ ID NO: 42 - RNA, 91 nt, Artificial Sequence
  Synthesized sequence
accccggugaacagcuccucgcccuugcucacuggcagagcccuccagcaucgcgagcaggcgcugccuccuccgcccugcagcuuguaca

SEQ ID NO: 43 - RNA, 111 nt, Artificial Sequence
  Synthesized sequence
gaugggcaccaccccggugaacagcuccucgcccuugcucacuggcagagcccuccagcaucgcgagcaggcgcugccuccuccgcccugcagcuuguacagcucguccau

SEQ ID NO: 44 - RNA, 131 nt, Artificial Sequence
  Synthesized sequence
gcucgaccaggaugggcaccaccccggugaacagcuccucgcccuugcucacuggcagagcccuccagcaucgcgagcaggcgcugccuccuccgcccugcagcuuguacagcucguccaugccgccggug

SEQ ID NO: 45 - RNA, 151 nt, Artificial Sequence
  Synthesized sequence
ucgccguccagcucgaccaggaugggcaccaccccggugaacagcuccucgcccuugcucacuggcagagcccuccagcaucgcgagcaggcgcugccuccuccgcccugcagcuuguacagcucguccaugccgccgguggaguggcggc

SEQ ID NO: 46 - RNA, 111 nt, Artificial Sequence; identical to SEQ ID NO: 30
  Synthesized sequence

SEQ ID NO: 47 - RNA, 111 nt, Artificial Sequence; identical to SEQ ID NO: 37
  Synthesized sequence

SEQ ID NO: 48 - RNA, 111 nt, Artificial Sequence; identical to SEQ ID NO: 30
  Synthesized sequence

SEQ ID NO: 49 - RNA, 111 nt, Artificial Sequence; identical to SEQ ID NO: 37
  Synthesized sequence

SEQ ID NO: 50 - RNA, 74 nt, Artificial Sequence
  Synthesized sequence
gcagcggcggaggaggcagcgccugcucgcgaugcuagagggcucugccagugagcaagggcgaggagcuguuc

SEQ ID NO: 51 - RNA, 87 nt, Artificial Sequence
  Synthesized sequence
gagcuguacaagcugcagggcggaggaggcagcgccugcucgcgaugcuagagggcucugccagugagcaagggcgaggagcuguuc

SEQ ID NO: 52 - DNA, 19 bp, Artificial Sequence
  Synthesized sequence
cgcgatgctagagggctct

SEQ ID NO: 53 - DNA, 19 bp, Artificial Sequence
  Synthesized sequence
cgcgatgctggagggctct

SEQ ID NO: 54 - RNA, 52 nt, Artificial Sequence
  Synthesized sequence
aaaauccguugaccuuaaacggucguguggguucaagucccuccacccccac

SEQ ID NO: 55 - RNA, 16 nt, Artificial Sequence
  Synthesized sequence
gagacgcuacggacuu

SEQ ID NO: 56 - RNA, 131 nt, Artificial Sequence
  Synthesized sequence
aacaauagaugacuuacaacuaaucggaaggugcagagacucgacgggagcuacccuaacgucaagacgaggguaaagagagaguccaauucucaaagccaauaggcaguagcgaaagcugcaagagaaug

SEQ ID NO: 57 - RNA, 116 nt, Artificial Sequence
  Synthesized sequence
aaauaauugagccuuaaagaagaaauucuuuaaguggaugcucucaaacucagggaaaccuaaaucuaguuauagacaaggcaauccugagccaagccgaaguaguaauuaguaag

SEQ ID NO: 58 - RNA, 33 nt, Artificial Sequence
  Synthesized sequence
gggagacccucgaccgucgauuguccacugguc

SEQ ID NO: 59 - RNA, 35 nt, Artificial Sequence
  Synthesized sequence
accaguggacaaucgacggauaacagcauaucuag

SEQ ID NO: 60 - RNA, 19 nt, Artificial Sequence
  Synthesized sequence
uaauacgacucacuauagg

(19) United States
(12) Patent Application Publication
(10) Pub. No.: US 2024/0309368 A1
(43) Pub. Date: Sep. 19, 2024

(54) TARGETED RNA EDITING BY LEVERAGING ENDOGENOUS ADAR USING ENGINEERED RNAS

(71) Applicant: Peking University, Beijing (CN)

(72) Inventors: Wensheng WEI, Beijing (CN); Zongyi YI, Beijing (CN); Liang QU, Beijing (CN)

(21) Appl. No.: 18/272,009

(22) PCT Filed: Jan. 12, 2021
(86) PCT No.: PCT/CN2021/071292

§ 371 Date: Jul. 12, 2023

(51) Int. Cl. C12N 15/113; A61P 3/06; C12N 9/00; C12N 9/80

(52) U.S. Cl. CPC C12N 15/113; A61P 3/06; C12N 9/80; C12N 9/93; C12N 2310/124; C12N 2320/33; C12Y 305/04004; C12Y 605/01003

(57) ABSTRACT

Provided are methods for editing RNA by introducing a deaminase-recruiting RNA in a host cell for deamination of an adenosine in a target RNA. Further provided are deaminase-recruiting RNAs used in the RNA editing methods and compositions and kits comprising the same.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: FE00330PCTR-prj_ST25.txt, date recorded: Jan. 12, 2021, size: 18 KB).

### FIELD

The present disclosure relates generally to methods and compositions for editing RNAs using an engineered RNA capable of recruiting an adenosine deaminase to deaminate one or more adenosines in target RNAs.

### BACKGROUND

Genome editing is a powerful tool for biomedical research and development of therapeutics for diseases. Editing technologies using engineered nucleases, such as zinc finger nucleases (ZFNs), transcription activator-like effector nucleases (TALENs), and Cas proteins of CRISPR system have been applied to manipulate the genome in a myriad of organisms. Recently, taking advantage of the deaminase proteins, such as Adenosine Deaminase Acting on RNA (ADAR), new tools were developed for RNA editing. In mammalian cells, there are three types of ADAR proteins, Adar1 (two isoforms, p110 and p150), Adar2 and Adar3 (catalytically inactive). The catalytic substrate of ADAR protein is double-stranded RNA, and ADAR can remove the —NH2 group from an adenosine (A) nucleobase, changing A to inosine (I). (I) is recognized as guanosine (G) and paired with cytidine (C) during subsequent cellular transcription and translation processes. To achieve targeted RNA editing, the ADAR protein or its catalytic domain was fused with a kN peptide, a SNAP-tag or a Cas protein (dCas13b), and a guide RNA was designed to recruit the chimeric ADAR protein to the target site. Alternatively, overexpressing ADAR1 or ADAR2 proteins together with an R/G motif-bearing guide RNA was also reported to enable targeted RNA editing.

However, currently available ADAR-mediated RNA editing technologies have certain limitations. For example, the most effective in vivo delivery for gene therapy is through viral vectors, but the highly desirable adeno-associated virus (AAV) vectors are limited with the cargo size (˜4.5 kb), making it challenging for accommodating both the protein and the guide RNA. Furthermore, over-expression of ADAR1 has recently been reported to confer oncogenicity in multiple myelomas due to aberrant hyper-editing on RNAs, and to generate substantial global off-targeting edits. In addition, ectopic expression of proteins or their domains of non-human origin has potential risk of eliciting immunogenicity. Moreover, pre-existing adaptive immunity and p53-mediated DNA damage response may compromise the efficacy of the therapeutic protein, such as Cas9.

### BRIEF SUMMARY

The present application provides methods of RNA editing using ADAR-recruiting RNAs (“dRNA” or “arRNA”) which are capable of leveraging endogenous Adenosine Deaminase Acting on RNA (“ADAR”) proteins for the RNA editing. Also provided herein are engineered dRNAs or constructs comprising a nucleic acid encoding the engineered dRNAs used in these methods, and compositions and kits comprising the same. Also provided herein are methods for treating or preventing a disease or condition in an individual comprising editing a target RNA associated with the disease or condition in a cell of the individual.

In one aspect, provided herein are methods for editing a target RNA in a host cell, comprising introducing a deaminase-recruiting RNA (dRNA) or a construct comprising a nucleic acid encoding the dRNA into the host cell, wherein:
(1) the dRNA comprises a targeting RNA sequence that is at least partially complementary to the target RNA,
(2) the dRNA is capable of recruiting an adenosine deaminase acting on RNA (ADAR), and
(3) the dRNA is a circular RNA or capable of forming a circular RNA in the host cell.

In some embodiments, the dRNA is a linear RNA capable of forming a circular RNA in the host cell. In some embodiments, the dRNA further comprises a 3′ ligation sequence and a 5′ ligation sequence. In some embodiments, the 3′ ligation sequence and the 5′ ligation sequence are at least partially complementary to each other. In some embodiments, the 3′ ligation sequence and the 5′ ligation sequence are about 20 to about 75 nucleotides in length. In some embodiments, the dRNA is circularized by an RNA ligase. In some embodiments, the RNA ligase is RNA ligase RtcB. In some embodiments, the RNA ligase RtcB is expressed endogenously in the host cell. In some embodiments, the dRNA is a circular RNA.

In some embodiments, the method comprises introducing a construct comprising a nucleic acid encoding the dRNA into the host cell. In some embodiments, the construct further comprises a 3′ twister ribozyme sequence linked to the 3′ end of the nucleic acid encoding the dRNA and a 5′ twister ribozyme sequence linked to the 5′ end of the nucleic acid encoding the dRNA. In some embodiments, the 3′ twister sequence is twister P3 U2A and the 5′ twister sequence is twister P1. In some embodiments, the 5′ twister sequence is twister P3 U2A and the 3′ twister sequence is twister P1.

In some embodiments, wherein the dRNA is a circular RNA, the method further comprises forming the circular RNA using a linear RNA in vitro.

In some embodiments, the dRNA is a circular RNA formed using a linear RNA in vitro by autocatalysis of a Group I intron comprising a 5′ catalytic Group I intron fragment and a 3′ catalytic Group I intron fragment. In some embodiments, the linear RNA comprises the 3′ catalytic Group I intron fragment flanking the 5′ end of a 3′ exon sequence recognizable by the 3′ catalytic Group I intron fragment, and the 5′ catalytic Group I intron fragment flanking the 3′ end of a 5′ exon sequence recognizable by the 5′ catalytic Group I intron fragment. In some embodiments, the linear RNA further comprises a 5′ homology sequence flanking the 5′ end of the 3′ catalytic Group I intron fragment, and a 3′ homology sequence flanking the 3′ end of the 5′ catalytic Group I intron fragment. In some embodiments, said forming a circular RNA comprises: (a) subjecting the linear RNA to a condition that activates autocatalysis of the 5′ catalytic Group I intron fragment and the 3′ catalytic Group I intron fragment to provide a circularized RNA product; and (b) isolating the circularized RNA product, thereby providing the circular RNA.

In some embodiments, the dRNA is a circular RNA formed using a linear RNA in vitro by a ligase. In some embodiments, the ligase is selected from the group consisting of a T4 DNA ligase (T4 Dnl), a T4 RNA ligase 1 (T4 Rnl1) and a T4 RNA ligase 2 (T4 Rnl2). In some embodiments, the linear RNA comprises a 5′ ligation sequence at the 5′ end of a nucleic acid sequence encoding the circular RNA, and a 3′ ligation sequence at the 3′ end of the nucleic acid sequence encoding the circular RNA, wherein the 5′ ligation sequence and the 3′ ligation sequence can be ligated to each other via the ligase. In some embodiments, said forming a circular RNA comprises: (a) contacting the linear RNA with a single-stranded adaptor nucleic acid comprising from the 5′ end to the 3′ end: a first sequence complementary to the 3′ ligation sequence and a second sequence complementary to the 5′ ligation sequence, and wherein the 5′ ligation sequence and the 3′ ligation sequence hybridize to the single-stranded adaptor nucleic acid to provide a duplex nucleic acid intermediate comprising a single strand break between the 3′ end of the 5′ ligation sequence and the 5′ end of the 3′ ligation sequence; (b) contacting the intermediate with an RNA ligase under a condition that allows ligation of the 5′ ligation sequence to the 3′ ligation sequence to provide a circularized RNA product; and (c) isolating the circularized RNA product, thereby providing the circular RNA. In some embodiments, said forming a circular RNA comprises: (a) contacting the linear RNA with an RNA ligase under a condition that allows ligation of the 5′ ligation sequence to the 3′ ligation sequence to provide a circularized RNA product; and (b) isolating the circularized RNA product, thereby providing the circular RNA.

In some embodiments, wherein the method comprises forming the circular RNA using a linear RNA in vitro, the method further comprises obtaining the linear RNA by in vitro transcription of a nucleic acid construct comprising a nucleic acid sequence encoding the linear RNA. In some embodiments, the method further comprises purifying the circular RNA.

In another aspect, provided herein are methods for editing a target RNA in a host cell, comprising introducing a deaminase-recruiting RNA (dRNA) or a construct comprising a nucleic acid encoding the dRNA into the host cell, wherein the dRNA comprises:
(1) a targeting RNA sequence that is at least partially complementary to the target RNA and
(2) a small nucleolar RNA (snoRNA) sequence linked to the 3′ and/or 5′ ends of the targeting RNA sequence;
   and wherein the dRNA is capable of recruiting an adenosine deaminase acting on RNA (ADAR).

In some embodiments, the dRNA comprises a snoRNA sequence linked to the 5′ end of the targeting RNA sequence (“5′ snoRNA sequence”). In some embodiments, the dRNA comprises a snoRNA sequence linked to the 3′ end of the targeting RNA sequence (3′ snoRNA sequence”). In some embodiments, the snoRNA sequence is at least about 70 nucleotides in length. In some embodiments, the 3′ snoRNA sequence comprises the nucleic acid sequence of SEQ ID NO: 1. In some embodiments, the 5′ snoRNA sequence comprises the nucleic acid sequence of SEQ ID NO: 2. In some embodiments, the snoRNA sequence is a C/D Box snoRNA sequence. In some embodiments, the snoRNA sequence is an H/ACA Box snoRNA sequence. In some embodiments, the snoRNA sequence is a composite C/D Box and H/ACA Box snoRNA sequence. In some embodiments, the snoRNA sequence is an orphan snoRNA sequence. In some embodiments, the method comprises introducing a construct comprising a nucleic acid encoding the dRNA into the host cell. In some embodiments, the construct further comprises a promoter operably linked to the nucleic acid encoding the dRNA. In some embodiments, the promoter is a polymerase II promoter (“Pol II promoter”). In some embodiments, the construct is a viral vector or a plasmid. In some embodiments, the construct is an AAV vector.

In another aspect, provided herein are methods for editing a target RNA in a host cell, comprising introducing a construct comprising a nucleic acid encoding a deaminase-recruiting RNA (dRNA) into the host cell, wherein:
(1) the dRNA comprises a targeting RNA sequence that is at least partially complementary to the target RNA,
(2) the dRNA is capable of recruiting an adenosine deaminase acting on RNA (ADAR), and
(3) the construct comprises a polymerase II promoter (“Pol H promoter”) operably linked the nucleic acid encoding the dRNA.

In some embodiments, the pol II promoter is a CMV promoter. In some embodiments, the CMV promoter comprises the nucleic acid sequence of SEQ ID NO: 3.

The following embodiments are applicable to all three aspects described above. In some embodiments, the construct is a viral vector or a plasmid. In some embodiments, the construct is an AAV vector. In some embodiments, the ADAR is endogenously expressed by the host cell. In some embodiments, the host cell is a T cell. In some embodiments, the targeting RNA sequence is more than 50 nucleotides in length. In some embodiments, the targeting RNA sequence is about 100 to about 180 nucleotides in length. In some embodiments, the targeting RNA sequence is about 100 to about 150 nucleotides in length. In some embodiments, the targeting RNA sequence comprises a cytidine, adenosine or uridine directly opposite the target adenosine in the target RNA. In some embodiments, the targeting RNA sequence comprises a cytidine mismatch directly opposite the target adenosine in the target RNA. In some embodiments, the cytidine mismatch is located at least 20 nucleotides away from the 3′ end of the targeting RNA sequence, and at least 5 nucleotides away from the 5′ end of the targeting RNA sequence. In some embodiments, the targeting RNA sequence further comprises one or more guanosines each opposite a non-target adenosine in the target RNA. In some embodiments, the targeting RNA sequence comprises two or more consecutive mismatch nucleotides opposite a non-target adenosine in the target RNA.

In some embodiments, the 5′ nearest neighbor of the target adenosine in the target RNA is a nucleotide selected from U, C, A and G with the preference U>C≈A>G and the 3′ nearest neighbor of the target adenosine in the target RNA is a nucleotide selected from G, C, A and U with the preference G>C>A≈U. In some embodiments, the target adenosine is in a three-base motif selected from the group consisting of UAG, UAC, UAA, UAU, CAG, CAC, CAA, CALU, AAG, AAC, AAA, AAU, GAG, GAC, GAA and GAU in the target RNA. In some embodiments, the three-base motif is UAG, and the targeting RNA comprises an A directly opposite the uridine in the three-base motif, a cytidine directly opposite the target adenosine, and a cytidine, guanosine or uridine directly opposite the guanosine in the three-base motif. In some embodiments, the target RNA is an RNA selected from the group consisting of a pre-messenger RNA, a messenger RNA, a ribosomal RNA, a transfer RNA, a long non-coding RNA and a small RNA. In some embodiments, the target RNA is a pre-messenger RNA.

In some embodiments, the method further comprises introducing an inhibitor of ADAR3 to the host cell. In some embodiments, the method further comprises introducing a stimulator of interferon to the host cell. In some embodiments, the method comprises introducing a plurality of dRNAs or constructs each targeting a different target RNA. In some embodiments, the method further comprises introducing an ADAR (e.g., exogenous ADAR) to the host cell. In some embodiments, the efficiency of editing the target RNA is at least 40%. In some embodiments, the construct or the dRNA does not induce immune response. In some embodiments, the ADAR is an ADAR1 comprising an E1008 mutation.

In some embodiments, deamination of the target adenosine in the target RNA results in a missense mutation, an early stop codon, aberrant splicing, or alternative splicing in the target RNA, or reversal of a missense mutation, an early stop codon, aberrant splicing, or alternative splicing in the target RNA. In some embodiments, deamination of the target adenosine in the target RNA results in point mutation, truncation, elongation and/or misfolding of the protein encoded by the target RNA, or a functional, full-length, correctly-folded and/or wild-type protein by reversal of a missense mutation, an early stop codon, aberrant splicing, or alternative splicing in the target RNA. In some embodiments, the host cell is a eukaryotic cell. In some embodiments, the host cell is a mammalian cell. In some embodiments, the host cell is a human or mouse cell. Also provided herein are edited RNAs or host cells having the edited RNAs produced by any one of the methods provided in the above three aspects.

In another aspect, provided herein are methods for treating or preventing a disease or condition in an individual, comprising editing a target RNA associated with the disease or condition in a cell of the individual according to any one of the methods provided above. In some embodiments, the disease or condition is a hereditary genetic disease or a disease or condition associated with one or more acquired genetic mutations. In some embodiments, the target RNA has a G to A mutation. In some embodiments, the disease or condition is a monogenetic disease or condition. In some embodiments, the disease or condition is a polygenetic disease or condition.

In some embodiments, the target RNA is TP53, and the disease or condition is cancer. In some embodiments, the target RNA is IDUA, and the disease or condition is Mucopolysaccharidosis type I (MPS I). In some embodiments, the target RNA is COL3A1, and the disease or condition is Ehlers-Danlos syndrome. In some embodiments, the target RNA is BMPR2, and the disease or condition is Joubert syndrome. In some embodiments, the target RNA is FANCC, and the disease or condition is Fanconi anemia. In some embodiments, the target RNA is MYBPC3, and the disease or condition is primary familial hypertrophic cardiomyopathy. In some embodiments, the target RNA is IL2RG, and the disease or condition is X-linked severe combined immunodeficiency.

In another aspect, provided herein is a deaminase-recruiting RNAs (dRNA) for editing a target RNA in a host cell comprising a targeting RNA sequence that is at least partially complementary to the target RNA, wherein the dRNA is capable of recruiting an Adenosine Deaminase Acting on RNA (ADAR), and wherein the dRNA is circular or is capable of forming a circular RNA in the host cell. In some embodiments, the dRNA is a linear RNA capable of forming a circular RNA. In some embodiments, the dRNA further comprises a 3′ ligation sequence and a 5′ ligation sequence. In some embodiments, the 3′ ligation sequence and the 5′ ligation sequence are at least partially complementary to each other. In some embodiments, the 3′ ligation sequence and the 5′ ligation sequence are about 20 to about 75 nucleotides in length.

In some embodiments, the dRNA is a circular RNA. In some embodiments, the dRNA is a circular RNA formed from a linear RNA in vitro. In some embodiments, the dRNA is a linear RNA capable of forming a circular RNA in the host cell. In some embodiments, the dRNA is a circular RNA that is formed by circularizing a linear RNA via autocatalysis of a Group I intron comprising a 5′ catalytic Group I intron fragment and a 3′ catalytic Group I intron fragment. In some embodiments, the linear RNA comprises the 3′ catalytic Group I intron fragment flanking the 5′ end of a 3′ exon sequence recognizable by the 3′ catalytic Group I intron fragment, and the 5′ catalytic Group I intron fragment flanking the 3′ end of a 5′ exon sequence recognizable by the 5′ catalytic Group I intron fragment. In some embodiments, the linear RNA further comprises a 5′ homology sequence flanking the 5′ end of the 3′ catalytic Group I intron fragment, and a 3′ homology sequence flanking the 3′ end of the 5′ catalytic Group I intron fragment. In some embodiments, the linear RNA is circularized by a ligase. In some embodiments, the ligase is selected from the group consisting of a T4 DNA ligase (T4 Dnl), a T4 RNA ligase 1 (T4 Rnl1) and a T4 RNA ligase 2 (T4 Rnl2). In some embodiments, the linear RNA comprises a 5′ ligation sequence at the 5′ end of the nucleic acid sequence encoding the circular dRNA, and a 3′ ligation sequence at the 3′ end of the nucleic acid sequence encoding the circular dRNA, wherein the 5′ ligation sequence and the 3′ ligation sequence can be ligated to each other via the RNA ligase.

In some embodiments, the target RNA is an RNA selected from the group consisting of a pre-messenger RNA, a messenger RNA, a ribosomal RNA, a transfer RNA, a long non-coding RNA and a small RNA.

Also provided is a linear RNA capable of forming the circular dRNA as described in this aspect. Also provided herein are kits for editing a target RNA in a host cell comprising the linear dRNA as described in this aspect.

Also provided herein are constructs comprising a nucleic acid encoding the dRNA as described in this aspect. In some embodiments, the construct further comprises a 3′ twister ribozyme sequence linked to the 3′ end of the nucleic acid encoding the dRNA and a 5′ twister ribozyme sequence linked to the 5′ end of the nucleic acid encoding the dRNA. In some embodiments, the 3′ twister sequence is twister P3 U2A and the 5′ twister sequence is twister P1. In some embodiments, wherein the 5′ twister sequence is twister P3 U2A and the 3′ twister sequence is twister P1. Also provided herein are host cells comprising the construct or dRNA as described in this aspect. Also provided herein are kits for editing a target RNA in a host cell comprising the construct or dRNA as described in this aspect.

In another aspect, also provided herein is a deaminase-recruiting RNA (dRNA) for editing a target RNA comprising:
(1) a targeting RNA sequence that is at least partially complementary to the target RNA and
(2) a small nucleolar RNA (snoRNA) sequence at the 3′ and/or 5′ ends of the targeting RNA sequence;
   wherein the dRNA is capable of recruiting an adenosine deaminase acting on RNA (ADAR).

In some embodiments, the dRNA comprises a snoRNA sequence linked to the 5′ end of the targeting RNA sequence (“5′ snoRNA sequence”). In some embodiments, the dRNA comprises a snoRNA sequence linked to the 3′ end of the targeting RNA sequence (3′ snoRNA sequence”). In some embodiments, the snoRNA sequence is at least about 70 nucleotides in length. In some embodiments, the 3′ snoRNA sequence comprises the nucleic acid sequence of SEQ ID NO: 1. In some embodiments, the 5′ snoRNA sequence comprises the nucleic acid sequence of SEQ ID NO: 2. In some embodiments, the snoRNA sequence is a C/D Box snoRNA sequence. In some embodiments, the snoRNA sequence is an H/ACA Box snoRNA sequence. In some embodiments, the snoRNA sequence is a composite C/D Box and H/ACA Box snoRNA sequence. In some embodiments, the snoRNA sequence is an orphan snoRNA sequence. Also provided herein are constructs comprising a nucleic acid encoding the dRNA as described in this aspect. In some embodiments, the construct further comprises a promoter operably linked to the nucleic acid encoding the dRNA. In some embodiments, the promoter is a polymerase II promoter (“Pol II promoter”). In some embodiments, the target RNA is an RNA selected from the group consisting of a pre-messenger RNA, a messenger RNA, a ribosomal RNA, a transfer RNA, a long non-coding RNA and a small RNA. Also provided herein are host cells comprising the construct or dRNA as described in this aspect. Also provided herein are kits for editing a target RNA in a host cell comprising the construct or dRNA as described in this aspect.

In another aspect, provided herein is a construct comprising a nucleic acid encoding a deaminase-recruiting RNA (dRNA) into the host cell, wherein:
(1) the dRNA comprises a targeting RNA sequence that is at least partially complementary to the target RNA,
(2) the dRNA is capable of recruiting an adenosine deaminase acting on RNA (ADAR), and
(3) the construct comprises a polymerase II promoter (“Pol II promoter”) operably linked to the nucleic acid encoding the dRNA.

In some embodiments, the pol II promoter is a CMV promoter. In some embodiments, the CMV promoter comprises the nucleic acid sequence of SEQ ID NO: 3. In some embodiments, the construct is a viral vector or a plasmid. In some embodiments, the construct is an AAV vector. In some embodiments, the target RNA is an RNA selected from the group consisting of a pre-messenger RNA, a messenger RNA, a ribosomal RNA, a transfer RNA, a long non-coding RNA and a small RNA. Also provided herein are host cells comprising the construct as described in this aspect. Also provided herein are kits for editing a target RNA in a host cell comprising the construct as described in this aspect.

It is to be understood that one, some, or all of the properties of the various embodiments described herein may be combined to form other embodiments of the present application. These and other embodiments of the present application are further described by the detailed description that follows.

### BRIEF DESCRIPTION OF THE DRAWINGS

FIG. 1 depicts FACS analysis after transfection of a plasmid expressing arRNA driven by a pol II promoter (CMV) and a plasmid expressing arRNA driven by a Pol III promoter (U6) for 48 hours. The EGFP positive percentages were normalized by transfection efficiency, which was determined by mCherry positive ratio. Data are mean values±s.d. (n=3).

FIGS. 2A-2C depict Sno-arRNA₁₅₁ flanked by snoRNA ends mediated targeted RNA editing on Reporter mRNA. FIG. 2A shows a schematic of arRNA-expressing plasmid. The 151-nt arRNA targeting fluorescence Reporter-1 was expressed under human U6 promoter. FIG. 2B shows the FACS results of sno-arRNA or arRNA transfection results. The sno-arRNA₁₅₁, arRNA₁₅₁, sno-Ctrl RNA₁₅₁ or Ctrl RNA₁₅₁ was transfected into HEK293T cells along with Reporter-expressing plasmids. FIG. 2C shows quantification of the FACS results in FIG. 2B.

FIGS. 3A-3C depict editing efficacy with CMV-promoter expressed sno-arRNA and hU6 promoter expressed sno-arRNA. FIG. 3A shows the quantificational FACS results of hU6-derived sno-arRNA or arRNA at different time point. FIG. 3B shows the quantificational FACS results of CMV or hU6 derived arRNA at different time point. FIG. 3C shows the quantificational FACS results of CMV or hU6 derived sno-arRNA at different time point.

FIGS. 4A-4E depict editing efficacy using circular arRNA in the LEAPER system. FIG. 4A shows a schematic of circular arRNA expression. Circular arRNA transcript was flanked by 5′ and 3′ ligation sequence, which were respectively flanked by 5′-Twister P3 U2A and 3′-Twister P1 ribozymes undergoing self-cleavage. The resulting RNA ends were recognized by RtcB for ligation. FIG. 4B shows a schematic of the Reporter-1 and Reporter-3. mCherry and EGFP genes were linked by sequences containing 3× (for Reporter-1) or 1× (for Reporter-3) GGGGS-coding region and an in-frame UAG stop codon. The reporter expressed cells only produced mCherry protein, while targeted editing on the UAG stop codon of the reporter transcript could convert the UAG to UIG and thus to permit the downstream EGFP expression. FIG. 4C shows results of an experiment, in which HEK293T cells stably expressing the Reporter-1 seeded in 12-well plates (3×10⁵ cells/well) were transfected with the 1 μg of circular arRNA₇₁, circular arRNA₂₅₋₇₁₋₂₅, circular arRNA₅₀₋₇₁₋₅₀, circular arRNA₁₁₁, circular arRNA₂₅₋₁₁₁₋₂₅, circular arRNA₅₀₋₁₁₁₋₅₀, circular Ctrl RNA₁₂₃ (non-target sequence), arRNA₇₁, Ctrl RNA₇₁, arRNA₁₁₁, Ctrl RNA₁₁₁ expressing plasmid respectively. FACS analyses were performed 2 days and 7 days after transfection. The ratios of EGFP+ cells were normalized by transfection efficiency. FIG. 4D shows results of an experiment, in which HEK293T cells stably expressing the Reporter-3 seeded in 12-well plates (3×10⁵ cells/well) were transfected with the 1 μg of Ctrl RNA₁₅₁ (circular), 31-, 51-, 71-, 91-, 111-, 131-, 151-nt circular arRNA expressing plasmid respectively. FACS analyses were performed 2 days post transfection. The ratios of EGFP+ cells were normalized by transfection efficiency. FIG. 4E shows results of an experiment, in which HeLa and A549 cells seeded in 12-well plates (2×10⁵ cells/well) were co-transfected with the 0.5 μg of reporter-1 expressing plasmid and the 0.5 μg of circular arRNA₁₁₁ expressing plasmid respectively. FACS analyses were performed 2 days post transfection. The ratios of EGFP+ cells were normalized by transfection efficiency.

FIG. 5 depicts FACS analysis after co-transfection of various circular arRNAs for 48 hrs and 96 hrs, and EGFP positive percentages were normalized by transfection efficiency, which was determined by mCherry positive. Data are mean values±s.d. (n=3).

FIGS. 6A-6B depict FACS analysis after co-transfection of various circular arRNAs in HEK293T cells for 48 hrs and 96 hrs. EGFP positive percentages were normalized by transfection efficiency, which was determined by mCherry positive. Data are mean values s.d. (n=3).

FIG. 7 depicts quantification of the EGFP positive (EGFP+) cells. Cells stably expressing Reporter-1 were infected with various Tornado-arRNA lentivirus, including the Tornado Ctrl RNA₁₁₁ virus and the targeting Tornado-arRNA virus with different length, followed by FACS 2 days after infection. Data are mean values±s.e.m. (n=2).

FIG. 8A is a schematic illustrating in vitro production of a circular dRNA (also referred to as arRNA) via ligation of a linear in vitro RNA transcript bound to a single stranded DNA (ssDNA) adaptor using a T4 RNA ligase.

FIG. 8B is a schematic illustrating in vitro production of a circular dRNA (also referred to as arRNA) via ligation of a linear in vitro RNA transcript by a T4 RNA ligase without using a ssDNA adaptor.

FIG. 8C is a schematic illustrating in vitro production of a circular DNA (also referred to as arRNA) via splicing of Group I catalytic Introns.

FIGS. 9A-9C show HPLC chromatograms of a linear precursor RNA (FIG. 9A), T4 Rnl1-treated RNA (FIG. 9B) and T4-Rnl2 treated RNA (FIG. 9C), which were designed to target an EGFP reporter in cells.

FIG. 10 shows fluorescent microscopy images of cells after transfection of a linear precursor RNA, T4 Rnl1-treated RNA, and T4 Rnl2-treated RNA targeting an EGFP reporter for 1 day, 3 days and 7 days.

FIG. 11 shows Sanger sequencing results of the target site in cells transfected with a linear precursor RNA (top), T4 Rnl1-treated RNA (middle) and T4 Rnl2-treated RNA (bottom) targeting an EGFP reporter for 48 hours.

FIG. 12 shows deep sequencing results of the target site in cells transfected with a linear precursor RNA (top), T4 Rnl1-treated RNA (middle) and T4 Rnl2-treated RNA (bottom) targeting an EGFP reporter for 48 hours.

FIG. 13A shows deep sequencing results of the endogenous PPIB target site in cells transfected with control (circ-Ctrl RNA₁₅₁), circ-arRNA₁₅₁-PPIB or U6-arRNA₁₅₁-PPIB construct for 48 hours. The U6-arRNA₁₅₁-PPIB construct is a U6 promoter driven plasmid expressing a dRNA targeting the same PPIB target site.

FIG. 13B shows deep sequencing results of the endogenous IDUA target site in cells after transfected with control (circ-Ctrl RNA₁₅₁) or circ-arRNA₁₅₁-IDUA for 48 hours.

### DETAILED DESCRIPTION

The present description provides RNA editing methods (referred herein as the “improved LEAPER” methods) and specially designed RNAs, referred herein as deaminase-recruiting RNAs (“dRNAs”) or ADAR-recruiting RNAs (“arRNAs”) or constructs comprising nucleic acids encoding these dRNAs, to edit target RNAs in a host cell.

“LEAPER” (Leveraging Endogenous ADAR for Programmable Editing on RNA) have been previously developed by inventors of the present application, which leverages endogenous ADAR to edit target RNA by utilizing dRNAs, also referred to as “arRNAs.” LEAPER method was described in PCT/CN2018/110105 and PCT/CN2020/101248, which are incorporated herein by reference in their entirety. Specifically, a targeting RNA that is partially complementary to the target transcript was used to recruit native ADAR1 or ADAR2 to change adenosine to inosine at a specific site in a target RNA. As such, RNA editing can be achieved in certain systems without ectopic or overexpression of the ADAR proteins in the host cell.

The present application provided improved LEAPER methods that allow for increased efficiency for RNA editing, for example by increasing the level of the dRNA in target cells. In one aspect, the improved LEAPER method involves use of circular dRNA (e.g., circular dRNA formed from a linear RNA in vitro) or dRNA capable of forming a circular RNA in target host cells. In another aspect, the improved LEAPER method involves use of dRNA comprising one or more small nucleolar RNA (snoRNA) linked to the 3′ or 5′ of the targeting RNA sequence. In another aspect, the improved LEAPER method involves dRNAs placed under the control of a polymerase II promoter (“Pol II promoter”). We demonstrated that, compared to LEAPER methods, the improved LEAPER methods significantly increased the editing efficiency of the dRNA. Without being bound by theory, it is believed that an increase in stability or amount of the dRNA used in the improved LEAPER methods contributed to such improvement in RNA editing efficiency.

Thus, the present application in one aspect provides a method of editing a target RNA by one or more of the improved LEAPER methods.

In another aspect, there are provided dRNAs and constructs used for the improved LEAPER methods.

Also provided herein are methods and compositions for treating or preventing a disease or condition in an individual using the RNA editing methods.

### I. DEFINITIONS

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to particular method steps, reagents, or conditions are specifically embraced by the present disclosure and are disclosed herein just as if each and every combination was individually and explicitly disclosed.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The terms “deaminase-recruiting RNA,” “dRNA,” “ADAR-recruiting RNA” and “arRNA” are used herein interchangeably to refer to an engineered RNA capable of recruiting an ADAR to deaminate a target adenosine in an RNA.

The terms “polynucleotide,” “nucleotide sequence” and “nucleic acid” are used interchangeably. They refer to a polymeric form of nucleotides of any length, either deoxyribonucleotides or ribonucleotides, or analogs thereof.

The terms “adenine,” “guanine,” “cytosine,” “thymine,” “uracil” and “hypoxanthine” as used herein refer to the nucleobases as such. The terms “adenosine,” “guanosine,” “cytidine,” “thymidine,” “uridine” and “inosine,” refer to the nucleobases linked to the ribose or deoxyribose sugar moiety. The term “nucleoside” refers to the nucleobase linked to the ribose or deoxyribose. The term “nucleotide” refers to the respective nucleobase-ribosyl-phosphate or nucleobase-deoxyribosyl-phosphate. Sometimes the terms adenosine and adenine (with the abbreviation, “A”), guanosine and guanine (with the abbreviation, “G”), cytosine and cytidine (with the abbreviation, “C”), uracil and uridine (with the abbreviation, “U”), thymine and thymidine (with the abbreviation, “T”), inosine and hypo-xanthine (with the abbreviation, “I”), are used interchangeably to refer to the corresponding nucleobase, nucleoside or nucleotide. Sometimes the terms nucleobase, nucleoside and nucleotide are used interchangeably, unless the context clearly requires differently.

The term “introducing” or “introduction” used herein means delivering one or more polynucleotides, such as dRNAs or one or more constructs including vectors as described herein, one or more transcripts thereof, to a host cell. The invention serves as a basic platform for enabling targeted editing of RNA, for example, pre-messenger RNA, a messenger RNA, a ribosomal RNA, a transfer RNA, a long non-coding RNA and a small RNA (such as miRNA). The methods of the present application can employ many delivery systems, including but not limited to, viral, liposome, electroporation, microinjection and conjugation, to achieve the introduction of the dRNA or construct as described herein into a host cell. Conventional viral and non-viral based gene transfer methods can be used to introduce nucleic acids into mammalian cells or target tissues. Such methods can be used to administer nucleic acids encoding dRNA of the present application to cells in culture, or in a host organism. Non-viral vector delivery systems include DNA plasmids, RNA (e.g. a transcript of a construct described herein), naked nucleic acid, and nucleic acid complexed with a delivery vehicle, such as a liposome. Viral vector delivery systems include DNA and RNA viruses, which have either episomal or integrated genomes for delivery to the host cell.

In the context of the present application, “target RNA” refers to an RNA sequence to which a deaminase-recruiting RNA sequence is designed to have perfect complementarity or substantial complementarity, and hybridization between the target sequence and the dRNA forms a double stranded RNA (dsRNA) region containing a target adenosine, which recruits an adenosine deaminase acting on RNA (ADAR) that deaminates the target adenosine. In some embodiments, the ADAR is naturally present in a host cell, such as a eukaryotic cell (such as a mammalian cell, e.g., a human cell). In some embodiments, the ADAR is introduced into the host cell.

As used herein, “complementarity” refers to the ability of a nucleic acid to form hydrogen bond(s) with another nucleic acid by traditional Watson-Crick base-pairing. A percent complementarity indicates the percentage of residues in a nucleic acid molecule which can form hydrogen bonds (i.e., Watson-Crick base pairing) with a second nucleic acid (e.g., about 5, 6, 7, 8, 9, 10 out of 10, being about 50%, 60%, 70%, 80%, 90%, and 100% complementary respectively). “Perfectly complementary” means that all the contiguous residues of a nucleic acid sequence form hydrogen bonds with the same number of contiguous residues in a second nucleic acid sequence. “Substantially complementary” as used herein refers to a degree of complementarity that is at least about any one of 70%, 75%, 80%, 85%, 90%, 95%, 97%, 98%, 99%, or 100% over a region of about 40, 50, 60, 70, 80, 100, 150, 200, 250 or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology—Hybridization With Nucleic Acid Probes Part I, Second Chapter “Overview of principles of hybridization and the strategy of nucleic acid probe assay,” Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. A sequence capable of hybridizing with a given sequence is referred to as the “complement” of the given sequence.

As used herein, the terms “including,” “containing,” and “comprising” are used in their open, non-limiting sense. It is also understood that aspects and embodiments of the present application described herein may include “consisting” and/or “consisting essentially of” aspects and embodiments.

It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

As used herein, a “carrier” includes pharmaceutically acceptable carriers, excipients, or stabilizers that are nontoxic to the cell or mammal being exposed thereto at the dosages and concentrations employed. Often the physiologically acceptable carrier is an aqueous pH buffered solution. Non-limiting examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

As used herein, the term “effective amount” or “therapeutically effective amount” of a substance is at least the minimum concentration required to effect a measurable improvement or prevention of a particular disorder. An effective amount herein may vary according to factors such as the disease state, age, sex, and weight of the patient, and the ability of the substance to elicit a desired response in the individual. An effective amount is also one in which any toxic or detrimental effects of the treatment are outweighed by the therapeutically beneficial effects. In reference to cancer, an effective amount comprises an amount sufficient to cause a tumor to shrink and/or to decrease the growth rate of the tumor (such as to suppress tumor growth) or to prevent or delay other unwanted cell proliferation in cancer. In some embodiments, an effective amount is an amount sufficient to delay development of cancer. In some embodiments, an effective amount is an amount sufficient to prevent or delay recurrence. In some embodiments, an effective amount is an amount sufficient to reduce recurrence rate in the individual. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) reduce recurrence rate of tumor, and/or (viii) relieve to some extent one or more of the symptoms associated with the cancer. An effective amount can be administered in one or more administrations. For purposes of this disclosure, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

A “host cell” as described herein refers to any cell type that can be used as a host cell provided it can be modified as described herein. For example, the host cell may be a host cell with endogenously expressed adenosine deaminase acting on RNA (ADAR), or may be a host cell into which an adenosine deaminase acting on RNA (ADAR) is introduced by a known method in the art. For example, the host cell may be a prokaryotic cell, a eukaryotic cell or a plant cell. In some embodiments, the host cell is derived from a pre-established cell line, such as mammalian cell lines including human cell lines or non-human cell lines. In some embodiments, the host cell is derived from an individual, such as a human individual.

A “recombinant AAV vector (rAAV vector)” refers to a polynucleotide vector comprising one or more heterologous sequences (i.e., nucleic acid sequence not of AAV origin) that are flanked by at least one, and in embodiments two, AAV inverted terminal repeat sequences (ITRs). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. An rAAV vector can be in any of a number of forms, including, but not limited to, plasmids, linear artificial chromosomes, complexed with lipids, encapsulated within liposomes, and encapsidated in a viral particle, particularly an AAV particle. A rAAV vector can be packaged into an AAV virus capsid to generate a “recombinant adeno-associated viral particle (rAAV particle)”.

An “AAV inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C′ and D regions), allowing intrastrand base-pairing to occur within this portion of the ITR.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments, etc.

A “subject,” “patient” or “individual” includes a mammal, such as a human or other animal, and typically is human. In some embodiments, the subject, e.g., patient, to whom the therapeutic agents and compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent, a dog, a cat, a farm animal, such as a cow or a horse, etc.

As used herein, the term “treatment” refers to clinical intervention designed to have beneficial and desired effects to the natural course of the individual or cell being treated during the course of clinical pathology. For the purpose of this disclosure, desirable effects of treatment include, without limitation, decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, increasing cancer cell-killing, decreasing symptoms resulting from the disease, preventing spread of diseases, preventing recurrence of disease, increasing the quality of life of those suffering from the disease, decreasing the dose of other medications required to treat the disease, delaying the progression of the disease, and/or prolonging survival of individuals.

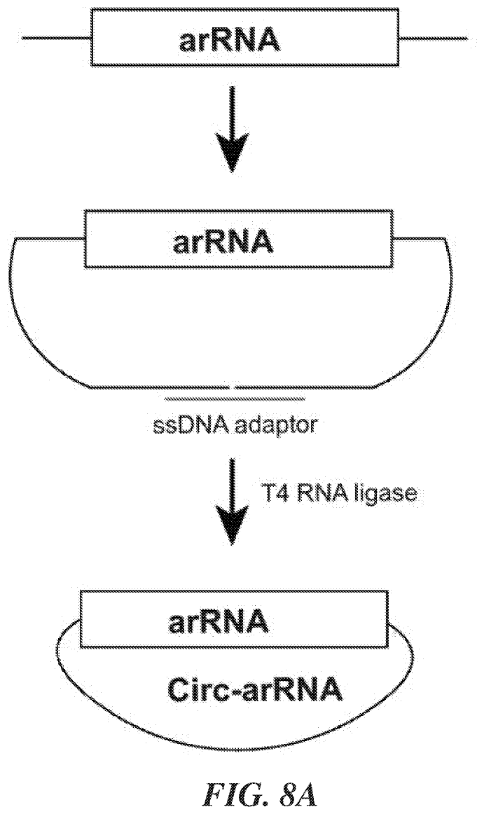

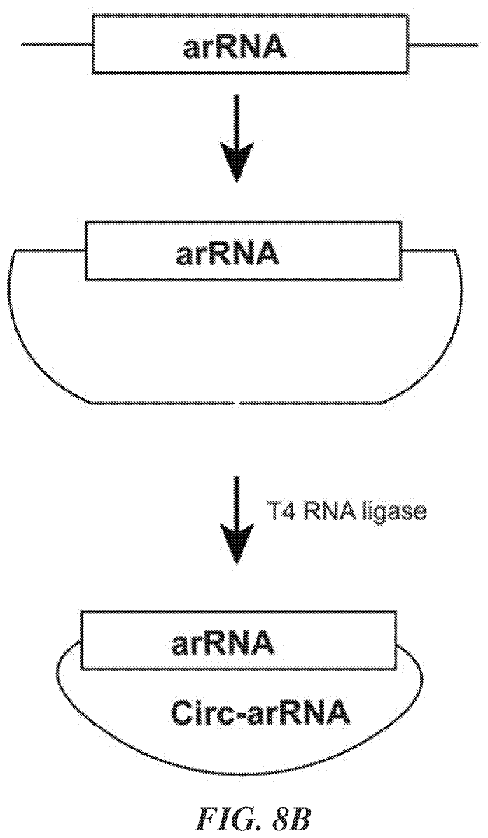

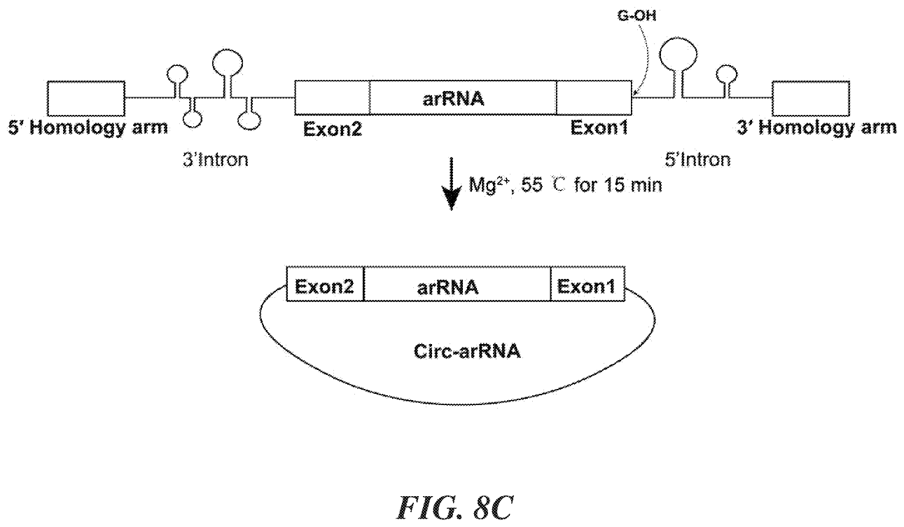

The terms “Group I intron” and “Group I catalytic intron” are used interchangeably to refer to a self-splicing ribozyme that can catalyze its own excision from an RNA precursor. Group I introns comprise two fragments, the 5′ catalytic Group I intron fragment and the 3′ catalytic Group I intron fragment, which retain their folding and catalytic function (i.e., self-splicing activity). In its native environment, the 5′ catalytic Group I intron fragment is flanked at its 5′ end by a 5′ exon, which comprises a 5′ exon sequence that is recognized by the 5′ catalytic Group I intron fragment; and the 3′ catalytic Group I intron fragment is flanked at its 3′ end by a 3′ exon, which comprises a 3′ exon sequence that is recognized by the 3′ catalytic Group I intron fragment. The terms “5′ exon sequence” and “3′ exon sequence” used herein are labeled according to the order of the exons with respect to the Group I intron in its natural environment, e.g., as shown in FIG. 8C.

### II. METHODS OF RNA EDITING

Provided herein are methods for editing a target RNA in a host cell, comprising introducing a deaminase-recruiting RNA (dRNA) or a construct comprising a nucleic acid encoding the dRNA into the host cell (e.g., eukaryotic cell), wherein: (1) the dRNA comprises a targeting RNA sequence that is at least partially complementary to the target RNA and (2) the dRNA is capable of recruiting an adenosine deaminase acting on RNA (ADAR).

Circular dRNAs

In one aspect, provided herein are methods for editing a target RNA in a host cell, comprising introducing a deaminase-recruiting RNA (dRNA) or a construct comprising a nucleic acid encoding the dRNA into the host cell (e.g., eukaryotic cell), wherein: (1) the dRNA comprises a targeting RNA sequence that is at least partially complementary to the target RNA, (2) the dRNA is capable of recruiting an adenosine deaminase acting on RNA (ADAR), and (3) the dRNA is a circular RNA or capable of forming a circular RNA in the host cell.

In some embodiments, provided herein are methods for editing a target RNA in a host cell, comprising introducing a deaminase-recruiting RNA (dRNA) or a construct comprising a nucleic acid encoding the dRNA into the host cell, wherein: (1) the dRNA comprises a targeting RNA sequence that is at least partially complementary to the target RNA, (2) the dRNA is capable of recruiting an endogenously expressed ADAR of the host cell to deaminate a target adenosine residue in the target RNA, and (3) the dRNA is a circular RNA or capable of forming a circular RNA in the host cell. In some embodiments, the method does not comprise introducing any protein or construct comprising a nucleic acid encoding a protein (e.g., Cas, ADAR or a fusion protein of ADAR and Cas) to the host cell.

In some embodiments, provided herein are methods for editing a target RNA in a host cell, comprising introducing a deaminase-recruiting RNA (dRNA) or a construct comprising a nucleic acid encoding the dRNA into the host cell, wherein: (1) the dRNA comprises a targeting RNA sequence that is at least partially complementary to the target RNA, (2) the dRNA is capable of recruiting an endogenously expressed ADAR of the host cell to deaminate a target adenosine residue in the target RNA, and (3) the dRNA is a circular RNA. In some embodiments, the method does not comprise introducing any protein or construct comprising a nucleic acid encoding a protein (e.g., Cas, ADAR or a fusion protein of ADAR and Cas) to the host cell.

In some embodiments, provided herein are methods for editing a target RNA in a host cell, comprising introducing a deaminase-recruiting RNA (dRNA) or a construct comprising a nucleic acid encoding the dRNA into the host cell, wherein: (1) the dRNA comprises a targeting RNA sequence that is at least partially complementary to the target RNA, (2) the dRNA is capable of recruiting an endogenously expressed ADAR of the host cell to deaminate a target adenosine residue in the target RNA, and (3) the dRNA is a linear RNA capable of forming a circular RNA in the host cell. In some embodiments, the method does not comprise introducing any protein or construct comprising a nucleic acid encoding a protein (e.g., Cas, ADAR or a fusion protein of ADAR and Cas) to the host cell.

In some embodiments, provided herein are methods for editing a target RNA in a host cell, comprising introducing a deaminase-recruiting RNA (dRNA) or a construct comprising a nucleic acid encoding the dRNA into the host cell and an ADAR or a construct comprising a nucleic acid encoding the ADAR into the host cell, wherein: (1) the dRNA comprises a targeting RNA sequence that is at least partially complementary to the target RNA, (2) the dRNA is capable of recruiting the ADAR to deaminate a target adenosine (“A”) residue in the target RNA, and (3) the dRNA is a circular RNA or capable of forming a circular RNA in the host cell. In some embodiments, the ADAR is an endogenously encoded ADAR of the host cell, wherein introduction of the ADAR comprises over-expressing the ADAR in the host cell. In some embodiments, the ADAR is exogenous to the host cell. In some embodiments, the construct comprising a nucleic acid encoding the ADAR is a vector, such as a plasmid, or a viral vector (e.g., an AAV or a lentiviral vector).

In one aspect, the present application provides a method for editing a plurality of target RNAs (e.g., at least about 2, 3, 4, 5, 10, 20, 50, 100, 1000 or more) in host cells by introducing a plurality of the dRNAs, or one or more constructs encoding the dRNAs, into the host cells.

In one aspect, the dRNA is a linear RNA that is capable of forming a circular RNA in a host cell. In some embodiments, the circulation is performed using the Tornado expression system (“Twister-optimized RNA for durable overexpression”) as described in Litke, J. L. & Jaffrey, S. R. Highly efficient expression of circular RNA aptamers in cells using autocatalytic transcripts. Nat Biotechnol 37, 667-675 (2019), which is hereby incorporated herein by reference in its entirety. Briefly, Tornado-expressed transcripts contain an RNA of interest flanked by Twister ribozymes. A twister ribozyme is any catalytic RNA sequences that are capable of self-cleavage. The ribozymes rapidly undergo autocatalytic cleavage, leaving termini that are ligated by an RNA ligase.

In some embodiments, the dRNA is introduced by a construct comprising a nucleic acid encoding the dRNA. In some embodiments, the construct further comprises a 3′ twister ribozyme sequence linked to the 3′ end of the nucleic acid encoding the dRNA. In some embodiments, the construct further comprises a 5′ twister ribozyme sequence linked to the 5′ end of the nucleic acid encoding the dRNA. In some embodiments, the construct further comprises a 3′ twister ribozyme sequence linked to the 3′ end of the nucleic acid encoding the dRNA and a 5′ twister ribozyme sequence linked to the 5′ end of the nucleic acid encoding the dRNA. In some embodiments, the 3′ twister sequence is twister P3 U2A and the 5′ twister sequence is twister P1. In some embodiments, wherein the 5′ twister sequence is twister P3 U2A and the 3′ twister sequence is twister P1. In some embodiments, the dRNA undergoes autocatalytic cleavage. In some embodiments, the catalyzed dRNA product comprises a 5′-hydroxyl group and a 2′,3′-cyclic phosphate at the 3′ terminus. In some embodiments, the catalyzed dRNA product is ligated by ubiquitous endogenous RNA ligase (e.g., RNA ligase RtcB). In some embodiments, the construct is a plasmid or a viral vector.

In some embodiments, the dRNA transcript is also flanked by a 5′ and/or 3′ ligation sequences, which are then flanked by the 5′-Twister ribozyme and/or 3′-Twister ribozymes, respectively. In some embodiments, the dRNA comprises a 3′ ligation sequence. In some embodiments, the dRNA comprises a 5′ ligation sequence. In some embodiments, the dRNA further comprises a 3′ ligation sequence and a 5′ ligation sequence. In some embodiments, the 3′ ligation sequence and the 5′ ligation sequence are at least partially complementary to each other. In some embodiments, the 3′ ligation sequence and the 5′ ligation sequence are at least about 50%, at least about 55%, at least about 60%, at least about 65%, at least about 70%, at least about 75%, at least about 80%, at least about 85%, at least about 90%, at least about 95%, at least about 96%, at least about 97%, at least about 98% or at least about 99% complementary to each other. In some embodiments, the 3′ ligation sequence and the 5′ ligation sequence are fully complementary to each other.

In some embodiments, the 3′ ligation sequence and the 5′ ligation sequence are independently at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides or at least about 100 nucleotides in length. In some embodiments, the 3′ ligation sequence and the 5′ ligation sequence are independently about 20-30 nucleotides, about 30-40 nucleotides, about 40-50 nucleotides, about 50-60 nucleotides, about 60-70 nucleotides, about 70-80 nucleotides, about 80-90 nucleotides, about 90-100 nucleotides, about 100-125 nucleotides, about 125-150 nucleotides, about 20-50 nucleotides, about 50-100 nucleotides or about 100-150 nucleotides in length.

In some embodiments, the dRNA is circularized by an RNA ligase. Non-limiting examples of RNA ligase include: RtcB, T4 RNA Ligase 1, T4 RNA Ligase 2, Rnl3 and Trl1. In some embodiments, the RNA ligase is expressed endogenously in the host cell. In some embodiments, the RNA ligase is RNA ligase RtcB. In some embodiments, the method further comprises introducing an RNA ligase (e.g., RtcB) into the host cell.

In some embodiments, the dRNA is circularized before being introduced to the host cell. In some embodiments, the dRNA is a circular RNA formed from a linear RNA in vitro. In some embodiments, the dRNA is chemically synthesized. In some embodiments, the dRNA is circularized through in vitro enzymatic ligation (e.g., using RNA or DNA ligase, or via splicing of intron fragments) or chemical ligation (e.g., using cyanogen bromide or a similar condensing agent).

dRNA Having One or Two snoRNA Ends

In another aspect, provided herein are methods for editing a target RNA in a host cell (e.g., eukaryotic cell), comprising introducing a deaminase-recruiting RNA (dRNA) or a construct comprising a nucleic acid encoding the dRNA into the host cell, wherein the dRNA comprises: (1) a targeting RNA sequence that is at least partially complementary to the target RNA and (2) a small nucleolar RNA (snoRNA) sequence linked to the 3′ and/or 5′ ends of the targeting RNA sequence; and wherein the dRNA is capable of recruiting an adenosine deaminase acting on RNA (ADAR).

In some embodiments, provided herein are methods for editing a target RNA in a host cell (e.g., eukaryotic cell), comprising introducing a deaminase-recruiting RNA (dRNA) or a construct comprising a nucleic acid encoding the dRNA into the host cell, wherein the dRNA comprises: (1) a targeting RNA sequence that is at least partially complementary to the target RNA and (2) a small nucleolar RNA (snoRNA) sequence linked to the 3′ and/or 5′ ends of the targeting RNA sequence; and wherein the dRNA is capable of recruiting an endogenously expressed adenosine deaminase acting on RNA (ADAR) of the host cell to deaminate a target adenosine residue in the target RNA. In some embodiments, the method does not comprise introducing any protein or construct comprising a nucleic acid encoding a protein (e.g., Cas, ADAR or a fusion protein of ADAR and Cas) to the host cell.

In some embodiments, provided herein are methods for editing a target RNA in a host cell (e.g., eukaryotic cell), comprising introducing (a) a deaminase-recruiting RNA (dRNA) or a construct comprising a nucleic acid encoding the dRNA and (b) an ADAR or a construct comprising a nucleic acid encoding the ADAR into the host cell, wherein the dRNA comprises: (1) a targeting RNA sequence that is at least partially complementary to the target RNA and (2) a small nucleolar RNA (snoRNA) sequence linked to the 3′ and/or 5′ ends of the targeting RNA sequence; and wherein the dRNA is capable of recruiting the ADAR to deaminate a target adenosine residue in the target RNA. In some embodiments, the ADAR is an endogenously encoded ADAR of the host cell, wherein introduction of the ADAR comprises over-expressing the ADAR in the host cell. In some embodiments, the ADAR is exogenous to the host cell. In some embodiments, the construct comprising a nucleic acid encoding the ADAR is a vector, such as a plasmid, or a viral vector (e.g., an AAV or a lentiviral vector).

In one aspect, the present application provides a method for editing a plurality of target RNAs (e.g., at least about 2, 3, 4, 5, 10, 20, 50, 100, 1000 or more) in host cells by introducing a plurality of the dRNAs, or one or more constructs encoding the dRNAs, into the host cells.

Small nucleolar RNAs (snoRNAs) are small non-coding RNA molecules that are known to guide chemical modifications of other RNAs such as ribosomal RNAs, transfer RNAs, and small nuclear RNAs. There are two major groups of snoRNAs according to their specific secondary structure features: box C/D and box H/ACA. Both structural features of snoRNAs enable them binding to corresponding RNA binding proteins (RBPs) along with accessory proteins, forming functional small nucleolar ribonucleoprotein (snoRNP) complexes. Box C/D snoRNAs are believed to be associated with methylation, while H/ACA box snoRNAs are believed to be associated with pseudouridylation. Other families of snoRNAs include, for example, composite H/ACA and C/D box snoRNA and orphan snoRNAs. The snoRNA sequence described herein can comprise a naturally-occurring snoRNA, a portion thereof, or a variant thereof.

In some embodiments, the dRNA comprises a snoRNA sequence linked to the 5′ end of the targeting RNA sequence (“5′ snoRNA sequence”). In some embodiments, the dRNA comprises a snoRNA sequence linked to the 3′ end of the targeting RNA sequence (“3′ snoRNA sequence”). In some embodiments, the dRNA comprises a snoRNA sequence linked to the 5′ end of the targeting RNA sequence (“5′ snoRNA sequence”) and a snoRNA sequence linked to the 3′ end of the targeting RNA sequence (“3′ snoRNA sequence”). In some embodiments, the snoRNA sequence is at least about 50 nucleotides, at least about 60 nucleotides, at least about 70 nucleotides, at least about 80 nucleotides, at least about 90 nucleotides, at least about 100 nucleotides, at least about 110 nucleotides, at least about 120 nucleotides, at least about 130 nucleotides, at least about 140 nucleotides, at least about 150 nucleotides, at least about 160 nucleotides, at least about 170 nucleotides, at least about 180 nucleotides, at least about 190 nucleotides or at least about 200 nucleotides in length. In some embodiments, the snoRNA sequence is about 50-75 nucleotides, about 75-100 nucleotides, about 100-125 nucleotides, about 125-150 nucleotides, about 150-175 nucleotides, about 175-200 nucleotides, about 50-100 nucleotides, about 100-150 nucleotides, about 150-200 nucleotides, about 125-175 nucleotides, or about 100-200 nucleotides in length.

In some embodiments, the 3′ snoRNA sequence comprises the nucleic acid sequence of SEQ ID NO: 1 (5′-AAGATTGTGTGTGGATCGATGATGACTTCCATATATACATTCCTTGGAAAGCTGAAC AAAATGAGTGAAAACTCTATACCGTCATTCTCGTCGAACTGAGGTCCAGCACATTAC TCCAACAG-3′). In some embodiments, the 5′ snoRNA sequence comprises the nucleic acid sequence of SEQ ID NO: 2 (5′-GAGTGAGATCTTGGACCAATGATGACTTCCATACATGCATTCCTTGGAAAGCTGAAC AAAATGAGTGGGAACTCTGTACTATCATCTTAGTTGAACTGAGGTCCACCGGGGGCT AA-3′). In some embodiments, the snoRNA sequence is a C/D Box snoRNA sequence. In some embodiments, the snoRNA sequence is an H/ACA Box snoRNA sequence. In some embodiments, the snoRNA sequence is a composite C/D Box and H/ACA Box snoRNA sequence. In some embodiments, the snoRNA sequence is an orphan snoRNA sequence.

Circular dRNA Formed from a Linear RNA In Vitro

In some embodiments, the circular RNA is formed by in vitro splicing of intron fragments, such as Group I catalytic intron fragments. In some embodiments, the dRNA is a circular RNA comprising a 3′ exon sequence recognizable by a 3′ catalytic Group I intron fragment flanking the 5′ end of the nucleic acid sequence encoding the dRNA, and a 5′ exon sequence recognizable by a 5′ catalytic Group I intron fragment flanking the 3′ end of the nucleic acid sequence encoding the dRNA. In some embodiments, the 3′ exon sequence recognizable by a 3′ catalytic Group I intron fragment comprises the nucleic acid sequence of SEQ ID NO: 54. In some embodiments, the 5′ exon sequence recognizable by a 5′ catalytic Group I intron fragment comprises the nucleic acid sequence of SEQ ID NO: 55. In some embodiments, the 3′ exon sequence comprises the nucleic acid sequence of SEQ ID NO: 54, and the 5′ exon sequence comprises the nucleic acid sequence of SEQ ID NO: 55.

(3′ exon sequence recognizable
by a 3′ catalytic Group I intron
fragment)
SEQ ID NO: 54
AAAAUCCGUUGACCUUAAACGGUCGUGUGGGUUCA
AGUCCCUCCACCCCCAC
(5′ exon sequence recognizable by a
5′ catalytic Group I intron fragment)
SEQ ID NO: 55
GAGACGCUACGGACUU

In some embodiments, the Group I catalytic intron of the T4 phage Td gene is bisected in such a way to preserve structural elements critical for ribozyme folding. Exon fragment 2 is then ligated upstream of exon fragment 1, and a nucleic acid sequence encoding the dRNA is inserted between the exon-exon junction.

In some embodiments, the circular RNA is formed by in vitro ligation of a linear RNA comprising a 5′ ligation sequence at the 5′ end of the linear RNA, and a 3′ ligation sequence at the 3′ end of the linear RNA, wherein the 5′ ligation sequence and the 3′ ligation sequence are ligated to each other via a ligase (e.g., T4 RNA ligase).

The present application further provides nucleic acid constructs (e.g., linear RNA and vectors, etc.) for preparation of the circular dRNAs described herein, and methods for preparing the circular dRNAs, for example, by chemical ligation, enzymatic ligation, or ribozyme autocatalysis of linear RNAs. The linear RNAs are also referred herein as “linear RNA precursors.”

In some embodiments, the methods described herein comprise any one or more steps of methods of forming the circular dRNA in vitro, including, for example, steps of ligation or splicing in vitro, transcription of the linear RNA precursor in vitro, and isolation and purification of the circular dRNA. These steps and components are described in further detail in sections a)-e) below.

### a) Linear RNA Circularized by In Vitro Splicing

In some embodiments, the present application provides a linear RNA capable of forming the circular dRNA of any one of the embodiments described herein. In some embodiments, the linear RNA can circularized by chemical circularization methods using cyanogen bromide or a similar condensing agent. In some embodiments, the linear RNA can be circularized by autocatalysis of a Group I intron comprising a 5′ catalytic Group I intron fragment and a 3′ catalytic Group I intron fragment. In some embodiments, the linear RNA can be circularized by a ligase. In some embodiments, the linear RNA can be circularized by a T4 RNA ligase. In some embodiments, the linear RNA can be circularized by a DNA ligase. Suitable ligases include, but are not limited to a T4 DNA ligase (T4 Dnl), a T4 RNA ligase 1 (T4 Rnl1) and a T4 RNA ligase 2 (T4 Rnl2).

In some embodiments, the present application provides a linear RNA capable of forming the circular dRNA of any one of the embodiments described herein, wherein the linear RNA can be circularized by autocatalysis of a Group I intron. In some embodiments, the Group I intron comprises a 5′ catalytic Group I intron fragment and a 3′ catalytic Group I intron fragment. In some embodiments, the linear RNA comprises a 3′ catalytic Group I intron fragment (such as the sequence set forth in SEQ ID NO: 56) flanking the 5′ end of a 3′ exon sequence recognizable by the 3′ catalytic Group I intron fragment (such as the sequence set forth in SEQ ID NO: 54), and the 5′ catalytic Group I intron fragment (such as the sequence set forth in SEQ ID NO: 57) flanking the 3′ end of a 5′ exon sequence recognizable by the 5′ catalytic Group I intron fragment (such as the sequence set forth in SEQ ID NO: 55).

(3′ catalytic Group I intron fragment)
SEQ ID NO: 56
AACAAUAGAUGACUUACAACUAAUCGGAAGGUGCAGAGACUCGAC
GGGAGCUACCCUAACGUCAAGACGAGGGUAAAGAGAGAGUCCAAU
UCUCAAAGCCAAUAGGCAGUAGCGAAAGCUGCAAGAGAAUG
(5′ catalytic Group I intron fragment)
SEQ ID NO: 57
AAAUAAUUGAGCCUUAAAGAAGAAAUUCUUUAAGUGGAUGCUCUC
AAACUCAGGGAAACCUAAAUCUAGUUAUAGACAAGGCAAUCCUGA
GCCAAGCCGAAGUAGUAAUUAGUAAG

In some embodiments, the linear RNA further comprises a 5′ homology sequence flanking the 5′ end of the 3′ catalytic Group I intron fragment, and a 3′ homology sequence flanking the 3′ end of the 5′ catalytic Group I intron fragment. In some embodiments, the linear RNA comprises, from 5′ to 3′ end, a 5′homology arm-3′catalytic Group I Intron fragment-3′ exon sequence-dRNA-5′ exon sequence-5′ catalytic Group I Intron fragment-3′homology arm sequence. In some embodiments, the homology sequence can be between 1 and 100, between 5 and 80, between 5 and 60, between 10 and 50, or between 12 and 50 nucleotides in length. In some embodiments, the homology sequence is about 20-30 nucleotides in length. In some embodiments, the 5′ homology sequence comprises the nucleic acid sequence of SEQ ID NO: 58, and the 3′ homology sequence comprises the nucleic acid sequence of SEQ ID NO: 59. In some embodiments, the homology arms increase the efficiency of RNA circularization by about 0 to 20%, more than 20%, more than 30%, more than 40%, or more than 50%.

(Exemplary 5′ homology sequence)
SEQ ID NO: 58
GGGAGACCCUCGACCGUCGAUUGUCCACUGGUC
(Exemplary 3′ homology sequence)
SEQ ID NO: 59
ACCAGUGGACAAUCGACGGAUAACAGCAUAUCUAG

In some embodiments, there is provided a nucleic acid construct comprising a nucleic acid sequence encoding the linear RNA. In some embodiments, a T7 promoter is operably linked to the nucleic acid sequence encoding the linear RNA. In some embodiments, the T7 promoter comprises the sequence set forth in SEQ ID NO: 60. In some embodiments, the T7 promoter is capable of driving in vitro transcription.

(T7 Promoter)
SEQ ID NO: 60
UAAUACGACUCACUAUAGG

In some embodiments, the method described herein comprises circularizing a linear RNA by ribozyme autocatalysis or splicing of catalytic introns in vitro. In some embodiments, method comprises (a) subjecting the linear RNA described herein to a condition that activates autocatalysis of the Group I intron (or 5′ and 3′ catalytic Group I intron fragments thereof) to provide a circularized RNA product; and (b) isolating the circularized RNA product, thereby providing the circular dRNA.

In some embodiments, the method comprises a step of obtaining the linear RNA by first cloning the sequence encoding the linearized RNAs into a plasmid vector, and then linearizing the recombinant plasmids. In some embodiments, the recombinant plasmids are linearized by restriction enzyme digestion. In some embodiments, the recombinant plasmids are linearized by PCR amplification. In some embodiments, the method further comprises performing in vitro transcription with the linearized plasmid template. In some embodiments, the in vitro transcription is driven by a T7 promoter. In some embodiments, the method further comprises purifying the linear RNA transcripts. In some embodiments, the linear RNAs are purified by gel purification.

In some embodiments, the present application provides a method of cyclizing a linear RNA (e.g., purified linear RNA) by ribozyme autocatalysis of the Group I intron. During splicing, the 3′ hydroxyl group of a guanosine nucleotide engages in a transesterification reaction at the 5′ splice site. The 5′ intron half is excised and the freed hydroxyl group at the end of the intermediate engages in a second transesterification at the 3′ splice site, resulting in circularization of the intervening region and excision of the 3′ intron. In some embodiments, the condition that activates autocatalysis of the Group I intron or 5′ and 3′ catalytic Group I intron fragments is the addition of GTPs and Mg²⁺. In some embodiments, there is provided a step of cyclizing the linear RNAs by adding GTPs and Mg²⁺ at 55° C. for 15 min. In some embodiments, the method further comprises treating the reaction mixture with RNase R to digest the linear RNA transcripts. In some embodiments, the method further comprises isolating the circular dRNA.

In some embodiments, the circularization has an efficiency of at least 2%, at least 5%, at least 10%, at least 15%, at least 20%, at least 25%, at least 30%, at least 32%, at least 34%, at least 36%, at least 38%, at least 40%, at least 42%, at least 44%, at least 46%, at least 48%, or at least 50%. In some embodiments, the circularization has an efficiency of about 40% to about 50% or more than 50%.

### b) Linear RNA Circularized by Ligation

In some embodiments, the circular RNA is formed by in vitro ligation of a linear RNA using a ligase such as a RNA ligase. In some embodiments, the linear RNA can be circularized by a T4 RNA ligase. In some embodiments, the linear RNA comprises a 5′ ligation sequence at the 5′ end of the nucleic acid sequence encoding the circular dRNA, and a 3′ ligation sequence at the 3′ end of the nucleic acid sequence encoding the circular dRNA, wherein the 5′ ligation sequence and the 3′ ligation sequence can be ligated to each other via the RNA ligase. In non-limiting examples, the linear RNA can be circularized by a ligase such as a T4 DNA ligase (T4 Dnl), T4 RNA ligase 1 (T4 Rnl1), and T4 RNA ligase 2 (T4 Rnl2). The linear RNA may be circularized with or without the presence of a single stranded nucleic acid adaptor, e.g., a splint DNA.

In some embodiments, the method described herein comprises circularizing a linear RNA in vitro, comprising: (a) contacting any one of the linear RNAs comprising a 5′ ligation sequence at the 5′ end of the nucleic acid sequence encoding the circular dRNA, and a 3′ ligation sequence at the 3′ end of the nucleic acid sequence encoding the circular dRNA described above with a single-stranded adaptor nucleic acid comprising from the 5′ end to the 3′ end: a first sequence complementary to the 3′ ligation sequence and a second sequence complementary to the 5′ ligation sequence, and wherein the 5′ ligation sequence and the 3′ ligation sequence hybridize to the single-stranded adaptor nucleic acid to provide a duplex nucleic acid intermediate comprising a single strand break between the 3′ end of the 5′ ligation sequence and the 5′ end of the 3′ ligation sequence; (b) contacting the intermediate with an RNA ligase under a condition that allows ligation of the 5′ ligation sequence to the 3′ ligation sequence to provide a circularized RNA product; and (c) isolating the circularized RNA product, thereby providing the circular dRNA.

In some embodiments, the method described herein comprises circularizing a linear RNA in vitro, comprising: (a) contacting any one of the linear RNAs comprising a 5′ ligation sequence at the 5′ end of the nucleic acid sequence encoding the circular dRNA, and a 3′ ligation sequence at the 3′ end of the nucleic acid sequence encoding the circular dRNA described above with an RNA ligase under a condition that allows ligation of the 5′ ligation sequence to the 3′ ligation sequence to provide a circularized RNA product; and (b) isolating the circularized RNA product, thereby providing the circular dRNA.

In some embodiments, the method further comprises treating the reaction mixture with RNase R to digest the linear RNA transcripts. In some embodiments, the method further comprises isolating the circular dRNA.

In some embodiments, a DNA or RNA ligase may be used to enzymatically link a 5′-phosphorylated nucleic acid molecule (e.g., a linear RNA) to the 3′-hydroxyl group of a nucleic acid (e.g., a linear nucleic acid) forming a new phosphodiester linkage. In an example reaction, a linear circular RNA is incubated at 37° C. for 1 hour with 1-10 units of T4 RNA ligase (New England Biolabs, Ipswich, Mass.) according to the manufacturer's protocol. The ligation reaction may occur in the presence of a linear nucleic acid capable of base pairing with both the 5′- and 3′-region in juxtaposition to assist the enzymatic ligation reaction. In some embodiments, the ligation is splint ligation. For example, a splint ligase, like SPLINTR® ligase, can be used for splint ligation. For splint ligation, a single stranded polynucleotide (splint), like a single stranded RNA, can be designed to hybridize with both termini of a linear polyribonucleotide, so that the two termini can be juxtaposed upon hybridization with the single-stranded splint. Splint ligase can thus catalyze the ligation of the juxtaposed two termini of the linear polyribonucleotide, generating a circular polyribonucleotide.

In some embodiments, a DNA or RNA ligase may be used in the synthesis of the circular RNA. As a non-limiting example, the ligase may be a circ ligase or circular ligase.

### c) Linear RNA Circularized by Chemical Ligation

In some embodiments, the method described herein comprises circularizing a linear RNA in vitro, comprising: (a) chemically ligating the 5′ end and the 3′ end of a linear RNA comprising a nucleic acid sequence encoding the circular dRNA; and (b) isolating the circularized RNA product, thereby providing the circular dRNA.

In some embodiments, the step of circularizing the linear RNA comprises chemical circularization methods using cyanogen bromide or a similar condensing agent.

In some embodiments, a linear RNA precursor can be circularized by chemical methods. In some embodiments, the 5′-end and the 3′-end of the nucleic acid (e.g., a linear circular polyribonucleotide) includes chemically reactive groups that, when close together, may form a new covalent linkage between the 5′-end and the 3′-end of the molecule. The 5′-end may contain an NHS ester reactive group and the 3′-end may contain a 3′-amino terminated nucleotide such that in an organic solvent the 3′-amino-terminated nucleotide on the 3′-end of a linear RNA molecule will undergo a nucleophilic attack on the 5′-NHS-ester moiety forming a new 5′-/3′-amide bond.

In some embodiments, the circularization efficiency of the circularization methods provided herein is at least about 10%, at least about 15%, at least about 20%, at least about 25%, at least about 30%, at least about 35%, at least about 40%, at least about 45%, at least about 50%, at least about 60, at least about 70%, at least about 80%, at least about 90%, at least about 95%, or 100%. In some embodiments, the circularization efficiency of the circularization methods provided herein is at least about 40%.

### d) Plasmids

In some embodiments, the present application provides plasmids comprising the nucleotide sequences described herein. In some embodiments, the plasmids are obtained by cloning the sequence encoding the linear RNAs into a plasmid vector. Plasmids can be generated by techniques known in the art, such as Gibson cloning or cloning using restriction enzymes. In some embodiments, the plasmid vector includes an antibiotic expression cassette allowing antibiotic selection of bacteria expressing the plasmid. In some embodiments, the plasmids provided can be purified from bacteria and used for production of the circular RNAs. Any plasmid vector suitable for in vitro transcription of the linear RNA may be used.

In some embodiments, the plasmids are linearized prior to in vitro transcription of the linear RNA. In some embodiments, the recombinant plasmids are linearized by restriction enzyme digestion. In some embodiments, the recombinant plasmids are linearized by PCR amplification. In some embodiments, the method further comprises performing in vitro transcription with the linearized plasmid template. In some embodiments, the in vitro transcription is driven by a T7 promoter.

e) Purification of Circular dRNA

In some embodiments, the method described herein further comprises a step of isolating and/or purifying the circularized RNA product. In some embodiments, the method comprises gel-purifying the circular dRNA. In some embodiments, agarose gel electrophoresis allows for simple and effective separation of circular splicing products from linear precursor molecules, nicked circles, splicing intermediates, and excised introns. In some embodiments, the method comprises purifying the circular dRNA by chromatography, such as HPLC. In some embodiments, the purified circular dRNA can be stored at −80° C.

Constructs with Pol H Promoter

In another aspect, provided herein are methods for editing a target RNA in a host cell, comprising introducing a construct comprising a nucleic acid encoding a deaminase-recruiting RNA (dRNA) into the host cell, wherein: (1) the dRNA comprises a targeting RNA sequence that is at least partially complementary to the target RNA, (2) the dRNA is capable of recruiting an adenosine deaminase acting on RNA (ADAR), and (3) the construct comprises a polymerase II promoter (“Pol II promoter”) operably linked to the nucleic acid encoding the dRNA.

In some embodiments, there are provided methods for editing a target RNA in a host cell, comprising introducing a construct comprising a nucleic acid encoding a deaminase-recruiting RNA (dRNA) into the host cell, wherein: (1) the dRNA comprises a targeting RNA sequence that is at least partially complementary to the target RNA, (2) the dRNA is capable of recruiting an endogenously expressed adenosine deaminase acting on RNA (ADAR) of the host cell to deaminate a target adenosine residue in the target RNA, and (3) the construct comprises a polymerase II promoter (“Pol I promoter”) operably linked to the nucleic acid encoding the dRNA. In some embodiments, the method does not comprise introducing any protein or construct comprising a nucleic acid encoding a protein (e.g., Cas, ADAR or a fusion protein of ADAR and Cas) to the host cell.

In some embodiments, provided herein are methods for editing a target RNA in a host cell, comprising introducing (a) a construct comprising a nucleic acid encoding a deaminase-recruiting RNA (dRNA) and (b) an ADAR or a construct comprising a nucleic acid encoding the ADAR into the host cell, wherein: (1) the dRNA comprises a targeting RNA sequence that is at least partially complementary to the target RNA, (2) the dRNA is capable of recruiting the adenosine deaminase acting on RNA (ADAR) to deaminate a target adenosine residue in the target RNA, and (3) the construct comprises a polymerase II promoter (“Pol II promoter”) operably linked to the nucleic acid encoding the dRNA. In some embodiments, the ADAR is an endogenously encoded ADAR of the host cell, wherein introduction of the ADAR comprises over-expressing the ADAR in the host cell. In some embodiments, the ADAR is exogenous to the host cell. In some embodiments, the construct comprising a nucleic acid encoding the ADAR is a vector, such as a plasmid, or a viral vector (e.g., an AAV or a lentiviral vector).

In one aspect, the present application provides a method for editing a plurality of target RNAs (e.g., at least about 2, 3, 4, 5, 10, 20, 50, 100, 1000 or more) in host cells by introducing a plurality of the dRNAs, or one or more constructs encoding the dRNAs, into the host cells. In some embodiments, one Pol II promoter (e.g., CMV) is driving expression of two or more dRNAs.

Non-limiting examples of Pol II promoters include: CMV, SV40, EF-1α, CAG and RSV. In some embodiments, the Pol II promoter is a CMV promoter. In some embodiments, the CMV promoter comprises the nucleic acid sequence of SEQ ID NO: 3 (5′-CGTTACATAACTTACGGTAAATGGCCCGCCTGGCTGACCGCCCAACGACCCCCGCCC ATGACGTCAATAATGACGTATGTTCCCATAGTAACGCCAATAGGGACFTCCATTG ACGTCAATGGGTGGAGTATTTACGGTAAACTGCCCACTTGGCAGTACATCAAGTGTA TCATATGCCAAGTACGCCCCCTATTGACGTCAATGACGGTAAATGGCCCGCCTGGCA TTATGCCCAGTACATGACCTTATGGGACFTTCCTACTTGGCAGTACATCTACGTATTA GTCATCGCTATTACCATGGTGATGCGGTTGGCAGTACATCAATGGGCGTGGATAG CGGTTTGACTCACGGGGATTTCCAAGTCTCCACCCCATTGACGTCAATGGGAGTTG TTTTGGCACCAAAATCAACGGGACTITCCAAAATGTCGTAACAACTCCGCCCCATTG ACGCAAATGGGCGGTAGGCGTGTACGGTGGGAGGTCTATATAAGCAGAGC′-3′).

In some embodiments according to any one of the methods described herein, the host cell is a prokaryotic cell. In some embodiments, the host cell is a eukaryotic cell. In some embodiments, the host cell is a mammalian cell. In some embodiments, the host cell is a human cell. In some embodiments, the host cell is a murine cell. In some embodiments, the host cell is a plant cell or a fungal cell.

In some embodiments according to any one of the methods or use described herein, the host cell is a cell line, such as HEK293T, HT29, A549, HepG2, RD, SF268, SW13 and HeLa cell. In some embodiments, the host cell is a primary cell, such as fibroblast, epithelial, or immune cell. In some embodiments, the host cell is a T cell. In some embodiments, the host cell is a post-mitosis cell. In some embodiments, the host cell is a cell of the central nervous system (CNS), such as a brain cell, e.g., a cerebellum cell.

In some embodiments according to any one of the methods described herein, the ADAR is endogenous to the host cell. In some embodiments, the adenosine deaminase acting on RNA (ADAR) is naturally or endogenously present in the host cell, for example, naturally or endogenously present in the eukaryotic cell. In some embodiments, the ADAR is endogenously expressed by the host cell. In certain embodiments, the ADAR is exogenously introduced into the host cell. In some embodiments, the ADAR is ADAR1 and/or ADAR2. In certain embodiments, the ADAR is one or more ADARs selected from the group consisting of hADAR1, hADAR2, mouse ADAR1 and ADAR2. In some embodiments, the ADAR is ADAR1, such as p110 isoform of ADAR1 (“ADAR1^{p110}”) and/or p150 isoform of ADAR1 (“ADAR1^{p150}”). In some embodiments, the ADAR is ADAR2. In some embodiments, the ADAR is an ADAR2 expressed by the host cell, e.g., ADAR2 expressed by cerebellum cells.

In some embodiments, the ADAR is an ADAR exogenous to the host cell. In some embodiments, the ADAR is a hyperactive mutant of a naturally occurring ADAR. In some embodiments, the ADAR is ADAR1 comprising an E1008Q mutation. In some embodiments, the ADAR is not a fusion protein comprising a binding domain. In some embodiments, the ADAR does not comprise an engineered double-strand nucleic acid-binding domain. In some embodiments, the ADAR does not comprise a MCP domain that binds to MS2 hairpin that is fused to the complementary RNA sequence in the dRNA. In some embodiments, the ADAR does not comprise a DSB.

In some embodiments according to any one of the methods described herein, the host cell has high expression level of ADAR1 (such as ADAR1^{p110} and/or ADAR1^{p150}), e.g., at least about any one of 10%, 20%, 50%, 100%, 2×, 3×, 5×, or more relative to the protein expression level of β-tubulin. In some embodiments, the host cell has high expression level of ADAR2, e.g., at least about any one of 10%, 20%, 50%, 100%, 2×, 3×, 5×, or more relative to the protein expression level of β-tubulin. In some embodiments, the host cell has low expression level of ADAR3, e.g., no more than about any one of 5×, 3×, 2×, 100%, 50%, 20% or less relative to the protein expression level of β-tubulin.

In certain embodiments according to any one of the methods described herein, the dRNA comprises at least about any one of 40, 45, 50, 55, 60, 65, 70, 75, 80, 90, 100, 110, 120, 130, 140, 150, 160, 170, 180, 190, 200, 210, 220, 230, 240, or 250 nucleotides. In certain embodiments according to any one of the methods described herein, the dRNA comprises no more than about any one of 40, 45, 50, 55, 60, 65, 70, 75, 80, 90, 100, 110, 120, 130, 140, 150, 160, 170, 180, 190, 200, 210, 220, 230, 240, or 250 nucleotides. In certain embodiments, the dRNA is about any one of 40-260, 45-250, 50-240, 60-230, 65-220, 70-220, 70-210, 70-200, 70-190, 70-180, 70-170, 70-160, 70-150, 70-140, 70-130, 70-120, 70-110, 70-100, 70-90, 70-80, 75-200, 80-190, 85-180, 90-170, 95-160, 100-200, 100-150, 100-175, 110-200, 110-175, 110-150, or 105-140 nucleotides in length.

In some embodiments according to any one of the methods or use described herein, the dRNA does not comprise an ADAR-recruiting domain. “ADAR-recruiting domain” can be a nucleotide sequence or structure that binds at high affinity to ADAR, or a nucleotide sequence that binds to a binding partner fused to ADAR in an engineered ADAR construct. Exemplary ADAR-recruiting domains include, but are not limited to, GluR-2, GluR-B (R/G), GluR-B (Q/R), GluR-6 (R/G), 5HT2C, and FlnA (Q/R) domain; see, for example, Wahlstedt, Helene, and Marie, “Site-selective versus promiscuous A-to-I editing.” Wiley Interdisciplinary Reviews: RNA 2.6 (2011): 761-771, which is incorporated herein by reference in its entirety. In some embodiments, the dRNA does not comprise a double-stranded portion. In some embodiments, the dRNA does not comprise a hairpin, such as MS2 stem loop. In some embodiments, the dRNA is single stranded. In some embodiments, the dRNA does not comprise a DSB-binding domain. In some embodiments, the dRNA consists of (or consists essentially of) the complementary RNA sequence.

In some embodiments according to any one of the methods described herein, the dRNA does not comprise chemical modifications. In some embodiments, the dRNA does not comprise a chemically modified nucleotide, such as 2′-O-methyl nucleotide or a nucleotide having a phosphorothioate linkage. In some embodiments, the dRNA comprises 2′-O-methyl and phosphorothioate linkage modifications only at the first three and last three residues. In some embodiments, the dRNA is not an antisense oligonucleotide (ASO).

The dRNAs described herein comprise a targeting RNA sequence that is at least partially complementary to the target RNA. In certain embodiments according to any one of the methods described herein, the targeting RNA sequence in the dRNA comprises at least about any one of 40, 45, 50, 55, 60, 65, 70, 75, 80, 90, 100, 110, 120, 130, 140, 150, 160, 170, 180, 190, 200, 210, 220, 230, 240, or 250 nucleotides. In certain embodiments according to any one of the methods described herein, the targeting RNA sequence in the dRNA comprises no more than about any one of 40, 45, 50, 55, 60, 65, 70, 75, 80, 90, 100, 110, 120, 130, 140, 150, 160, 170, 180, 190, 200, 210, 220, 230, 240, or 250 nucleotides. In certain embodiments, the targeting RNA sequence in the dRNA is about any one of 40-260, 45-250, 50-240, 60-230, 65-220, 70-220, 70-210, 70-200, 70-190, 70-180, 70-170, 70-160, 70-150, 70-140, 70-130, 70-120, 70-110, 70-100, 70-90, 70-80, 75-200, 80-190, 85-180, 90-170, 95-160, 100-200, 100-150, 100-175, 110-200, 110-160, 110-175, 110-150, 140-160, 105-140, or 105-155 nucleotides in length. In some embodiments, the targeting RNA sequence in the dRNA is about 71 nucleotides long. In some embodiments, the dRNA is about 111 nucleotides long. In some embodiments, the dRNA is about 151 nucleotides long.

In some embodiments, the targeting RNA sequence comprises a cytidine, adenosine or uridine directly opposite the target adenosine residue in the target RNA. In some embodiments, the targeting RNA sequence comprises a cytidine mismatch directly opposite the target adenosine residue in the target RNA. In some embodiments, the cytidine mismatch is located at least 5 nucleotides, e.g., at least 10, 15, 20, 25, 30, or more nucleotides, away from the 5′ end of the targeting RNA sequence. In some embodiments, the cytidine mismatch is located at least 20 nucleotides, e.g., at least 25, 30, 35, or more nucleotides, away from the 3′ end of the complementary RNA sequence. In some embodiments, the cytidine mismatch is not located within 20 (e.g., 15, 10, 5 or fewer) nucleotides away from the 3′ end of the targeting RNA sequence. In some embodiments, the cytidine mismatch is located at least 20 nucleotides (e.g., at least 25, 30, 35, or more nucleotides) away from the 3′ end and at least 5 nucleotides (e.g., at least 10, 15, 20, 25, 30, or more nucleotides) away from the 5′ end of the targeting RNA sequence. In some embodiments, the cytidine mismatch is located in the center of the targeting RNA sequence. In some embodiments, the cytidine mismatch is located within 20 nucleotides (e.g., 15, 10, 9, 8, 7, 6, 5, 4, 3, 2, or 1 nucleotide) of the center of the targeting sequence in the dRNA.

In some embodiments according to any one of the methods described herein, the targeting RNA sequence further comprises one or more guanosine(s), such as 1, 2, 3, 4, 5, 6, or more Gs, that is each directly opposite a non-target adenosine in the target RNA. In some embodiments, the targeting RNA sequence comprises two or more consecutive mismatch nucleotides (e.g., 2, 3, 4, 5, or more mismatch nucleotides) opposite a non-target adenosine in the target RNA.

In some embodiments, the target RNA comprises no more than about 20 non-target As, such as no more than about any one of 15, 10, 9, 8, 7, 6, 5, 4, 3, 2, or 1 non-target A. The Gs and consecutive mismatch nucleotides opposite non-target As may reduce off-target editing effects by ADAR.

In certain embodiments according to any one of the methods described herein, the 5′ nearest neighbor of the target adenosine residue is a nucleotide selected from U, C, A and G with the preference U>C≈A>G and the 3′ nearest neighbor of the target adenosine residue is a nucleotide selected from G, C, A and U with the preference G>C>A≈U. In certain embodiments, the target adenosine residue is in a three-base motif selected from the group consisting of UAG, UAC, UAA, UAU, CAG, CAC, CAA, CAU, AAG, AAC, AAA, AAU, GAG, GAC, GAA and GAU in the target RNA. In certain embodiments, the three-base motif is UAG, and the dRNA comprises an A directly opposite the U in the three-base motif, a C directly opposite the target A, and a C, G or U directly opposite the G in the three-base motif. In some embodiments, the three-base motif is UAG in the target RNA, and the dRNA comprises ACC, ACG or ACU that is opposite the UAG of the target RNA. In certain embodiments, the three-base motif is UAG in the target RNA, and the dRNA comprises ACC that is opposite the UAG of the target RNA.

In some embodiments according to any one of the methods described herein, the target RNA is any one selected from the group consisting of a pre-messenger RNA, a messenger RNA, a ribosomal RNA, a transfer RNA, a long non-coding RNA and a small RNA (e.g., miRNA). In some embodiments, the target RNA is a pre-messenger RNA. In some embodiments, the target RNA is a messenger RNA.

In certain embodiments according to any one of the methods described herein, the method further comprises introducing an inhibitor of ADAR3 to the host cell. In some embodiments, the inhibitor of ADAR3 is an RNAi against ADAR3, such as a shRNA against ADAR3 or a siRNA against ADAR3. In some embodiments, the method further comprises introducing a stimulator of interferon to the host cell. In some embodiments, the ADAR is inducible by interferon, for example, the ADAR is ADAR^{p150}. In some embodiments, the stimulator of interferon is IFNα. In some embodiments, the inhibitor of ADAR3 and/or the stimulator of interferon are encoded by the same construct (e.g., vector) that encodes the dRNA.

In certain embodiments according to any one of the methods described herein, the efficiency of editing of the target RNA is at least about 10%, such as at least about any one of 15%, 20%, 25%, 30%, 35%, 40%, 45%, 50%, 55%, 60%, 65%, 70%, 75%, 80%, 85%, 90%, or higher. In some embodiments, the efficiency of editing of the target RNA is at least about 40%. In some embodiments, the efficiency of editing is determined by Sanger sequencing. In some embodiments, the efficiency of editing is determined by next-generation sequencing. In some embodiments, the efficiency of editing is determined by assessing expression of a reporter gene, such as a fluorescence reporter, e.g., EGFP.

In certain embodiments according to any one of the methods described herein, the method has low off-target editing rate. In some embodiments, the method has lower than about 1% (e.g., no more than about any one of 0.5%, 0.1%, 0.05%, 0.01%, 0.001% or lower) editing efficiency on non-target As in the target RNA. In some embodiments, the method does not edit non-target As in the target RNA. In some embodiments, the method has lower than about 0.1% (e.g., no more than about any one of 0.05%, 0.01%, 0.005%, 0.001%, 0.0001% or lower) editing efficiency on As in non-target RNA.

In certain embodiments according to any one of the methods described herein, the method does not induce immune response, such as innate immune response. In some embodiments, the method does not induce interferon and/or interleukin expression in the host cell. In some embodiments, the method does not induce IFN-β and/or IL-6 expression in the host cell.

Also provided are edited RNA or host cells having an edited RNA produced by any one of the methods described herein. In some embodiments, the edited RNA comprises an inosine. In some embodiments, the host cell comprises an RNA having a missense mutation, an early stop codon, an alternative splice site, or an aberrant splice site. In some embodiments, the host cell comprises a mutant, truncated, or misfolded protein.

Methods of non-viral delivery of nucleic acids include lipofection, nucleofection, microinjection, biolistics, virosomes, liposomes, immunoliposomes, polycation or lipid: nucleic acid conjugates, electroporation, nanoparticles, exosomes, microvesicles, or gene-gun, naked DNA and artificial virions.

The use of RNA or DNA viral based systems for the delivery of nucleic acids has high efficiency in targeting a virus to specific cells and trafficking the viral payload to the cellular nuclei.

In certain embodiments according to any one of the methods described herein, the method comprises introducing a viral vector (such as an AAV or a lentiviral vector) encoding the dRNA to the host cell. In some embodiments, the vector is a recombinant adeno-associated virus (rAAV) vector. In some embodiments, the construct is flanked by one or more AAV inverted terminal repeat (ITR) sequences. In some embodiments, the construct is flanked by two AAV ITRs. In some embodiments, the AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV serotype ITRs. In some embodiments, the AAV ITRs are AAV2 ITRs. In some embodiments, the vector further comprises a stuffer nucleic acid. In some embodiments, the stuffer nucleic acid is located upstream or downstream of the nucleic acid encoding the dRNA. In some embodiments, the vector is a self-complementary rAAV vector. In some embodiments, the vector comprises first nucleic acid sequence encoding the dRNA and a second nucleic acid sequence encoding a complement of the dRNA, wherein the first nucleic acid sequence can form intrastrand base pairs with the second nucleic acid sequence along most or all of its length. In some embodiments, the first nucleic acid sequence and the second nucleic acid sequence are linked by a mutated AAV ITR, wherein the mutated AAV ITR comprises a deletion of the D region and comprises a mutation of the terminal resolution sequence. In some embodiments, the vector is encapsidated in a rAAV particle. In some embodiments, the AAV viral particle comprises an AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV2/2-7m8, AAV DJ, AAV2 N587A, AAV2 E548A, AAV2 N708A, AAV2 V708K, AAV2-HBKO, AAVDJ8, AAVPHP.B, AAVPHP.eB, AAVBR1, AAVHSC15, AAVHSC17, goat AAV, AAV1/AAV2 chimeric, bovine AAV, mouse AAV, or rAAV2/HBoV1 serotype capsid.

In some embodiments, the method comprises introducing a plasmid encoding the dRNA to the host cell. In some embodiments, the method comprises electroporation of the dRNA (e.g., synthetic dRNA) into the host cell. In some embodiments, the method comprises transfection of the dRNA into the host cell.

After deamination, modification of the target RNA and/or the protein encoded by the target RNA, can be determined using different methods depending on the positions of the targeted adenosines in the target RNA. For example, in order to determine whether “A” has been edited to “I” in the target RNA, RNA sequencing methods known in the art can be used to detect the modification of the RNA sequence. When the target adenosine is located in the coding region of an mRNA, the RNA editing may cause changes to the amino acid sequence encoded by the mRNA. For example, point mutations may be introduced to the mRNA of an innate or acquired point mutation in the mRNA may be reversed to yield wild-type gene product(s) because of the conversion of “A” to “I”. Amino acid sequencing by methods known in the art can be used to find any changes of amino acid residues in the encoded protein. Modifications of a stop codon may be determined by assessing the presence of a functional, elongated, truncated, full-length and/or wild-type protein. For example, when the target adenosine is located in a UGA, UAG, or UAA stop codon, modification of the target adenosine residue (UGA or UAG) or As (UAA) may create a read-through mutation and/or an elongated protein, or a truncated protein encoded by the target RNA may be reversed to create a functional, full-length and/or wild-type protein. Editing of a target RNA may also generate an aberrant splice site, and/or alternative splice site in the target RNA, thus leading to an elongated, truncated, or misfolded protein, or an aberrant splicing or alternative splicing site encoded in the target RNA may be reversed to create a functional, correctly-folding, full-length and/or wild-type protein. In some embodiments, the present application contemplates editing of both innate and acquired genetic changes, for example, missense mutation, early stop codon, aberrant splicing or alternative splicing site encoded by a target RNA. Using known methods to assess the function of the protein encoded by the target RNA can find out whether the RNA editing achieves the desired effects. Because deamination of the adenosine (A) to an inosine (I) may correct a mutated A at the target position in a mutant RNA encoding a protein, identification of the deamination into inosine may provide assessment on whether a functional protein is present, or whether a disease or drug resistance-associated RNA caused by the presence of a mutated adenosine is reversed or partly reversed. Similarly, because deamination of the adenosine (A) to an inosine (I) may introduce a point mutation in the resulting protein, identification of the deamination into inosine may provide a functional indication for identifying a cause of disease or a relevant factor of a disease.

When the presence of a target adenosine causes aberrant splicing, the read-out may be the assessment of occurrence and frequency of aberrant splicing. On the other hand, when the deamination of a target adenosine is desirable to introduce a splice site, then similar approaches can be used to check whether the required type of splicing occurs. An exemplary suitable method to identify the presence of an inosine after deamination of the target adenosine is RT-PCR and sequencing, using methods that are well-known to the person skilled in the art.

The effects of deamination of target adenosine(s) include, for example, point mutation, early stop codon, aberrant splice site, alternative splice site and misfolding of the resulting protein. These effects may induce structural and functional changes of RNAs and/or proteins associated with diseases, whether they are genetically inherited or caused by acquired genetic mutations, or may induce structural and functional changes of RNAs and/or proteins associated with occurrence of drug resistance. Hence, the dRNAs, the constructs encoding the dRNAs, and the RNA editing methods of present application can be used in prevention or treatment of hereditary genetic diseases or conditions, or diseases or conditions associated with acquired genetic mutations by changing the structure and/or function of the disease-associated RNAs and/or proteins.

In some embodiments, the target RNA is a regulatory RNA. In some embodiments, the target RNA to be edited is a ribosomal RNA, a transfer RNA, a long non-coding RNA or a small RNA (e.g., miRNA, pri-miRNA, pre-miRNA, piRNA, siRNA, snoRNA, snRNA, exRNA or scaRNA). The effects of deamination of the target adenosines include, for example, structural and functional changes of the ribosomal RNA, transfer RNA, long non-coding RNA or small RNA (e.g., miRNA), including changes of three-dimensional structure and/or loss of function or gain of function of the target RNA. In some embodiments, deamination of the target As in the target RNA changes the expression level of one or more downstream molecules (e.g., protein, RNA and/or metabolites) of the target RNA. Changes of the expression level of the downstream molecules can be increase or decrease in the expression level.

Some embodiments of the present application involve multiplex editing of target RNAs in host cells, which are useful for screening different variants of a target gene or different genes in the host cells. In some embodiments, wherein the method comprises introducing a plurality of dRNAs to the host cells, at least two of the dRNAs of the plurality of dRNAs have different sequences and/or have different target RNAs. In some embodiments, each dRNA has a different sequence and/or different target RNA. In some embodiments, the method generates a plurality (e.g., at least 2, 3, 5, 10, 50, 100, 1000 or more) of modifications in a single target RNA in the host cells. In some embodiments, the method generates a modification in a plurality (e.g., at least 2, 3, 5, 10, 50, 100, 1000 or more) of target RNAs in the host cells. In some embodiments, the method comprises editing a plurality of target RNAs in a plurality of populations of host cells. In some embodiments, each population of host cells receive a different dRNA or a dRNAs having a different target RNA from the other populations of host cells.

### III. DEAMINASE-RECRUITING RNA, CONSTRUCTS AND LIBRARIES

Also provided herein are deaminase-recruiting RNAs or constructs useful for any one of the methods described herein. Any one of the dRNAs or constructs described in this section may be used in the methods of RNA editing and treatment described herein. It is intended that any of the features and parameters described herein for dRNAs or constructs can be combined with each other, as if each and every combination is individually described. The dRNAs described herein do not comprise a tracrRNA, crRNA or gRNA used in a CRISPR/Cas system. In some embodiments, there is provided a deaminase-recruiting RNA (dRNA) for deamination of a target adenosine in a target RNA by recruiting an ADAR, comprising a complementary RNA sequence that hybridizes to the target RNA.

In one aspect, the present provides a construct comprising any one of the deaminase-recruiting RNAs described herein. In certain embodiments, the construct is a viral vector (such as a lentivirus vector) or a plasmid. In some embodiments, the construct encodes a single dRNA. In some embodiments, the construct encodes a plurality (e.g., about any one of 1, 2, 3, 4, 5, 10, 20 or more) dRNAs.

In one aspect, the present application provides a library comprising a plurality of the deaminase-recruiting RNAs or a plurality of the constructs described herein.

In one aspect, the present application provides a composition or a host cell comprising the deaminase-recruiting RNA or the construct described herein. In certain embodiments, the host cell is a prokaryotic cell or a eukaryotic cell. In some embodiments, the host cell is a mammalian cell. In some embodiments, the host cell is a human cell.

Circular dRNAs and Constructs

In one aspect, provided herein is a deaminase-recruiting RNAs (dRNA) for editing a target RNA in a host cell comprising a targeting RNA sequence that is at least partially complementary to the target RNA, wherein the dRNA is capable of recruiting an Adenosine Deaminase Acting on RNA (ADAR), and wherein the dRNA is circular or is capable of forming a circular RNA in the host cell.

In some embodiments, the dRNA is a linear RNA that is capable of forming a circular RNA in a host cell. In some embodiments, the dRNA is circulated by the Tornado method. In some embodiments, the dRNA transcript is also flanked by a 5′ and/or 3′ ligation sequences which are then flanked by the 5′-Twister ribozyme and/or 3′-Twister ribozymes, respectively. In some embodiments, the 3′ ligation sequence and the 5′ ligation sequence are at least partially complementary to each other. In some embodiments, the 3′ ligation sequence and the 5′ ligation sequence are at least about 50%, at least about 55%, at least about 60%, at least about 65%, at least about 70%, at least about 75%, at least about 80%, at least about 85%, at least about 90%, at least about 95%, at least about 96%, at least about 97%, at least about 98% or at least about 99% complementary to each other. In some embodiments, the 3′ ligation sequence and the 5′ ligation sequence are fully complementary to each other.

In some embodiments, the 3′ ligation sequence and the 5′ ligation sequence are independently at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides or at least about 100 nucleotides in length. In some embodiments, the 3′ ligation sequence and the 5′ ligation sequence are independently about 20-30 nucleotides, about 30-40 nucleotides, about 40-50 nucleotides, about 50-60 nucleotides, about 60-70 nucleotides, about 70-80 nucleotides, about 80-90 nucleotides, about 90-100 nucleotides, about 100-125 nucleotides, about 125-150 nucleotides, about 20-50 nucleotides, about 50-100 nucleotides or about 100-150 nucleotides in length.

In some embodiments, the dRNA is circularized by an RNA ligase. In some embodiments, the RNA ligase is expressed endogenously in the host cell. In some embodiments, the RNA ligase is RNA ligase RtcB. In some embodiments, the RNA ligase RtcB is expressed endogenously in the host cell. In some embodiments, the dRNA is circularized through in vitro enzymatic ligation (e.g., using RNA, DNA ligase, or splicing) or chemical ligation (e.g., using cyanogen bromide or a similar condensing agent).

Also provided herein is a construct comprising a nucleic acid encoding the dRNA. In some embodiments, the dRNA is introduced by a construct comprising a nucleic acid encoding the dRNA. In some embodiments, the construct further comprises a 3′ twister ribozyme sequence linked to the 3′ end of the nucleic acid encoding the dRNA. In some embodiments, the construct further comprises a 5′ twister ribozyme sequence linked to the 5′ end of the nucleic acid encoding the dRNA. In some embodiments, the construct further comprises a 3′ twister ribozyme sequence linked to the 3′ end of the nucleic acid encoding the dRNA and a 5′ twister ribozyme sequence linked to the 5′ end of the nucleic acid encoding the dRNA. In some embodiments, the 3′ twister sequence is twister P3 U2A and the 5′ twister sequence is twister P1. In some embodiments, wherein the 5′ twister sequence is twister P3 U2A and the 3′ twister sequence is twister P1. In some embodiments, the dRNA undergoes autocatalytic cleavage. In some embodiments, the catalyzed dRNA product comprises a 5′-hydroxyl group and a 2′,3′-cyclic phosphate at the 3′ terminus. In some embodiments, the catalyzed dRNA product is ligated by ubiquitous endogenous RNA ligase (e.g., RNA ligase RtcB). In some embodiments, the construct is a plasmid or a viral vector.

dRNA Having One or Two snoRNA Ends and Constructs

In another aspect, provided herein is a deaminase-recruiting RNA (dRNA) for editing a target RNA comprising: (1) a targeting RNA sequence that is at least partially complementary to the target RNA and (2) a small nucleolar RNA (snoRNA) sequence at the 3′ and/or 5′ ends of the targeting RNA sequence; wherein the dRNA is capable of recruiting an adenosine deaminase acting on RNA (ADAR).

In some embodiments, the dRNA comprises a snoRNA sequence linked to the 5′ end of the targeting RNA sequence (“5′ snoRNA sequence”). In some embodiments, the dRNA comprises a snoRNA sequence linked to the 3′ end of the targeting RNA sequence (“3′ snoRNA sequence”). In some embodiments, the snoRNA sequence is at least about 50 nucleotides, at least about 60 nucleotides, at least about 70 nucleotides, at least about 80 nucleotides, at least about 90 nucleotides, at least about 100 nucleotides, at least about 110 nucleotides, at least about 120 nucleotides, at least about 130 nucleotides, at least about 140 nucleotides, at least about 150 nucleotides, at least about 160 nucleotides, at least about 170 nucleotides, at least about 180 nucleotides, at least about 190 nucleotides or at least about 200 nucleotides in length.”). In some embodiments, the snoRNA sequence is about 50-75 nucleotides, about 75-100 nucleotides, about 100-125 nucleotides, about 125-150 nucleotides, about 150-175 nucleotides, about 175-200 nucleotides, about 50-100 nucleotides, about 100-150 nucleotides, about 150-200 nucleotides, about 125-175 nucleotides, or about 100-200 nucleotides in length.

In some embodiments, the 3′ snoRNA sequence comprises the nucleic acid sequence of SEQ ID NO: 1. In some embodiments, the 5′ snoRNA sequence comprises the nucleic acid sequence of SEQ ID NO: 2. In some embodiments, the snoRNA sequence is a C/D Box snoRNA sequence. In some embodiments, the snoRNA sequence is an H/ACA Box snoRNA sequence. In some embodiments, the snoRNA sequence is a composite C/D Box and H/ACA Box snoRNA sequence. In some embodiments, the snoRNA sequence is an orphan snoRNA sequence.

Constructs with Pol II Promoter

In another aspect, provided herein is a construct comprising a nucleic acid encoding a deaminase-recruiting RNA (dRNA) into the host cell, wherein: (1) the dRNA comprises a targeting RNA sequence that is at least partially complementary to the target RNA, (2) the dRNA is capable of recruiting an adenosine deaminase acting on RNA (ADAR), and (3) the construct comprises a polymerase II promoter (“Pol II promoter”) operably linked to the nucleic acid encoding the dRNA.

In some embodiments, the Pol II promoter that is operably linked to the coding nucleotide sequence, such that the promoter controls the transcription or expression of the coding nucleotide sequence. The Pol II promoter may be positioned 5′ (upstream) of a coding nucleotide sequence under its control. The distance between the Pol II promoter and the coding sequence may be approximately the same as the distance between that promoter and the gene it controls in the gene from which the promoter is derived. As is known in the art, variation in this distance may be accommodated without loss of promoter function. In some embodiments, the construct comprises a 5′ UTR and/or a 3′UTR that regulates the transcription or expression of the coding nucleotide sequence. In some embodiments, the Pol II promoter is a CMV promoter. In some embodiments, the CMV promoter comprises the nucleic acid sequence of SEQ ID NO: 3. In some embodiments, one Pol II promoter (e.g., CMV) is driving expression of two or more dRNAs.

In some embodiments according to any one of the dRNAs, constructs, libraries or compositions described herein, the targeting RNA sequence comprises a cytidine, adenosine or uridine directly opposite the target adenosine to be edited in the target RNA. In some embodiments, the targeting RNA sequence further comprises one or more guanosine(s) that is each directly opposite a non-target adenosine in the target RNA. In certain embodiments, the 5′ nearest neighbor of the target adenosine residue is a nucleotide selected from U, C, A and G with the preference U>C≈A>G and the 3′ nearest neighbor of the target adenosine residue is a nucleotide selected from G, C, A and U with the preference G>C>A≈U. In some embodiments, the 5′ nearest neighbor of the target adenosine residue is U. In some embodiments, the 5′ nearest neighbor of the target adenosine residue is C or A. In some embodiments, the 3′ nearest neighbor of the target adenosine residue is G. In some embodiments, the 3′ nearest neighbor of the target adenosine residue is C.

In some embodiments according to any one of the dRNAs, constructs, libraries or compositions described herein, the target adenosine residue is in a three-base motif selected from the group consisting of UAG, UAC, UAA, UAU, CAG, CAC, CAA, CAU, AAG, AAC, AAA, AAU, GAG, GAC, GAA and GAU in the target RNA. In some embodiments, the three-base motif is UAG, and the dRNA comprises an A directly opposite the U in the three-base motif, a C directly opposite the target A, and a C, G or U directly opposite the G in the three-base motif. In certain embodiments, the three-base motif is UAG in the target RNA, and the dRNA comprises ACC, ACG or ACU that is opposite the UAG of the target RNA.

In some embodiments, the dRNA comprises a cytidine mismatch directly opposite the target adenosine residue in the target RNA. In some embodiments, the cytidine mismatch is close to the center of the complementary RNA sequence, such as within 20, 15, 10, 5, 4, 3, 2, or 1 nucleotide away from the center of the complementary RNA sequence. In some embodiments, the cytidine mismatch is at least 5 nucleotides away from the 5′ end of the complementary RNA sequence. In some embodiments, the cytidine mismatch is at least 20 nucleotides away from the 3′ end of the complementary RNA sequence.

In some embodiments according to any one of the dRNAs, constructs, libraries or compositions described herein, the dRNA comprises more than about any one of 40, 45, 50, 55, 60, 65, 70, 75, 80, 90, 100, 110, 120, 130, 140, 150, 160, 170, 180, 190, 200, 210, 220, 230, 240, or 250 nucleotides. In certain embodiments, the dRNA is about any one of 40-260, 45-250, 50-240, 60-230, 65-220, 70-210, 70-200, 70-190, 70-180, 70-170, 70-160, 70-150, 70-140, 70-130, 70-120, 70-110, 70-100, 70-90, 70-80, 75-200, 80-190, 85-180, 90-170, 95-160, 100-150 or 105-140 nucleotides in length.

The dRNA of the present application comprises a targeting RNA sequence that hybridizes to the target RNA. The targeting RNA sequence is perfectly complementary or substantially complementarity to the target RNA to allow hybridization of the targeting RNA sequence to the target RNA. In some embodiments, the targeting RNA sequence has 100% sequence complementarity as the target RNA. In some embodiments, the targeting RNA sequence is at least about any one of 70%, 80%, 85%, 90%, 95%, 96%, 97%, 98%, or 99% or more complementary to over a continuous stretch of at least about any one of 20, 40, 60, 80, 100, 150, 200, or more nucleotides in the target RNA. In some embodiments, the dsRNA formed by hybridization between the targeting RNA sequence and the target RNA has one or more (e.g., 1, 2, 3, 4, 5, 6, 7, 8, 9, 10 or more) non-Watson-Crick base pairs (i.e., mismatches).

ADAR, for example, human ADAR enzymes edit double stranded RNA (dsRNA) structures with varying specificity, depending on a number of factors. One important factor is the degree of complementarity of the two strands making up the dsRNA sequence. Perfect complementarity of between the dRNA and the target RNA usually causes the catalytic domain of ADAR to deaminate adenosines in a non-discriminative manner. The specificity and efficiency of ADAR can be modified by introducing mismatches in the dsRNA region. For example, A-C mismatch is preferably recommended to increase the specificity and efficiency of deamination of the adenosine to be edited. Conversely, at the other A (adenosine) positions than the target adenosine residue (i.e., “non-target A”), the G-A mismatch can reduce off-target editing. Perfect complementarity is not necessarily required for a dsRNA formation between the dRNA and its target RNA, provided there is substantial complementarity for hybridization and formation of the dsRNA between the dRNA and the target RNA. In some embodiments, the dRNA sequence or single-stranded RNA region thereof has at least about any one of 70%, 80%, 85%, 90%, 95%, 96%, 97%, 98%, or 99% of sequence complementarity to the target RNA, when optimally aligned. Optimal alignment may be determined with the use of any suitable algorithm for aligning sequences, non-limiting examples of which include the Smith-Waterman algorithm, the Needleman-Wimsch algorithm, algorithms based on the Burrows-Wheeler Transform (e.g., the Burrows Wheeler Aligner).

The nucleotides neighboring the target adenosine also affect the specificity and efficiency of deamination. For example, the 5′ nearest neighbor of the target adenosine to be edited in the target RNA sequence has the preference U>C≈A>G and the 3′ nearest neighbor of the target adenosine to be edited in the target RNA sequence has the preference G>C>A≈U in terms of specificity and efficiency of deamination of adenosine. In some embodiments, when the target adenosine may be in a three-base motif selected from the group consisting of UAG, UAC, UAA, UAU, CAG, CAC, CAA, CAU, AAG, AAC, AAA, AAU, GAG, GAC, GAA and GAU in the target RNA, the specificity and efficiency of deamination of adenosine are higher than adenosines in other three-base motifs. In some embodiments, where the target adenosine to be edited is in the three-base motif UAG, UAC, UAA, UAU, CAG, CAC, AAG, AAC or AAA, the efficiency of deamination of adenosine is much higher than adenosines in other motifs. With respect to the same three-base motif, different designs of dRNA may also lead to different deamination efficiency. Taking the three-base motif UAG as an example, in some embodiments, when the dRNA comprises cytidine (C) directly opposite the target adenosine to be edited, adenosine (A) directly opposite the uridine, and cytidine (C), guanosine (G) or uridine (U) directly opposite the guanosine, the efficiency of deamination of the target adenosine is higher than that using other dRNA sequences. In some embodiments, when the dRNA comprises ACC, ACG or ACU opposite UAG of the target RNA, the editing efficiency of the A in the UAG of the target RNA may reach about 25%-90% (e.g., about 25%-80%, 25%-70%, 25%-60%, 25%-50%, 25%-40%, or 25%-30%).

Besides the target adenosines, there may be one or more adenosines in the target RNA, which are not desirable to be edited. With respect to these adenosines, it is preferable to reduce their editing efficiency as much as possible. It is found by this disclosure that where guanosine is directly opposite an adenosine in the target RNA, the deamination efficiency is significantly decreased. Therefore, in order to decrease off-target deamination, dRNAs can be designed to comprise one or more guanosines directly opposite one or more adenosine(s) other than the target adenosine to be edited in the target RNA.

The desired level of specificity and efficiency of editing the target RNA sequence may depend on different applications. Following the instructions in the present patent application, those of skill in the art will be capable of designing a dRNA having complementary or substantially complementary sequence to the target RNA sequence according to their needs, and, with some trial and error, obtain their desired results. As used herein, the term “mismatch” refers to opposing nucleotides in a double stranded RNA (dsRNA) which do not form perfect base pairs according to the Watson-Crick base pairing rules. Mismatch base pairs include, for example, G-A, C-A, U-C, A-A, G-G, C-C, U-U base pairs. Taking A-C match as an example, where a target adenosine residue is to be edited in the target RNA, a dRNA is designed to comprise a C opposite the A to be edited, generating an A-C mismatch in the dsRNA formed by hybridization between the target RNA and dRNA.

In some embodiments, the dsRNA formed by hybridization between the dRNA and the target RNA does not comprise a mismatch. In some embodiments, the dsRNA formed by hybridization between the dRNA and the target RNA comprises one or more, such as any one of 1, 2, 3, 4, 5, 6, 7 or more mismatches (e.g., the same type of different types of mismatches). In some embodiments, the dsRNA formed by hybridization between the dRNA and the target RNA comprises one or more kinds of mismatches, for example, 1, 2, 3, 4, 5, 6, 7 kinds of mismatches selected from the group consisting of G-A, C-A, U-C, A-A, G-G, C-C and U-U.

The mismatch nucleotides in the dsRNA formed by hybridization between the dRNA and the target RNA can form bulges, which can promote the efficiency of editing of the target RNA. There may be one (which is only formed at the target adenosine) or more bulges formed by the mismatches. The additional bulge-inducing mismatches may be upstream and/or downstream of the target adenosine. The bulges may be single-mismatch bulges (caused by one mismatching base pair) or multi-mismatch bulges (caused by more than one consecutive mismatching base pairs, e.g., two or three consecutive mismatching base pairs).

The targeting RNA sequence in the dRNA is single-stranded. The dRNA may be entirely single-stranded or have one or more (e.g., 1, 2, 3, or more) double-stranded regions and/or one or more stem loop regions. In some embodiments, the targeting RNA sequence is at least about any one of 40, 45, 50, 55, 60, 65, 70, 75, 80, 90, 100, 110, 120, 130, 140, 150, 160, 170, 180, 190, 200 or more nucleotides. In certain embodiments, the targeting RNA sequence is about any one of 40-260, 45-250, 50-240, 60-230, 65-220, 70-220, 70-210, 70-200, 70-190, 70-180, 70-170, 70-160, 70-150, 70-140, 70-130, 70-120, 70-110, 70-100, 70-90, 70-80, 75-200, 80-190, 85-180, 90-170, 95-160,100-200, 100-150, 100-175, 110-200, 110-160, 110-175, 110-150, 140-160, 105-140, or 105-155 nucleotides in length. In some embodiments, the targeting RNA sequence in the dRNA is about 71 nucleotides long. In some embodiments, the dRNA is about 111 nucleotides long. In some embodiments, the dRNA is about 151 nucleotides long.

In some embodiments, the dRNA, apart from the targeting RNA sequence, further comprises regions for stabilizing the dRNA, for example, one or more double-stranded regions and/or stem loop regions. In some embodiments, the double-stranded region or stem loop region of the dRNA comprises no more than about any one of 200, 150, 100, 50, 40, 30, 20, 10 or fewer base-pairs. In some embodiments, the dRNA does not comprise a stem loop or double-stranded region. In some embodiments, the dRNA comprises an ADAR-recruiting domain. In some embodiments, the dRNA does not comprise an ADAR-recruiting domain.

The dRNA may comprise one or more modifications. In some embodiments, the dRNA has one or more modified nucleotides, including nucleobase modification and/or backbone modification. Exemplary modifications to the RNA include, but are not limited to, phosphorothioate backbone modification, 2′-substitutions in the ribose (such as 2′-O-methyl and 2′-fluoro substitutions), LNA, and L-RNA. In some embodiments, the dRNA does not have modifications to the nucleobase or backbone.

The present application also contemplates a construct comprising the dRNA described herein, including, but not limited to, any of the constructs described in the sections above. The term “construct” as used herein refers to DNA or RNA molecules that comprise a coding nucleotide sequence that can be transcribed into RNAs or expressed into proteins. In some embodiments, the construct contains one or more regulatory elements operably linked to the nucleotide sequence encoding the RNA or protein. When the construct is introduced into a host cell, under suitable conditions, the coding nucleotide sequence in the construct can be transcribed or expressed.

In some embodiments, the construct comprises a promoter that is operably linked to the coding nucleotide sequence, such that the promoter controls the transcription or expression of the coding nucleotide sequence. A promoter may be positioned 5′ (upstream) of a coding nucleotide sequence under its control. The distance between the promoter and the coding sequence may be approximately the same as the distance between that promoter and the gene it controls in the gene from which the promoter is derived. As is known in the art, variation in this distance may be accommodated without loss of promoter function. In some embodiments, the construct comprises a 5′ UTR and/or a 3′UTR that regulates the transcription or expression of the coding nucleotide sequence. In some embodiments, the promoter is a U6 promoter. IN some embodiments, the promoter is a Poly II promoter as discussed in the sections described above.

In some embodiments, the construct is a vector encoding any one of the dRNAs disclosed in the present application. The term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the transcription or expression of coding nucleotide sequences to which they are operatively linked. Such vectors are referred to herein as “expression vectors”.

Recombinant expression vectors can comprise a nucleic acid of the present application in a form suitable for transcription or expression of the nucleic acid in a host cell. In some embodiments, the recombinant expression vector includes one or more regulatory elements, which may be selected on the basis of the host cells to be used for transcription or expression, which is operatively linked to the nucleic acid sequence to be transcribed or expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

In some embodiments, the vector is a rAAV vector. In some embodiments, the rAAV vector is a vector derived from an AAV serotype, including without limitation, AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV capsid serotype or the like. In some embodiments, the nucleic acid in the AAV comprises an ITR of AAV1, AAV2, AAV3, AAV4, AAV5, AAV6, AAV7, AAV8, AAVrh8, AAVrh8R, AAV9, AAV10, AAVrh0, AAV11, AAV12, AAV2R471A, AAV DJ, a goat AAV, bovine AAV, or mouse AAV capsid serotype or the like. In some embodiments, the nucleic acid in the AAV further encodes a dRNA as described herein. Use of any AAV serotype is considered within the scope of the present disclosure. In some embodiments, the vector is encapsidated in a rAAV particle. In some embodiments, the AAV viral particle comprises an AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV2/2-7m8, AAV DJ, AAV2 N587A, AAV2 E548A, AAV2 N708A, AAV2 V708K, AAV2-HBKO, AAVDJ8, AAVPHP.B, AAVPHP.eB, AAVBR1, AAVHSC15, AAVHSC17, goat AAV, AAV1/AAV2 chimeric, bovine AAV, mouse AAV, or rAAV2/HBoV1 serotype capsid.

In some embodiments, there is provided a construct (e.g., vector, such as viral vector) comprising a nucleotide sequence encoding the dRNA. In some embodiments, there is provided a construct (e.g., vector, such as viral vector) comprising a nucleotide sequence encoding the ADAR. In some embodiments, there is provided a construct comprising a first nucleotide sequence encoding the dRNA and a second nucleotide sequence encoding the ADAR. In some embodiments, the first nucleotide sequence and the second nucleotide sequence are operably linked to the same promoter. In some embodiments, the first nucleotide sequence and the second nucleotide sequence are operably linked to different promoters. In some embodiments, the promoter is inducible. In some embodiments, the construct does not encode for the ADAR. In some embodiments, the vector further comprises nucleic acid sequence(s) encoding an inhibitor of ADAR3 (e.g., ADAR3 shRNA or siRNA) and/or a stimulator of interferon (e.g., IFN-α).

### IV. METHODS OF TREATMENT

The RNA editing methods and compositions described herein may be used to treat or prevent a disease or condition in an individual, including, but not limited to hereditary genetic diseases and drug resistance.

In some embodiments, there is provided a method of editing a target RNA in a cell of an individual (e.g., human individual) ex vivo, comprising editing the target RNA using any one of the methods of RNA editing described herein.

In some embodiments, there is provided a method of editing a target RNA in a cell of an individual (e.g., human individual) ex vivo, comprising introducing a dRNA or a construct comprising a nucleic acid encoding the dRNA into the cell of the individual, wherein the dRNA comprises a targeting RNA sequence that hybridizes to the target RNA, wherein the dRNA is capable of recruiting an ADAR to deaminate a target adenosine residue in the target RNA, and wherein the dRNA is a circular RNA or capable of forming a circular RNA in the cell of the individual.

In some embodiments, there is provided a method of editing a target RNA in a cell of an individual (e.g., human individual) ex vivo, comprising introducing a dRNA or a construct comprising a nucleic acid encoding the dRNA into the cell of the individual, wherein the dRNA comprises a (1) targeting RNA sequence that hybridizes to the target RNA and (2) a small nucleolar RNA (snoRNA) sequence linked to the 3′ and/or 5′ ends of the targeting RNA sequence, and wherein the dRNA is capable of recruiting an ADAR to deaminate a target adenosine residue in the target RNA.

In some embodiments, there is provided a method of editing a target RNA in a cell of an individual (e.g., human individual) ex vivo, comprising introducing a construct comprising a nucleic acid encoding a dRNA into the cell of the individual, wherein the dRNA comprises a targeting RNA sequence that hybridizes to the target RNA, wherein the dRNA is capable of recruiting an ADAR to deaminate a target adenosine residue in the target RNA, and wherein the construct comprises a polymerase II promoter (“Pol II promoter”) operably linked to the nucleic acid encoding the dRNA. In some embodiments, the ADAR is endogenously expressed. In some embodiments, the method further comprises introducing the ADAR or a construct comprising a nucleic acid encoding the ADAR into the cell.

In some embodiments, the target RNA is associated with a disease or condition of the individual. In some embodiments, the disease or condition is a hereditary genetic disease, or a disease or condition associated with one or more acquired genetic mutations (e.g., drug resistance). In some embodiments, the method further comprises obtaining the cell from the individual.

In some embodiments, there is provided a method of treating or preventing a disease or condition in an individual (e.g., human individual), comprising editing a target RNA associated with the disease or condition in a cell of the individual using any one of the methods of RNA editing described herein.

In some embodiments, there is provided a method of treating or preventing a disease or condition in an individual (e.g., human individual), comprising introducing a dRNA or a construct comprising a nucleic acid encoding the dRNA into an isolated cell of the individual ex vivo, wherein the dRNA comprises a targeting RNA sequence that hybridizes to a target RNA associated with the disease or condition, wherein the dRNA is capable of recruiting an ADAR to deaminate a target adenosine residue in the target RNA and wherein the dRNA is a circular RNA or capable of forming a circular RNA in the isolated cell of the individual.

In some embodiments, there is provided a method of treating or preventing a disease or condition in an individual (e.g., human individual), comprising introducing a dRNA or a construct comprising a nucleic acid encoding the dRNA into an isolated cell of the individual ex vivo, wherein the dRNA comprises a (1) targeting RNA sequence that hybridizes to the target RNA and (2) a small nucleolar RNA (snoRNA) sequence linked to the 3′ and/or 5′ ends of the targeting RNA sequence, and wherein the dRNA is capable of recruiting an ADAR to deaminate a target adenosine residue in the target RNA.

In some embodiments, there is provided a method of treating or preventing a disease or condition in an individual (e.g., human individual), comprising introducing a construct comprising a nucleic acid encoding a dRNA into an isolated cell of the individual ex vivo, wherein the dRNA comprises a targeting RNA sequence that hybridizes to the target RNA, wherein the dRNA is capable of recruiting an ADAR to deaminate a target adenosine residue in the target RNA, and wherein the construct comprises a polymerase II promoter (“Pol II promoter”) operably linked to the nucleic acid encoding the dRNA.

In some embodiments, the ADAR is an endogenously expressed ADAR in the isolated cell. In some embodiments, the method comprises introducing the ADAR or a construct comprising a nucleic acid encoding the ADAR to the isolated cell. In some embodiments, the method further comprises culturing the cell having the edited RNA. In some embodiments, the method further comprises administering the cell having the edited RNA to the individual. In some embodiments, the disease or condition is a hereditary genetic disease, or a disease or condition associated with one or more acquired genetic mutations (e.g., drug resistance).

In some embodiments, there is provided a method of treating or preventing a disease or condition in an individual (e.g., human individual), comprising administering an effective amount of a dRNA or a construct comprising a nucleic acid encoding the dRNA to the individual, wherein the dRNA comprises a targeting RNA sequence that hybridizes to a target RNA associated with the disease or condition, wherein the dRNA is capable of recruiting an ADAR to deaminate a target adenosine residue in the target RNA and wherein the dRNA is a circular RNA or capable of forming a circular RNA in a cell of the individual.

In some embodiments, there is provided a method of treating or preventing a disease or condition in an individual (e.g., human individual), comprising administering an effective amount of a dRNA or a construct comprising a nucleic acid encoding the dRNA to the individual, wherein the dRNA comprises a (1) targeting RNA sequence that hybridizes to the target RNA and (2) a small nucleolar RNA (snoRNA) sequence linked to the 3′ and/or 5′ ends of the targeting RNA sequence, and wherein the dRNA is capable of recruiting an ADAR to deaminate a target adenosine residue in the target RNA.

In some embodiments, there is provided a method of treating or preventing a disease or condition in an individual (e.g., human individual), comprising administering an effective amount of a construct comprising a nucleic acid encoding a dRNA to the individual, wherein the dRNA comprises a targeting RNA sequence that hybridizes to the target RNA, wherein the dRNA is capable of recruiting an ADAR to deaminate a target adenosine residue in the target RNA, and wherein the construct comprises a polymerase II promoter (“Pol II promoter”) operably linked to the nucleic acid encoding the dRNA.

In some embodiments, the ADAR is an endogenously expressed ADAR in the cells of the individual. In some embodiments, the method comprises administering the ADAR or a construct comprising a nucleic acid encoding the ADAR to the individual. In some embodiments, the disease or condition is a hereditary genetic disease, or a disease or condition associated with one or more acquired genetic mutations (e.g., drug resistance).

Diseases and conditions suitable for treatment using the methods of the present application include diseases associated with a mutation, such as a G to A mutation, e.g., a G to A mutation that results in missense mutation, early stop codon, aberrant splicing, or alternative splicing in an RNA transcript. Examples of disease-associated mutations that may be restored by the methods of the present application include, but are not limited to, TP53^{W53X} (e.g., 158G>A) associated with cancer, IDUA^{W402X} (e.g., TGG>TAG mutation in exon 9) associated with Mucopolysaccharidosis type I (MPS I), COL3A1^{W1278X} (e.g., 3833G>A mutation) associated with Ehlers-Danlos syndrome, BMPR2^{W298X} (e.g., 893G>A) associated with primary pulmonary hypertension, AHI1^{W725X} (e.g., 2174G>A) associated with Joubert syndrome, FANCC^{W506X} (e.g., 1517G>A) associated with Fanconi anemia, MYBPC3^{W1098X} (e.g., 3293G>A) associated with primary familial hypertrophic cardiomyopathy, and IL2RG^{W237X} (e.g., 710G>A) associated with X-linked severe combined immunodeficiency. In some embodiments, the disease or condition is a cancer. In some embodiments, the disease or condition is a monogenetic disease. In some embodiments, the disease or condition is a polygenetic disease.

In some embodiments, there is provided a method of treating a cancer associated with a target RNA having a mutation (e.g., G>A mutation) in an individual, comprising introducing a dRNA or a construct comprising a nucleic acid encoding the dRNA into an isolated cell of the individual ex vivo, wherein the dRNA comprises a targeting RNA sequence that hybridizes to a target RNA associated with the disease or condition, wherein the dRNA is capable of recruiting an ADAR to deaminate a target adenosine residue in the target RNA and wherein the dRNA is a circular RNA or capable of forming a circular RNA in the isolated cell of the individual.

In some embodiments, there is provided a method of treating a cancer associated with a target RNA having a mutation (e.g., G>A mutation) in an individual, comprising introducing a dRNA or a construct comprising a nucleic acid encoding the dRNA into an isolated cell of the individual ex vivo, wherein the dRNA comprises a (1) targeting RNA sequence that hybridizes to the target RNA and (2) a small nucleolar RNA (snoRNA) sequence linked to the 3′ and/or 5′ ends of the targeting RNA sequence, and wherein the dRNA is capable of recruiting an ADAR to deaminate a target adenosine residue in the target RNA.

In some embodiments, there is provided a method of treating a cancer associated with a target RNA having a mutation (e.g., G>A mutation) in an individual, comprising introducing a construct comprising a nucleic acid encoding a dRNA into an isolated cell of the individual ex vivo, wherein the dRNA comprises a targeting RNA sequence that hybridizes to the target RNA, wherein the dRNA is capable of recruiting an ADAR to deaminate a target adenosine residue in the target RNA, and wherein the construct comprises a polymerase II promoter (“Pol II promoter”) operably linked to the nucleic acid encoding the dRNA.

In some embodiments, the ADAR is an endogenously expressed ADAR in the isolated cell. In some embodiments, the method comprises introducing the ADAR or a construct comprising a nucleic acid encoding the ADAR to the isolated cell. In some embodiments, the target RNA is TP53^{W53X} (e.g., 158G>A). In some embodiments, the dRNA comprises the nucleic acid sequence of SEQ ID NO: 4 (5′-GGGAGCAGCCUCUGGCAUUCUGGGAGCUUCAUCUGGACCUGGGUCUUCAGUGAAC CAUUGUUCAAUAUCGUCCGGGGACAGCAUCAAAUCAUCCAUUGCUUGGGACGGCA A-3′).

In some embodiments, there is provided a method of treating or preventing a cancer with a target RNA having a mutation (e.g., G>A mutation) in an individual, comprising administering an effective amount of a dRNA or a construct comprising a nucleic acid encoding the dRNA to the individual, wherein the dRNA comprises a targeting RNA sequence that hybridizes to a target RNA associated with the disease or condition, wherein the dRNA is capable of recruiting an ADAR to deaminate a target adenosine residue in the target RNA and wherein the dRNA is a circular RNA or capable of forming a circular RNA in a cell of the individual.

In some embodiments, there is provided a method of treating or preventing a cancer with a target RNA having a mutation (e.g., G>A mutation) in an individual, comprising administering an effective amount of a dRNA or a construct comprising a nucleic acid encoding the dRNA to the individual, wherein the dRNA comprises a (1) targeting RNA sequence that hybridizes to the target RNA and (2) a small nucleolar RNA (snoRNA) sequence linked to the 3′ and/or 5′ ends of the targeting RNA sequence, and wherein the dRNA is capable of recruiting an ADAR to deaminate a target adenosine residue in the target RNA.

In some embodiments, there is provided a method of treating or preventing a cancer with a target RNA having a mutation (e.g., G>A mutation) in an individual, comprising administering an effective amount of a construct comprising a nucleic acid encoding a dRNA to the individual, wherein the dRNA comprises a targeting RNA sequence that hybridizes to the target RNA, wherein the dRNA is capable of recruiting an ADAR to deaminate a target adenosine residue in the target RNA, and wherein the construct comprises a polymerase II promoter (“Pol II promoter”) operably linked to the nucleic acid encoding the dRNA.

In some embodiments, the ADAR is an endogenously expressed ADAR in the cells of the individual. In some embodiments, the method comprises administering the ADAR or a construct comprising a nucleic acid encoding the ADAR to the individual. In some embodiments, the target RNA is TP53^{W53X} (e.g., 158G>A). In some embodiments, the dRNA comprises the nucleic acid sequence of SEQ ID NO: 4.

In some embodiments, there is provided a method of treating MPS I (e.g., Hurler syndrome or Scheie syndrome) associated with a target RNA having a mutation (e.g., G>A mutation) in an individual, comprising introducing a dRNA or a construct comprising a nucleic acid encoding the dRNA into an isolated cell of the individual ex vivo, wherein the dRNA comprises a targeting RNA sequence that hybridizes to a target RNA associated with the disease or condition, wherein the dRNA is capable of recruiting an ADAR to deaminate a target adenosine residue in the target RNA and wherein the dRNA is a circular RNA or capable of forming a circular RNA in the isolated cell of the individual.

In some embodiments, there is provided a method of treating MPS I (e.g., Hurler syndrome or Scheie syndrome) associated with a target RNA having a mutation (e.g., G>A mutation) in an individual, comprising introducing a dRNA or a construct comprising a nucleic acid encoding the dRNA into an isolated cell of the individual ex vivo, wherein the dRNA comprises a (1) targeting RNA sequence that hybridizes to the target RNA and (2) a small nucleolar RNA (snoRNA) sequence linked to the 3′ and/or 5′ ends of the targeting RNA sequence, and wherein the dRNA is capable of recruiting an ADAR to deaminate a target adenosine residue in the target RNA.

In some embodiments, there is provided a method of treating MPS I (e.g., Hurler syndrome or Scheie syndrome) associated with a target RNA having a mutation (e.g., G>A mutation) in an individual, comprising introducing a construct comprising a nucleic acid encoding a dRNA into an isolated cell of the individual ex vivo, wherein the dRNA comprises a targeting RNA sequence that hybridizes to the target RNA, wherein the dRNA is capable of recruiting an ADAR to deaminate a target adenosine residue in the target RNA, and wherein the construct comprises a polymerase II promoter (“Pol II promoter”) operably linked to the nucleic acid encoding the dRNA.

In some embodiments, the ADAR is an endogenously expressed ADAR in the isolated cell. In some embodiments, the method comprises introducing the ADAR or a construct comprising a nucleic acid encoding the ADAR to the isolated cell. In some embodiments, the target RNA is IDUA^{W402X} (e.g., TGG>TAG mutation in exon 9). In some embodiments, the dRNA comprises the nucleic acid sequence of SEQ ID NO: 5 (5′-GACGCCCACCGUGUGGUUGCUGUCCAGGACGGUCCCGGCCUGCGACACUUCGGCC CAGAGCUGCUCCUCAUCUGCGGGGCGGGGGGGGGCCGUCGCCGCGUGGGGUCGUU G-3′).

In some embodiments, there is provided a method of treating or preventing MPS I (e.g., Hurler syndrome or Scheie syndrome) with a target RNA having a mutation (e.g., G>A mutation) in an individual, comprising administering an effective amount of a dRNA or a construct comprising a nucleic acid encoding the dRNA to the individual, wherein the dRNA comprises a targeting RNA sequence that hybridizes to a target RNA associated with the disease or condition, wherein the dRNA is capable of recruiting an ADAR to deaminate a target adenosine residue in the target RNA and wherein the dRNA is a circular RNA or capable of forming a circular RNA in a cell of the individual.

In some embodiments, there is provided a method of treating or preventing MPS I (e.g., Hurler syndrome or Scheie syndrome) with a target RNA having a mutation (e.g., G>A mutation) in an individual, comprising administering an effective amount of a dRNA or a construct comprising a nucleic acid encoding the dRNA to the individual, wherein the dRNA comprises a (1) targeting RNA sequence that hybridizes to the target RNA and (2) a small nucleolar RNA (snoRNA) sequence linked to the 3′ and/or 5′ ends of the targeting RNA sequence, and wherein the dRNA is capable of recruiting an ADAR to deaminate a target adenosine residue in the target RNA.

In some embodiments, there is provided a method of treating or preventing MPS I (e.g., Hurler syndrome or Scheie syndrome) with a target RNA having a mutation (e.g., G>A mutation) in an individual, comprising administering an effective amount of a construct comprising a nucleic acid encoding a dRNA to the individual, wherein the dRNA comprises a targeting RNA sequence that hybridizes to the target RNA, wherein the dRNA is capable of recruiting an ADAR to deaminate a target adenosine residue in the target RNA, and wherein the construct comprises a polymerase II promoter (“Pol II promoter”) operably linked to the nucleic acid encoding the dRNA.

In some embodiments, the ADAR is an endogenously expressed ADAR in the cells of the individual. In some embodiments, the method comprises administering the ADAR or a construct comprising a nucleic acid encoding the ADAR to the individual. In some embodiments, the target RNA is IDUA^{402X} (e.g., TGG>TAG mutation in exon 9). In some embodiments, the dRNA comprises the nucleic acid sequence of SEQ ID NO: 5.

In some embodiments, there is provided a method of treating a disease or condition Ehlers-Danlos syndrome associated with a target RNA having a mutation (e.g., G>A mutation) in an individual, comprising introducing a dRNA or a construct comprising a nucleic acid encoding the dRNA into an isolated cell of the individual ex vivo, wherein the dRNA comprises a targeting RNA sequence that hybridizes to a target RNA associated with the disease or condition, wherein the dRNA is capable of recruiting an ADAR to deaminate a target adenosine residue in the target RNA and wherein the dRNA is a circular RNA or capable of forming a circular RNA in the isolated cell of the individual.

In some embodiments, there is provided a method of treating a disease or condition Ehlers-Danlos syndrome associated with a target RNA having a mutation (e.g., G>A mutation) in an individual, comprising introducing a dRNA or a construct comprising a nucleic acid encoding the dRNA into an isolated cell of the individual ex vivo, wherein the dRNA comprises a (1) targeting RNA sequence that hybridizes to the target RNA and (2) a small nucleolar RNA (snoRNA) sequence linked to the 3′ and/or 5′ ends of the targeting RNA sequence, and wherein the dRNA is capable of recruiting an ADAR to deaminate a target adenosine residue in the target RNA.

In some embodiments, there is provided a method of treating a disease or condition Ehlers-Danlos syndrome associated with a target RNA having a mutation (e.g., G>A mutation) in an individual, comprising introducing a construct comprising a nucleic acid encoding a dRNA into an isolated cell of the individual ex vivo, wherein the dRNA comprises a targeting RNA sequence that hybridizes to the target RNA, wherein the dRNA is capable of recruiting an ADAR to deaminate a target adenosine residue in the target RNA, and wherein the construct comprises a polymerase II promoter (“Pol II promoter”) operably linked to the nucleic acid encoding the dRNA.

In some embodiments, the ADAR is an endogenously expressed ADAR in the isolated cell. In some embodiments, the method comprises introducing the ADAR or a construct comprising a nucleic acid encoding the ADAR to the isolated cell. In some embodiments, the target RNA is COL3A1^{W1278X} (e.g., 3833G>A mutation). In some embodiments, the dRNA comprises the nucleic acid sequence of SEQ ID NO: 6 (5′-CAUAUUACAGAAUACCUUGAUAGCAUCCAAUUUGCAUCCUUGGUUAGGGUCAAC CCAGUAUUCUCCACUCUUGAGUUCAGGAUGGCAGAAUUUCAGGUCUCUGCAGUU UCU-3′).

In some embodiments, there is provided a method of treating or preventing Ehlers-Danlos syndrome with a target RNA having a mutation (e.g., G>A mutation) in an individual, comprising administering an effective amount of a dRNA or a construct comprising a nucleic acid encoding the dRNA to the individual, wherein the dRNA comprises a targeting RNA sequence that hybridizes to a target RNA associated with the disease or condition, wherein the dRNA is capable of recruiting an ADAR to deaminate a target adenosine residue in the target RNA and wherein the dRNA is a circular RNA or capable of forming a circular RNA in a cell of the individual.

In some embodiments, there is provided a method of treating or preventing Ehlers-Danlos syndrome with a target RNA having a mutation (e.g., G>A mutation) in an individual, comprising administering an effective amount of a dRNA or a construct comprising a nucleic acid encoding the dRNA to the individual, wherein the dRNA comprises a (1) targeting RNA sequence that hybridizes to the target RNA and (2) a small nucleolar RNA (snoRNA) sequence linked to the 3′ and/or 5′ ends of the targeting RNA sequence, and wherein the dRNA is capable of recruiting an ADAR to deaminate a target adenosine residue in the target RNA.

In some embodiments, there is provided a method of treating or preventing Ehlers-Danlos syndrome with a target RNA having a mutation (e.g., G>A mutation) in an individual, comprising administering an effective amount of a construct comprising a nucleic acid encoding a dRNA to the individual, wherein the dRNA comprises a targeting RNA sequence that hybridizes to the target RNA, wherein the dRNA is capable of recruiting an ADAR to deaminate a target adenosine residue in the target RNA, and wherein the construct comprises a polymerase II promoter (“Pol II promoter”) operably linked to the nucleic acid encoding the dRNA.

In some embodiments, the ADAR is an endogenously expressed ADAR in the cells of the individual. In some embodiments, the method comprises administering the ADAR or a construct comprising a nucleic acid encoding the ADAR to the individual. In some embodiments, the target RNA is COL3A1^{W1278X} (e.g., 3833G>A mutation). In some embodiments, the dRNA comprises the nucleic acid sequence of SEQ ID NO: 6.

In some embodiments, there is provided a method of treating primary pulmonary hypertension associated with a target RNA having a mutation (e.g., G>A mutation) in an individual, comprising introducing a dRNA or a construct comprising a nucleic acid encoding the dRNA into an isolated cell of the individual ex vivo, wherein the dRNA comprises a targeting RNA sequence that hybridizes to a target RNA associated with the disease or condition, wherein the dRNA is capable of recruiting an ADAR to deaminate a target adenosine residue in the target RNA and wherein the dRNA is a circular RNA or capable of forming a circular RNA in the isolated cell of the individual.

In some embodiments, there is provided a method of treating primary pulmonary hypertension associated with a target RNA having a mutation (e.g., G>A mutation) in an individual, comprising introducing a dRNA or a construct comprising a nucleic acid encoding the dRNA into an isolated cell of the individual ex vivo, wherein the dRNA comprises a (1) targeting RNA sequence that hybridizes to the target RNA and (2) a small nucleolar RNA (snoRNA) sequence linked to the 3′ and/or 5′ ends of the targeting RNA sequence, and wherein the dRNA is capable of recruiting an ADAR to deaminate a target adenosine residue in the target RNA.

In some embodiments, there is provided a method of treating primary pulmonary hypertension associated with a target RNA having a mutation (e.g., G>A mutation) in an individual, comprising introducing a construct comprising a nucleic acid encoding a dRNA into an isolated cell of the individual ex vivo, wherein the dRNA comprises a targeting RNA sequence that hybridizes to the target RNA, wherein the dRNA is capable of recruiting an ADAR to deaminate a target adenosine residue in the target RNA, and wherein the construct comprises a polymerase II promoter (“Pol H promoter”) operably linked to the nucleic acid encoding the dRNA.

In some embodiments, the ADAR is an endogenously expressed ADAR in the isolated cell. In some embodiments, the method comprises introducing the ADAR or a construct comprising a nucleic acid encoding the ADAR to the isolated cell. In some embodiments, the target RNA is BMPR2^{W298X} (e.g., 893G>A). In some embodiments, the dRNA comprises the nucleic acid sequence of SEQ ID NO: 7 (5′-GUGAAGAUAAGCCAGUCCUCUAGUAACAGAAUGAGCAAGACGGCAAGAGCUUAC CCAGUCACUUGUGUGGAGACUUAAAUACUUGCAUAAAGAUCCAUUGGGAUAGUA CUC-3′).

In some embodiments, there is provided a method of treating or preventing primary pulmonary hypertension with a target RNA having a mutation (e.g., G>A mutation) in an individual, comprising administering an effective amount of a dRNA or a construct comprising a nucleic acid encoding the dRNA to the individual, wherein the dRNA comprises a targeting RNA sequence that hybridizes to a target RNA associated with the disease or condition, wherein the dRNA is capable of recruiting an ADAR to deaminate a target adenosine residue in the target RNA and wherein the dRNA is a circular RNA or capable of forming a circular RNA in a cell of the individual.

In some embodiments, there is provided a method of treating or preventing primary pulmonary hypertension with a target RNA having a mutation (e.g., G>A mutation) in an individual, comprising administering an effective amount of a dRNA or a construct comprising a nucleic acid encoding the dRNA to the individual, wherein the dRNA comprises a (1) targeting RNA sequence that hybridizes to the target RNA and (2) a small nucleolar RNA (snoRNA) sequence linked to the 3′ and/or 5′ ends of the targeting RNA sequence, and wherein the dRNA is capable of recruiting an ADAR to deaminate a target adenosine residue in the target RNA.

In some embodiments, there is provided a method of treating or preventing primary pulmonary hypertension with a target RNA having a mutation (e.g., G>A mutation) in an individual, comprising administering an effective amount of a dRNA to the individual, wherein the dRNA comprises a targeting RNA sequence that hybridizes to the target RNA, wherein the dRNA is capable of recruiting an ADAR to deaminate a target adenosine residue in the target RNA, and wherein the construct comprises a polymerase II promoter (“Pol II promoter”) operably linked to the nucleic acid encoding the dRNA.

In some embodiments, the ADAR is an endogenously expressed ADAR in the cells of the individual. In some embodiments, the method comprises administering the ADAR or a construct comprising a nucleic acid encoding the ADAR to the individual. In some embodiments, the target RNA is BMPR2^{W298X} (e.g., 893G>A). In some embodiments, the dRNA comprises the nucleic acid sequence of SEQ ID NO: 7.

In some embodiments, there is provided a method of treating Joubert syndrome associated with a target RNA having a mutation (e.g., G>A mutation) in an individual, comprising introducing a dRNA or a construct comprising a nucleic acid encoding the dRNA into an isolated cell of the individual ex vivo, wherein the dRNA comprises a targeting RNA sequence that hybridizes to a target RNA associated with the disease or condition, wherein the dRNA is capable of recruiting an ADAR to deaminate a target adenosine residue in the target RNA and wherein the dRNA is a circular RNA or capable of forming a circular RNA in the isolated cell of the individual.

In some embodiments, there is provided a method of treating Joubert syndrome associated with a target RNA having a mutation (e.g., G>A mutation) in an individual, comprising introducing a dRNA or a construct comprising a nucleic acid encoding the dRNA into an isolated cell of the individual ex vivo, wherein the dRNA comprises a (1) targeting RNA sequence that hybridizes to the target RNA and (2) a small nucleolar RNA (snoRNA) sequence linked to the 3′ and/or 5′ ends of the targeting RNA sequence, and wherein the dRNA is capable of recruiting an ADAR to deaminate a target adenosine residue in the target RNA.

In some embodiments, there is provided a method of treating Joubert syndrome associated with a target RNA having a mutation (e.g., G>A mutation) in an individual, comprising introducing a construct comprising a nucleic acid encoding a dRNA into an isolated cell of the individual ex vivo, wherein the dRNA comprises a targeting RNA sequence that hybridizes to the target RNA, wherein the dRNA is capable of recruiting an ADAR to deaminate a target adenosine residue in the target RNA, and wherein the construct comprises a polymerase II promoter (“Pol II promoter”) operably linked to the nucleic acid encoding the dRNA.

In some embodiments, the ADAR is an endogenously expressed ADAR in the isolated cell. In some embodiments, the method comprises introducing the ADAR or a construct comprising a nucleic acid encoding the ADAR to the isolated cell. In some embodiments, the target RNA is AHI1^{W725X} (e.g., 2174G>A). In some embodiments, the dRNA comprises the nucleic acid sequence of SEQ ID NO: 8 (5′-GUGAACGUCAAACUGUCGGACCAAUAUGGCAGAAUCUUCUCUCAUCUCAACUUUC CAUAUCCGUAUCAUGGAAUCAUAGCAUCCUGUAACUACUAGCUCUCUUACAGCUG G-3′).

In some embodiments, there is provided a method of treating or preventing Joubert syndrome with a target RNA having a mutation (e.g., G>A mutation) in an individual, comprising administering an effective amount of a dRNA or a construct comprising a nucleic acid encoding the dRNA to the individual, wherein the dRNA comprises a targeting RNA sequence that hybridizes to a target RNA associated with the disease or condition, wherein the dRNA is capable of recruiting an ADAR to deaminate a target adenosine residue in the target RNA and wherein the dRNA is a circular RNA or capable of forming a circular RNA in a cell of the individual.

In some embodiments, there is provided a method of treating or preventing Joubert syndrome with a target RNA having a mutation (e.g., G>A mutation) in an individual, comprising administering an effective amount of a dRNA or a construct comprising a nucleic acid encoding the dRNA to the individual, wherein the dRNA comprises a (1) targeting RNA sequence that hybridizes to the target RNA and (2) a small nucleolar RNA (snoRNA) sequence linked to the 3′ and/or 5′ ends of the targeting RNA sequence, and wherein the dRNA is capable of recruiting an ADAR to deaminate a target adenosine residue in the target RNA.

In some embodiments, there is provided a method of treating or preventing Joubert syndrome with a target RNA having a mutation (e.g., G>A mutation) in an individual, comprising administering an effective amount of a construct comprising a nucleic acid encoding a dRNA to the individual, wherein the dRNA comprises a targeting RNA sequence that hybridizes to the target RNA, wherein the dRNA is capable of recruiting an ADAR to deaminate a target adenosine residue in the target RNA, and wherein the construct comprises a polymerase II promoter (“Pol II promoter”) operably linked to the nucleic acid encoding the dRNA.

In some embodiments, the ADAR is an endogenously expressed ADAR in the cells of the individual. In some embodiments, the method comprises administering the ADAR or a construct comprising a nucleic acid encoding the ADAR to the individual. In some embodiments, the target RNA is AHI1^{W725X} (e.g., 2174G>A). In some embodiments, the dRNA comprises the nucleic acid sequence of SEQ ID NO: 8.

In some embodiments, there is provided a method of treating Fanconi anemia associated with a target RNA having a mutation (e.g., G>A mutation) in an individual, comprising introducing a dRNA or a construct comprising a nucleic acid encoding the dRNA into an isolated cell of the individual ex vivo, wherein the dRNA comprises a targeting RNA sequence that hybridizes to a target RNA associated with the disease or condition, wherein the dRNA is capable of recruiting an ADAR to deaminate a target adenosine residue in the target RNA and wherein the dRNA is a circular RNA or capable of forming a circular RNA in the isolated cell of the individual.

In some embodiments, there is provided a method of treating Fanconi anemia associated with a target RNA having a mutation (e.g., G>A mutation) in an individual, comprising introducing a dRNA or a construct comprising a nucleic acid encoding the dRNA into an isolated cell of the individual ex vivo, wherein the dRNA comprises a (1) targeting RNA sequence that hybridizes to the target RNA and (2) a small nucleolar RNA (snoRNA) sequence linked to the 3′ and/or 5′ ends of the targeting RNA sequence, and wherein the dRNA is capable of recruiting an ADAR to deaminate a target adenosine residue in the target RNA.

In some embodiments, there is provided a method of treating Fanconi anemia associated with a target RNA having a mutation (e.g., G>A mutation) in an individual, comprising introducing a construct comprising a nucleic acid encoding a dRNA into an isolated cell of the individual ex vivo, wherein the dRNA comprises a targeting RNA sequence that hybridizes to the target RNA, wherein the dRNA is capable of recruiting an ADAR to deaminate a target adenosine residue in the target RNA, and wherein the construct comprises a polymerase II promoter (“Pol II promoter”) operably linked to the nucleic acid encoding the dRNA.

In some embodiments, the ADAR is an endogenously expressed ADAR in the isolated cell. In some embodiments, the method comprises introducing the ADAR or a construct comprising a nucleic acid encoding the ADAR to the isolated cell. In some embodiments, the target RNA is FANCC^{W506X} (e.g., 1517G>A). In some embodiments, the dRNA comprises the nucleic acid sequence of SEQ ID NO: 9 (5′-GCCAAUGAUCUCGUGAGUUAUCUCAGCAGUGUGAGCCAUCAGGGUGAUGACAUC CCAGGCGAUCGUGUGGCCUCCAGGAGCCCAGAGCAGGAAGUUGAGGAGAAGGUG CCU-3′).

In some embodiments, there is provided a method of treating or preventing Fanconi anemia with a target RNA having a mutation (e.g., G>A mutation) in an individual, comprising administering an effective amount of a dRNA or a construct comprising a nucleic acid encoding the dRNA to the individual, wherein the dRNA comprises a targeting RNA sequence that hybridizes to a target RNA associated with the disease or condition, wherein the dRNA is capable of recruiting an ADAR to deaminate a target adenosine residue in the target RNA and wherein the dRNA is a circular RNA or capable of forming a circular RNA in a cell of the individual.

In some embodiments, there is provided a method of treating or preventing Fanconi anemia with a target RNA having a mutation (e.g., G>A mutation) in an individual, comprising administering an effective amount of a dRNA or a construct comprising a nucleic acid encoding the dRNA to the individual, wherein the dRNA comprises a (1) targeting RNA sequence that hybridizes to the target RNA and (2) a small nucleolar RNA (snoRNA) sequence linked to the 3′ and/or 5′ ends of the targeting RNA sequence, and wherein the dRNA is capable of recruiting an ADAR to deaminate a target adenosine residue in the target RNA.

In some embodiments, there is provided a method of treating or preventing Fanconi anemia with a target RNA having a mutation (e.g., G>A mutation) in an individual, comprising administering an effective amount of a construct comprising a nucleic acid encoding a dRNA to the individual, wherein the dRNA comprises a targeting RNA sequence that hybridizes to the target RNA, wherein the dRNA is capable of recruiting an ADAR to deaminate a target adenosine residue in the target RNA, and wherein the construct comprises a polymerase II promoter (“Pol 11 promoter”) operably linked to the nucleic acid encoding the dRNA.

In some embodiments, the ADAR is an endogenously expressed ADAR in the cells of the individual. In some embodiments, the method comprises administering the ADAR or a construct comprising a nucleic acid encoding the ADAR to the individual. In some embodiments, the target RNA is FANCC^{W506X} (e.g., 1517G>A). In some embodiments, the dRNA comprises the nucleic acid sequence of SEQ ID NO: 9.

In some embodiments, there is provided a method of treating primary familial hypertrophic cardiomyopathy associated with a target RNA having a mutation (e.g., G>A mutation) in an individual, comprising introducing a dRNA or a construct comprising a nucleic acid encoding the dRNA into an isolated cell of the individual ex vivo, wherein the dRNA comprises a targeting RNA sequence that hybridizes to a target RNA associated with the disease or condition, wherein the dRNA is capable of recruiting an ADAR to deaminate a target adenosine residue in the target RNA and wherein the dRNA is a circular RNA or capable of forming a circular RNA in the isolated cell of the individual.

In some embodiments, there is provided a method of treating primary familial hypertrophic cardiomyopathy associated with a target RNA having a mutation (e.g., G>A mutation) in an individual, comprising introducing a dRNA or a construct comprising a nucleic acid encoding the dRNA into an isolated cell of the individual ex vivo, wherein the dRNA comprises a (1) targeting RNA sequence that hybridizes to the target RNA and (2) a small nucleolar RNA (snoRNA) sequence linked to the 3′ and/or 5′ ends of the targeting RNA sequence, and wherein the dRNA is capable of recruiting an ADAR to deaminate a target adenosine residue in the target RNA.

In some embodiments, there is provided a method of treating primary familial hypertrophic cardiomyopathy associated with a target RNA having a mutation (e.g., G>A mutation) in an individual, comprising introducing a construct comprising a nucleic acid encoding a dRNA into an isolated cell of the individual ex vivo, wherein the dRNA comprises a targeting RNA sequence that hybridizes to the target RNA, wherein the dRNA is capable of recruiting an ADAR to deaminate a target adenosine residue in the target RNA, and wherein the construct comprises a polymerase II promoter (“Pol II promoter”) operably linked to the nucleic acid encoding the dRNA.

In some embodiments, the ADAR is an endogenously expressed ADAR in the isolated cell. In some embodiments, the method comprises introducing the ADAR or a construct comprising a nucleic acid encoding the ADAR to the isolated cell. In some embodiments, the target RNA is MYBPC3^{W1098X} (e.g., 3293G>A). In some embodiments, the dRNA comprises the nucleic acid sequence of SEQ ID NO: 10 (5′-CAAGACGGUGAACCACUCCAUGGUCUUCUUGUCGGCUUUCUGCACUGUGUACCCC CAGAGCUCCGUGUUGCCGACAUCCUGGGGUGGCUUCCACUCCAGAGCCACAUUAA G-3′).

In some embodiments, there is provided a method of treating or preventing primary familial hypertrophic cardiomyopathy with a target RNA having a mutation (e.g., G>A mutation) in an individual, comprising administering an effective amount of a dRNA or a construct comprising a nucleic acid encoding the dRNA to the individual, wherein the dRNA comprises a targeting RNA sequence that hybridizes to a target RNA associated with the disease or condition, wherein the dRNA is capable of recruiting an ADAR to deaminate a target adenosine residue in the target RNA and wherein the dRNA is a circular RNA or capable of forming a circular RNA in a cell of the individual.

In some embodiments, there is provided a method of treating or preventing primary familial hypertrophic cardiomyopathy with a target RNA having a mutation (e.g., G>A mutation) in an individual, comprising administering an effective amount of a dRNA or a construct comprising a nucleic acid encoding the dRNA to the individual, wherein the dRNA comprises a (1) targeting RNA sequence that hybridizes to the target RNA and (2) a small nucleolar RNA (snoRNA) sequence linked to the 3′ and/or 5′ ends of the targeting RNA sequence, and wherein the dRNA is capable of recruiting an ADAR to deaminate a target adenosine residue in the target RNA.

In some embodiments, there is provided a method of treating or preventing primary familial hypertrophic cardiomyopathy with a target RNA having a mutation (e.g., G>A mutation) in an individual, comprising administering an effective amount of a construct comprising a nucleic acid encoding a dRNA to the individual, wherein the dRNA comprises a targeting RNA sequence that hybridizes to the target RNA, wherein the dRNA is capable of recruiting an ADAR to deaminate a target adenosine residue in the target RNA, and wherein the construct comprises a polymerase II promoter (“Pol II promoter”) operably linked to the nucleic acid encoding the dRNA.

In some embodiments, the ADAR is an endogenously expressed ADAR in the cells of the individual. In some embodiments, the method comprises administering the ADAR or a construct comprising a nucleic acid encoding the ADAR to the individual. In some embodiments, the target RNA is MYBPC3^{W1098X} (e.g., 3293G>A). In some embodiments, the dRNA comprises the nucleic acid sequence of SEQ ID NO: 10.

In some embodiments, there is provided a method of treating X-linked severe combined immunodeficiency associated with a target RNA having a mutation (e.g., G>A mutation) in an individual, comprising introducing a dRNA or a construct comprising a nucleic acid encoding the dRNA into an isolated cell of the individual ex vivo, wherein the dRNA comprises a targeting RNA sequence that hybridizes to a target RNA associated with the disease or condition, wherein the dRNA is capable of recruiting an ADAR to deaminate a target adenosine residue in the target RNA and wherein the dRNA is a circular RNA or capable of forming a circular RNA in the isolated cell of the individual.

In some embodiments, there is provided a method of treating X-linked severe combined immunodeficiency associated with a target RNA having a mutation (e.g., G>A mutation) in an individual, comprising introducing a dRNA or a construct comprising a nucleic acid encoding the dRNA into an isolated cell of the individual ex vivo, wherein the dRNA comprises a (1) targeting RNA sequence that hybridizes to the target RNA and (2) a small nucleolar RNA (snoRNA) sequence linked to the 3′ and/or 5′ ends of the targeting RNA sequence, and wherein the dRNA is capable of recruiting an ADAR to deaminate a target adenosine residue in the target RNA.

In some embodiments, there is provided a method of treating X-linked severe combined immunodeficiency associated with a target RNA having a mutation (e.g., G>A mutation) in an individual, comprising introducing a construct comprising a nucleic acid encoding a dRNA into an isolated cell of the individual ex vivo, wherein the dRNA comprises a targeting RNA sequence that hybridizes to the target RNA, wherein the dRNA is capable of recruiting an ADAR to deaminate a target adenosine residue in the target RNA, and wherein the construct comprises a polymerase II promoter (“Pol I promoter”) operably linked to the nucleic acid encoding the dRNA.

In some embodiments, the ADAR is an endogenously expressed ADAR in the isolated cell. In some embodiments, the method comprises introducing the ADAR or a construct comprising a nucleic acid encoding the ADAR to the isolated cell. In some embodiments, the target RNA is IL2RG^{W237X} (e.g., 710G>A). In some embodiments, the dRNA comprises the nucleic acid sequence of SEQ ID NO: 11 (5′-AGGAUUCUCUUUUGAAGUAUUGCUCCCCCAGUGGAUUGGGUGGCUCCAUUCACUC CAAUGCUGAGCACUUCCACAGAGUGGGUUAAAGCGGCUCCGAACACGAAACGUGU A-3′).

In some embodiments, there is provided a method of treating or preventing X-linked severe combined immunodeficiency with a target RNA having a mutation (e.g., G>A mutation) in an individual, comprising administering an effective amount of a dRNA or a construct comprising a nucleic acid encoding the dRNA to the individual, wherein the dRNA comprises a targeting RNA sequence that hybridizes to a target RNA associated with the disease or condition, wherein the dRNA is capable of recruiting an ADAR to deaminate a target adenosine residue in the target RNA and wherein the dRNA is a circular RNA or capable of forming a circular RNA in a cell of the individual.

In some embodiments, there is provided a method of treating or preventing X-linked severe combined immunodeficiency with a target RNA having a mutation (e.g., G>A mutation) in an individual, comprising administering an effective amount of a dRNA or a construct comprising a nucleic acid encoding the dRNA to the individual, wherein the dRNA comprises a (1) targeting RNA sequence that hybridizes to the target RNA and (2) a small nucleolar RNA (snoRNA) sequence linked to the 3′ and/or 5′ ends of the targeting RNA sequence, and wherein the dRNA is capable of recruiting an ADAR to deaminate a target adenosine residue in the target RNA.

In some embodiments, there is provided a method of treating or preventing X-linked severe combined immunodeficiency with a target RNA having a mutation (e.g., G>A mutation) in an individual, comprising administering an effective amount of a construct comprising a nucleic acid encoding a dRNA to the individual, wherein the dRNA comprises a targeting RNA sequence that hybridizes to the target RNA, wherein the dRNA is capable of recruiting an ADAR to deaminate a target adenosine residue in the target RNA, and wherein the construct comprises a polymerase II promoter (“Pol II promoter”) operably linked to the nucleic acid encoding the dRNA.

In some embodiments, the ADAR is an endogenously expressed ADAR in the cells of the individual. In some embodiments, the method comprises administering the ADAR or a construct comprising a nucleic acid encoding the ADAR to the individual. In some embodiments, the target RNA is IL2RG^{W237X} (e.g., 710G>A). In some embodiments, the dRNA comprises the nucleic acid sequence of SEQ ID NO: 11.

Generally, dosages, schedules, and routes of administration of the compositions (e.g., dRNA or construct comprising a nucleic acid encoding dRNA) may be determined according to the size and condition of the individual, and according to standard pharmaceutical practice. Exemplary routes of administration include intravenous, intra-arterial, intraperitoneal, intrapulmonary, intravesicular, intramuscular, intra-tracheal, subcutaneous, intraocular, intrathecal, or transdermal.

The RNA editing methods of the present application not only can be used in animal cells, for example mammalian cells, but also may be used in modification of RNAs of plant or fungi, for example, in plants or fungi that have endogenously expressed ADARs. The methods described herein can be used to generate genetically engineered plant and fungi with improved properties.

The methods of treatment described herein may further comprise one or more steps of preparing a circular dRNA in vitro from a linear RNA precursor. Further provided are any one of the dRNAs, constructs, cells having edited RNA, and compositions described herein for use in any one of the methods of treatment described herein, and any one of the dRNAs, constructs, edited cells, and compositions described herein in the manufacture of a medicament for treating a disease or condition.

### V. COMPOSITIONS, KITS AND ARTICLES OF MANUFACTURE

Also provided herein are compositions (such as pharmaceutical compositions) comprising any one of the dRNAs, constructs, libraries, or host cells having edited RNA as described herein.

In some embodiments, there is provided a pharmaceutical composition comprising any one of the dRNAs or constructs encoding the dRNA described herein, and a pharmaceutically acceptable carriers, excipients or stabilizers (Remington's Pharmaceutical Sciences 16th edition, Osol, A. Ed. (1980)). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). In some embodiments, lyophilized formulations are provided. Pharmaceutical compositions to be used for in vivo administration must be sterile. This is readily accomplished by, e.g., filtration through sterile filtration membranes.

Further provided are kits useful for any one of the methods of RNA editing or methods of treatment described herein, comprising any one of the dRNAs, constructs, compositions, libraries, or edited host cells as described herein.

In some embodiments, there is provided a kit for editing a target RNA in a host cell, comprising a dRNA or a construct comprising a nucleic acid encoding the dRNA, wherein the dRNA comprises a targeting RNA sequence that hybridizes to a target RNA associated with the disease or condition, wherein the dRNA is capable of recruiting an ADAR to deaminate a target adenosine residue in the target RNA and wherein the dRNA is a circular RNA or capable of forming a circular RNA in the host cell.

In some embodiments, there is provided a kit for editing a target RNA in a host cell, comprising a dRNA or a construct comprising a nucleic acid encoding the dRNA, wherein the dRNA comprises a (1) targeting RNA sequence that hybridizes to the target RNA and (2) a small nucleolar RNA (snoRNA) sequence linked to the 3′ and/or 5′ ends of the targeting RNA sequence, and wherein the dRNA is capable of recruiting an ADAR to deaminate a target adenosine residue in the target RNA.

In some embodiments, there is provided a kit for editing a target RNA in a host cell, comprising a linear RNA or a construct thereof for preparing a circular dRNA in vitro, wherein the dRNA comprises a targeting RNA sequence that hybridizes to a target RNA associated with the disease or condition, wherein the dRNA is capable of recruiting an ADAR to deaminate a target adenosine residue in the target RNA in the host cell. In some embodiments, the kit further comprises instructions for preparing the circular dRNA in vitro, e.g., by splicing or by ligation. In some embodiments, the kit further comprises a ligase (e.g., T4 Rnl1 or T4Rnl2).

In some embodiments, there is provided a kit for editing a target RNA in a host cell, comprising or a construct comprising a nucleic acid encoding a dRNA, wherein the dRNA comprises a targeting RNA sequence that hybridizes to the target RNA, wherein the dRNA is capable of recruiting an ADAR to deaminate a target adenosine residue in the target RNA, and wherein the construct comprises a polymerase II promoter (“Pol H promoter”) operably linked to the nucleic acid encoding the dRNA.

In some embodiments, the kit further comprises an ADAR or a construct comprising a nucleic acid encoding an ADAR. In some embodiments, the kit further comprises an inhibitor of ADAR3 or a construct thereof. In some embodiments, the kit further comprises a stimulator of interferon or a construct thereof. In some embodiments, the kit further comprises an instruction for carrying out any one of the RNA editing methods or methods of treatment described herein.

The kits of the present application are in suitable packaging. Suitable packaging includes, but is not limited to, vials, bottles, jars, flexible packaging (e.g., sealed Mylar or plastic bags), and the like. Kits may optionally provide additional components such as transfection or transduction reagents, cell culturing medium, buffers, and interpretative information.

The present application thus also provides articles of manufacture. The article of manufacture can comprise a container and a label or package insert on or associated with the container. Suitable containers include vials (such as sealed vials), bottles, jars, flexible packaging, and the like. In some embodiments, the container holds a pharmaceutical composition, and may have a sterile access port (for example, the container may be an intravenous solution bag or a vial having a stopper pierceable by a hypodermic injection needle). The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical compositions and instructions for use, packaged in quantities sufficient for storage and use in pharmacies, for example, hospital pharmacies and compounding pharmacies.

The exemplary embodiments and examples below are intended to be purely exemplary of the present application and should therefore not be considered to limit the invention in any way. The following exemplary embodiments and examples and detailed description are offered by way of illustration and not by way of limitation.

### EXEMPLARY EMBODIMENTS

The present application provides the following embodiments:
1. A method for editing a target RNA in a host cell, comprising introducing a deaminase-recruiting RNA (dRNA) or a construct comprising a nucleic acid encoding the dRNA into the host cell, wherein:
   (1) the dRNA comprises a targeting RNA sequence that is at least partially complementary to the target RNA,
   (2) the dRNA is capable of recruiting an adenosine deaminase acting on RNA (ADAR), and
   (3) the dRNA is a circular RNA or capable of forming a circular RNA in the host cell.
2. The method of embodiment 1, wherein the dRNA further comprises a 3′ ligation sequence and a 5′ ligation sequence.
3. The method of embodiment 2, wherein the 3′ ligation sequence and the 5′ ligation sequence are at least partially complementary to each other.
4. The method of embodiment 2 or embodiment 3, wherein the 3′ ligation sequence and the 5′ ligation sequence are about 20 to about 75 nucleotides in length.
5. The method of any one of embodiments 1-4, wherein the dRNA is circularized by RNA ligase RtcB.
6. The method of embodiment 4, wherein the RNA ligase RtcB is expressed endogenously in the host cell.
7. The method of any one of embodiments 1-6, wherein the dRNA is a circular RNA (circRNA).
8. The method of any one of embodiments 1-6, wherein the dRNA is a linear RNA capable of forming a circular RNA in the host cell.
9. The method of any one of embodiments 1-6, wherein the method comprises introducing a construct comprising a nucleic acid encoding the dRNA into the host cell.
10. The method of claim 9, wherein the construct further comprises a 3′ twister ribozyme sequence linked to the 3′ end of the nucleic acid encoding the dRNA and a 5′ twister ribozyme sequence linked to the 5′ end of the nucleic acid encoding the dRNA.
11. The method of embodiment 10, wherein the 3′ twister sequence is twister P3 U2A and the 5′ twister sequence is twister P1.
12. The method of embodiment 10, wherein the 5′ twister sequence is twister P3 U2A and the 3′ twister sequence is twister P1.
13. The method of embodiment 7, further comprising forming the circular RNA using a linear RNA in vitro.
14. The method of embodiment 13, wherein the linear RNA is circularized by autocatalysis of a Group I intron comprising a 5′ catalytic Group I intron fragment and a 3′ catalytic Group I intron fragment.
15. A method for editing a target RNA in a host cell, comprising introducing a deaminase-recruiting RNA (dRNA) or a construct comprising a nucleic acid encoding the dRNA into the host cell, wherein:
   (1) the dRNA comprises a targeting RNA sequence that is at least partially complementary to the target RNA,
   (2) the dRNA is capable of recruiting an adenosine deaminase acting on RNA (ADAR), and
   (3) the dRNA is a circular RNA formed using a linear RNA in vitro by autocatalysis of a Group I intron comprising a 5′ catalytic Group I intron fragment and a 3′ catalytic Group I intron fragment.
16. The method of embodiment 14 or 15, wherein the linear RNA comprises the 3′ catalytic Group I intron fragment flanking the 5′ end of a 3′ exon sequence recognizable by the 3′ catalytic Group I intron fragment, and the 5′ catalytic Group I intron fragment flanking the 3′ end of a 5′ exon sequence recognizable by the 5′ catalytic Group I intron fragment.
17. The method of embodiment 16, wherein the linear RNA further comprises a 5′ homology sequence flanking the 5′ end of the 3′ catalytic Group I intron fragment, and a 3′ homology sequence flanking the 3′ end of the 5′ catalytic Group I intron fragment.
18. The method of any one of embodiments 14-17, wherein said forming a circular RNA comprises: (a) subjecting the linear RNA to a condition that activates autocatalysis of the 5′ catalytic Group I intron fragment and the 3′ catalytic Group I intron fragment to provide a circularized RNA product; and
   (b) isolating the circularized RNA product, thereby providing the circular RNA.
19. The method of embodiment 13, wherein the linear RNA is circularized by a ligase.
20. The method of embodiment 19, wherein the ligase is selected from the group consisting of a T4 DNA ligase (T4 Dnl), a T4 RNA ligase 1 (T4 Rnl1) and a T4 RNA ligase 2 (T4 Rnl2).
21. The method of embodiment 19 or 20, wherein the linear RNA comprises a 5′ ligation sequence at the 5′ end of a nucleic acid sequence encoding the circular RNA, and a 3′ ligation sequence at the 3′ end of the nucleic acid sequence encoding the circular RNA, wherein the 5′ ligation sequence and the 3′ ligation sequence can be ligated to each other via the ligase.
22. The method of embodiment 21, wherein said forming a circular RNA comprises:
   (a) contacting the linear RNA with a single-stranded adaptor nucleic acid comprising from the 5′ end to the 3′ end: a first sequence complementary to the 3′ ligation sequence and a second sequence complementary to the 5′ ligation sequence, and wherein the 5′ ligation sequence and the 3′ ligation sequence hybridize to the single-stranded adaptor nucleic acid to provide a duplex nucleic acid intermediate comprising a single strand break between the 3′ end of the 5′ ligation sequence and the 5′ end of the 3′ ligation sequence;
   (b) contacting the intermediate with an RNA ligase under a condition that allows ligation of the 5′ ligation sequence to the 3′ ligation sequence to provide a circularized RNA product; and
   (c) isolating the circularized RNA product, thereby providing the circular RNA.
23. The method of embodiment 21, wherein said forming a circular RNA comprises:
   (a) contacting the linear RNA with an RNA ligase under a condition that allows ligation of the 5′ ligation sequence to the 3′ ligation sequence to provide a circularized RNA product; and
   (b) isolating the circularized RNA product, thereby providing the circular RNA.
24. The method of any one of embodiments 13-23, further comprising obtaining the linear RNA by in vitro transcription of a nucleic acid construct comprising a nucleic acid sequence encoding the linear RNA, and/or purifying the circular RNA.
25. A method for editing a target RNA in a host cell, comprising introducing a deaminase-recruiting RNA (dRNA) or a construct comprising a nucleic acid encoding the dRNA into the host cell, wherein the dRNA comprises:
   (1) a targeting RNA sequence that is at least partially complementary to the target RNA and
   (2) a small nucleolar RNA (snoRNA) sequence linked to the 3′ and/or 5′ ends of the targeting RNA sequence;
and wherein the dRNA is capable of recruiting an adenosine deaminase acting on RNA (ADAR).
26. The method of embodiment 25, wherein the dRNA comprises a snoRNA sequence linked to the 5′ end of the targeting RNA sequence (“5′ snoRNA sequence”).
27. The method of embodiment 25 or 26, wherein the dRNA comprises a snoRNA sequence linked to the 3′ end of the targeting RNA sequence (3′ snoRNA sequence”).
28. The method of any one of embodiments 25-27, wherein the snoRNA sequence is at least about 70 nucleotides in length.
29. The method of any one of embodiments 25-28, wherein the 3′ snoRNA sequence comprises the nucleic acid sequence of SEQ ID NO: 1.
30. The method of any one of embodiments 25-29, wherein the 5′ snoRNA sequence comprises the nucleic acid sequence of SEQ ID NO: 2.
31. The method of any one of embodiments 25-30, wherein the snoRNA sequence is a C/D Box snoRNA sequence.
32. The method of any one of embodiments 25-30, wherein the snoRNA sequence is an H/ACA Box snoRNA sequence.
33. The method of any one of embodiments 25-30, wherein the snoRNA sequence is a composite C/D Box and H/ACA Box snoRNA sequence.
34. The method of any one of embodiments 25-30, wherein the snoRNA sequence is an orphan snoRNA sequence.
35. The method of any one of embodiments 25-34, wherein the method comprises introducing a construct comprising a nucleic acid encoding the dRNA into the host cell.
36. The method of embodiment any one of embodiments 9-12 and 25, wherein the construct further comprises a promoter operably linked to the nucleic acid encoding the dRNA.
37. The method of embodiment 36, wherein the promoter is a polymerase II promoter (“Pol 11 promoter”).
38. A method for editing a target RNA in a host cell, comprising introducing a construct comprising a nucleic acid encoding a deaminase-recruiting RNA (dRNA) into the host cell, wherein:
   (1) the dRNA comprises a targeting RNA sequence that is at least partially complementary to the target RNA,
   (2) the dRNA is capable of recruiting an adenosine deaminase acting on RNA (ADAR), and
   (3) the construct comprises a polymerase II promoter (“Pol II promoter”) operably linked to the nucleic acid encoding the dRNA.
39. The method of embodiment 37 or 38, wherein the Pol II promoter is a CMV promoter.
40. The method of embodiments 39, wherein the CMV promoter comprises the nucleic acid sequence of SEQ ID NO: 3.
41. The method of any one of embodiments 9-12 and 25-40, wherein the construct is a viral vector or a plasmid.
42. The method of embodiment 41, wherein the construct is an AAV vector.
43. The method of any one of embodiments 1-42, wherein the ADAR is endogenously expressed by the host cell.
44. The method of embodiment 43, wherein the host cell is a T cell.
45. The method of any one of embodiments 1-44, wherein the targeting RNA sequence is more than 50 nucleotides in length.
46. The method of embodiment 45, wherein the targeting RNA sequence is about 100 to about 150 nucleotides in length.
47. The method of any one of embodiments 1-46, wherein the targeting RNA sequence comprises a cytidine, adenosine or uridine directly opposite the target adenosine in the target RNA.
48. The method of embodiment 47, wherein the targeting RNA sequence comprises a cytidine mismatch directly opposite the target adenosine in the target RNA.
49. The method of embodiment 48, wherein the cytidine mismatch is located at least 20 nucleotides away from the 3′ end of the targeting RNA sequence, and at least 5 nucleotides away from the 5′ end of the targeting RNA sequence.
50. The method of any one of embodiments 1-49, wherein the targeting RNA sequence further comprises one or more guanosines each opposite a non-target adenosine in the target RNA.
51. The method of any one of embodiments 1-50, wherein the targeting RNA sequence comprises two or more consecutive mismatch nucleotides opposite a non-target adenosine in the target RNA.
52. The method of any one of embodiments 1-51, wherein the 5′ nearest neighbor of the target adenosine in the target RNA is a nucleotide selected from U, C, A and G with the preference U>C≈A>G and the 3′ nearest neighbor of the target adenosine in the target RNA is a nucleotide selected from G, C, A and U with the preference G>C>A≈U.
53. The method of any one of embodiments 1-52, wherein the target adenosine is in a three-base motif selected from the group consisting of UAG, UAC, UAA, UAU, CAG, CAC, CAA, CAU, AAG, AAC, AAA, AAU, GAG, GAC, GAA and GAU in the target RNA.
54. The method of embodiment 53, wherein the three-base motif is UAG, and wherein the targeting RNA comprises an A directly opposite the uridine in the three-base motif, a cytidine directly opposite the target adenosine, and a cytidine, guanosine or uridine directly opposite the guanosine in the three-base motif.
55. The method of any one of embodiments 1-54, wherein the target RNA is an RNA selected from the group consisting of a pre-messenger RNA, a messenger RNA, a ribosomal RNA, a transfer RNA, a long non-coding RNA and a small RNA.
56. The method of embodiment 55, wherein the target RNA is a pre-messenger RNA.
57. The method of any one of embodiments 1-56, further comprising introducing an inhibitor of ADAR3 and/or to the host cell.
58. The method of any one of embodiments 1-57, further comprising introducing a stimulator of interferon to the host cell.
59. The method of any one of embodiments 1-58, comprising introducing a plurality of dRNAs or constructs each targeting a different target RNA.
60. The method of any one of embodiments 1-59, wherein the efficiency of editing the target RNA is at least 40%.
61. The method of any one of embodiments 1-60, wherein the construct or the dRNA does not induce immune response.
62. The method of any one of embodiments 1-61, further comprising introducing an ADAR (e.g., exogenous ADAR) to the host cell.
63. The method of embodiment 62, wherein the ADAR is an ADAR1 comprising an E1008 mutation.
64. The method of any one of embodiments 1-63, wherein deamination of the target adenosine in the target RNA results in a missense mutation, an early stop codon, aberrant splicing, or alternative splicing in the target RNA, or reversal of a missense mutation, an early stop codon, aberrant splicing, or alternative splicing in the target RNA.
65. The method of embodiment 64, wherein deamination of the target adenosine in the target RNA results in point mutation, truncation, elongation and/or misfolding of the protein encoded by the target RNA, or a functional, full-length, correctly-folded and/or wild-type protein by reversal of a missense mutation, an early stop codon, aberrant splicing, or alternative splicing in the target RNA.
66. The method of any one of embodiments 1-65, wherein the host cell is a eukaryotic cell.
67. The method of embodiment 66, wherein the host cell is a mammalian cell.
68. The method of embodiment 67, wherein the host cell is a human or mouse cell.
69. An edited RNA or a host cell having an edited RNA produced by the method of any one of embodiments 1-68.
70. A method for treating or preventing a disease or condition in an individual, comprising editing a target RNA associated with the disease or condition in a cell of the individual according to the method of any one of the embodiments 1-69.
71. The method of embodiment 70, wherein the disease or condition is a hereditary genetic disease or a disease or condition associated with one or more acquired genetic mutations.
72. The method of embodiment 70 or 71, wherein the target RNA has a G to A mutation.
73. The method of any one of embodiments 70-72, wherein disease or condition is a monogenetic disease or condition.
74. The method of any one of embodiments 70-73, wherein the disease or condition is a polygenetic disease or condition.
75. The method of any one of embodiments 70-74, wherein:
   (i) the target RNA is TP53, and the disease or condition is cancer;
   (ii) the target RNA is IDUA, and the disease or condition is Mucopolysaccharidosis type 1 (MPS 1);
   (iii) the target RNA is COL3A1, and the disease or condition is Ehlers-Danlos syndrome;
   (iv) the target RNA is BMPR2, and the disease or condition is Joubert syndrome;
   (v) the target RNA is FANCC, and the disease or condition is Fanconi anemia;
   (vi) the target RNA is MYBPC3, and the disease or condition is primary familial hypertrophic cardiomyopathy; or
   (vii) the target RNA is IL2RG, and the disease or condition is X-linked severe combined immunodeficiency.
76. A deaminase-recruiting RNA (dRNA) for editing a target RNA in a host cell comprising a targeting RNA sequence that is at least partially complementary to the target RNA, wherein the dRNA is capable of recruiting an Adenosine Deaminase Acting on RNA (ADAR), and wherein the dRNA is circular or is capable of forming a circular RNA in the host cell.
77. The dRNA of embodiment 76, wherein the dRNA further comprises a 3′ ligation sequence and a 5′ ligation sequence.
78. The dRNA of embodiment 77, wherein the 3′ ligation sequence and the 5′ ligation sequence are at least partially complementary to each other.
79. The dRNA of embodiment 77 or 78, wherein the 3′ ligation sequence and the 5′ ligation sequence are about 20 to about 75 nucleotides in length.
80. The dRNA of embodiment 76, wherein the dRNA is a circular RNA.
81. The dRNA of embodiment 80, wherein the dRNA is a circular RNA formed from a linear RNA in vitro.
82. The dRNA of any one of embodiments 76-79, wherein the dRNA is a linear RNA capable of forming a circular RNA in the host cell.
83. The dRNA of embodiment 81, wherein the linear RNA is circularized by autocatalysis of a Group I intron comprising a 5′ catalytic Group I intron fragment and a 3′ catalytic Group I intron fragment.
84. The dRNA of embodiment 83, wherein the linear RNA comprises the 3′ catalytic Group I intron fragment flanking the 5′ end of a 3′ exon sequence recognizable by the 3′ catalytic Group I intron fragment, and the 5′ catalytic Group I intron fragment flanking the 3′ end of a 5′ exon sequence recognizable by the 5′ catalytic Group I intron fragment.
85. The dRNA of embodiment 84, wherein the linear RNA further comprises a 5′ homology sequence flanking the 5′ end of the 3′ catalytic Group I intron fragment, and a 3′ homology sequence flanking the 3′ end of the 5′ catalytic Group I intron fragment.
86. The dRNA of embodiment 82, wherein the linear RNA is circularized by a ligase.
87. The dRNA of embodiment 86, wherein the ligase is selected from the group consisting of a T4 DNA ligase (T4 Dnl), a T4 RNA ligase 1 (T4 Rnl1) and a T4 RNA ligase 2 (T4 Rnl2).
88. The dRNA of embodiment 87, wherein the linear RNA comprises a 5′ ligation sequence at the 5′ end of the nucleic acid sequence encoding the circular dRNA, and a 3′ ligation sequence at the 3′ end of the nucleic acid sequence encoding the circular dRNA, wherein the 5′ ligation sequence and the 3′ ligation sequence can be ligated to each other via the RNA ligase.
89. A linear RNA capable of forming the circular dRNA of any one of embodiments 83-88.
90. A construct comprising a nucleic acid encoding the dRNA of any one of embodiments 76-88 or the linear RNA of embodiment 89.
91. The construct of embodiment 90, wherein the construct further comprises a 3′ twister ribozyme sequence linked to the 3′ end of the nucleic acid encoding the dRNA and a 5′ twister ribozyme sequence linked to the 5′ end of the nucleic acid encoding the dRNA.
92. The construct of embodiment 91, wherein the 3′ twister sequence is twister P3 U2A and the 5′ twister sequence is twister P1.
93. The construct of embodiment 92, wherein the 5′ twister sequence is twister P3 U2A and the 3′ twister sequence is twister P1.
94. A deaminase-recruiting RNA (dRNA) for editing a target RNA comprising:
   (1) a targeting RNA sequence that is at least partially complementary to the target RNA and
   (2) a small nucleolar RNA (snoRNA) sequence at the 3′ and/or 5′ ends of the targeting RNA sequence;
wherein the dRNA is capable of recruiting an adenosine deaminase acting on RNA (ADAR).
95. The dRNA of embodiment 94, wherein the dRNA comprises a snoRNA sequence linked to the 5′ end of the targeting RNA sequence (“5′ snoRNA sequence”).
96. The dRNA of embodiment 95, wherein the dRNA comprises a snoRNA sequence linked to the 3′ end of the targeting RNA sequence (3′ snoRNA sequence”).
97. The dRNA of any one of embodiments 94-96, wherein the snoRNA sequence is at least about 70 nucleotides in length.
98. The dRNA of any one of embodiments 96-97, wherein the 3′ snoRNA sequence comprises the nucleic acid sequence of SEQ ID NO: 1.
99. The dRNA of any one of embodiments 95-98, wherein the 5′ snoRNA sequence comprises the nucleic acid sequence of SEQ ID NO: 2.
100. The dRNA of any one of embodiments 94-99, wherein the snoRNA sequence is a CID Box snoRNA sequence.
101. The dRNA of anyone of embodiments 94-99, wherein the snoRNA sequence is an H/ACA Box snoRNA sequence.
102. The dRNA of any one of embodiments 94-99, wherein the snoRNA sequence is a composite C/D Box and H/ACA Box snoRNA sequence.
103. The dRNA of any one of embodiments 94-99, wherein the snoRNA sequence is an orphan snoRNA sequence.
104. A construct comprising a nucleic acid encoding a dRNA of any one of embodiments 94-103.
95. The construct of any one of embodiments 90-93 and 104, wherein the construct further comprises a promoter operably linked to the nucleic acid encoding the dRNA.
106. The construct of embodiment 10⁵, wherein the promoter is a polymerase II promoter (“Pol II promoter”).
107. A construct comprising a nucleic acid encoding a deaminase-recruiting RNA (dRNA) into the host cell, wherein:
   (1) the dRNA comprises a targeting RNA sequence that is at least partially complementary to the target RNA,
   (2) the dRNA is capable of recruiting an adenosine deaminase acting on RNA (ADAR), and
   (3) the construct comprises a polymerase II promoter (“Pol II promoter”) operably linked to the nucleic acid encoding the dRNA.
108. The construct of embodiment 106 or 107, wherein the Pol II promoter is a CMV promoter.
109. The construct of embodiment 108, wherein the CMV promoter comprises the nucleic acid sequence of SEQ ID NO: 3.
110. The construct of any one of embodiments 90-93 and 104-109, wherein the construct is a viral vector or a plasmid.
111. The construct of embodiment 110, wherein the construct is an AAV vector.
112. The construct or dRNA of any one of embodiments 76-88 and 90-111, wherein the target RNA is an RNA selected from the group consisting of a pre-messenger RNA, a messenger RNA, a ribosomal RNA, a transfer RNA, a long non-coding RNA and a small RNA.
113. A host cell comprising the construct or dRNA of any one of embodiments 76-88 and 90-111.
114. A kit for editing a target RNA in a host cell comprising the construct, linear RNA or dRNA of any one of embodiments 76-111.

### Example

### Materials and Methods

### Plasmids Construction

The dual fluorescence reporter was cloned by PCR amplifying mCherry and EGFP (the EGFP first codon ATG was deleted) coding DNA, the 3×GS linker and targeting DNA sequence were added via primers during PCR. Then the PCR products were cleaved and linked by Type IIs restriction enzyme BsmB1 (Thermo) and T4 DNA ligase (NEB), which then were inserted into pLenti backbone (pLenti-CMV-MCS-SV-Bsd, Stanley Cohen Lab, Stanford University).

The dLbuCas13 DNA was PCR amplified from the Lbu plasmids (Addgene #83485). The ADAR1DD and ADAR2DD were amplified from Adar1(p150) cDNA and Adar2 cDNA, both of which were gifts from Han's lab at Xiamen University. The ADAR1DD or ADAR2DD were fused to dLbuCas13 DNA by overlap-PCR, and the fused PCR products were inserted into pLenti backbone.

For expression of dRNA in mammalian cells, the dRNA sequences were directly synthesized (for short dRNAs) and annealed or PCR amplified by synthesizing overlapping ssDNA, and the products were cloned into the corresponding vectors under U6 expression by Golden-gate cloning.

The full length Adar1(p110) and Adar1(p150) were PCR amplified from Adar1(p150) cDNA, and the full length Adar2 were PCR amplified from Adar2 cDNA, which were then cloned into pLenti backbone, respectively.

For the two versions of dual fluorescence reporters (Reporter-1 and -3), mCherry and EGFP (the start codon ATG of EGFP was deleted) coding sequences were PCR amplified, digested using BsmBI (Thermo Fisher Scientific, ER0452), followed by T4 DNA ligase (NEB, M0202L)-mediated ligation with GGGGS linkers. The ligation product was subsequently inserted into the pLenti-CMV-MCS-PURO backbone.

For the dLbuCas13-ADAR_{DD} (E1008Q) expressing construct, the ADAR1_{DD} gene was amplified from the ADAR^{p150} construct (a gift from Jiahuai Han's lab, Xiamen University). The dLbuCas13 gene was amplified by PCR from the Lbu_C2c2_R472A_H477A_R1048A_H1053A plasmid (Addgene #83485). The ADAR1_{DD} (hyperactive E1008Q variant) was generated by overlap-PCR and then fused to dLbuCas13. The ligation products were inserted into the pLenti-CMV-MCS-BSD backbone.

For arRNA-expressing construct, the sequences of arRNAs were synthesized and golden-gate cloned into the pLenti-sgRNA-lib 2.0 (Addgene #89638) backbone, and the transcription of arRNA was driven by hU6 promoter. For the ADAR expressing constructs, the full length ADAR1^{p110} and ADAR1^{p150} were PCR amplified from the ADAR1^{p150} construct, and the full length ADAR2 were PCR amplified from the ADAR2 construct (a gift from Jiahuai Han's lab, Xiamen University). The amplified products were then cloned into the pLenti-CMV-MCS-BSD backbone.

For the constructs expressing genes with pathogenic mutations, full length coding sequences of TP53 (ordered from Vigenebio) and other 6 disease-relevant genes (COL3A1, BMPR2, AHI1, FANCC, MYBPC3 and IL2RG, gifts from Jianwei Wang's lab, Institute of pathogen biology, Chinese Academy of Medical Sciences) were amplified from the constructs encoding the corresponding genes with introduction of G>A mutations through mutagenesis PCR. The amplified products were cloned into the pLenti-CMV-MCS-mCherry backbone through Gibson cloning method.

### Mammalian Cell Lines and Cell Culture

Mammalian cell lines were cultured Dulbecco's Modified Eagle Medium (10-013-CV, Corning, Tewksbury, MA, USA), adding 10% fetal bovine serum (Lanzhou Bailing Biotechnology Co., Ltd., Lanzhou, China), supplemented with 1% penicillin-streptomycin under 5% CO₂ at 37° C. The Adar1-KO cell line was purchased from EdiGene China, and the genotyping results were also provided by EdiGene China.

The HeLa and B16 cell lines were from Z. Jiang's laboratory (Peking University). And the HEK293T cell line was from C. Zhang's laboratory (Peking University). RD cell line was from J Wang's laboratory (Institute of Pathogen Biology, Peking Union Medical College & Chinese Academy of Medical Sciences). SF268 cell lines were from Cell Center, Institute of Basic Medical Sciences, Chinese Academy of Medical Sciences. A549 and SW13 cell lines were from EdiGene Inc. HepG2, HT29, NIH3T3, and MEF cell lines were maintained in our laboratory at Peking University. These mammalian cell lines were cultured in Dulbecco's Modified Eagle Medium (Corning, 10-013-CV) with 10% fetal bovine serum (CellMax, SA201.02), additionally supplemented with 1% penicillin-streptomycin under 5% CO₂ at 37° C. Unless otherwise described, cells were transfected with the X-tremeGENE HP DNA transfection reagent (Roche, 06366546001) according to the manufacturer's instruction.

The human primary pulmonary fibroblasts (#3300) and human primary bronchial epithelial cells (#3210) were purchased from ScienCell Research Laboratories, Inc. and were cultured in Fibroblast Medium (ScienCell, #2301) and Bronchial Epithelial Cell Medium (ScienCell, #3211), respectively. Both media were supplemented with 15% fetal bovine serum (BI) and 1% penicillin-streptomycin. The primary GM06214 and GM01323 cells were ordered from Coriell Institute for Medical Research and cultured in Dulbecco's Modified Eagle Medium (Corning, 10-013-CV) with 15% fetal bovine serum (BI) and 1% penicillin-streptomycin. All cells were cultured under 5% CO₂ at 37° C.

### Reporter System Transfection, FACS Analysis and Sanger Sequencing

For dual fluorescence reporter editing experiments, 293T-WT cells or 293T-Adar1-KO cells were seeded in 6 wells plates (6×10⁵ cells/well), 24 hours later, 1.5 μg reporter plasmids and 1.5 μg dRNA plasmids were co-transfected using the X-tremeGENE HP DNA transfection reagent (06366546001; Roche, Mannheim, German), according to the supplier's protocols. 48 to 72 hours later, collected cells and performed FACS analysis. For further confirming the reporter mRNA editing, we sorted the EGFP-positive cells from 293T-WT cells transfected with reporter and dRNA plasmids using a FACS Aria flow cytometer (BD Biosciences), followed by total RNA isolation (TIANGEN, DP430). Then the RNA was reverse-transcribed into cDNA via RT-PCR (TIANGEN, KR103-04), and the targeted locus were PCR amplified with the corresponding primer pairs (23 PCR cycles) and the PCR products were purified for Sanger sequencing.

For Adar1(p110), Adar1(p150) or Adar2 rescue and overexpression experiments, 293T-WT cells or 293T-Adar1-KO cells were seeded in 12 wells plates (2.5×10⁵ cells/well), 24 hours later, 0.5 μg reporter plasmids, 0.5 μg dRNA plasmids and 0.5 μg Adar1/2 plasmids (pLenti backbone as control) were co-transfected using the X-tremeGENE HP DNA transfection reagent (06366546001, Roche, Mannheim, German). 48 to 72 hours later, collected cells and performed FACS analysis.

For endogenous mRNA experiments, 293T-WT cells were seeded in 6 wells plates (6×10⁵ cells/well), When approximately 70% confluent, 3 μg dRNA plasmids were transfected using the X-tremeGENE HP DNA transfection reagent (06366546001, Roche, Mannheim, German). 72 hours later, collected cells and sorted GFP-positive or BFP-positive cells (according to the corresponding fluorescence maker) via FACS for the following RNA isolation.

### Isolation and Culture of Human Primary T Cells

Primary human T cells were isolated from leukapheresis products from healthy human donor. Briefly, Peripheral blood mononuclear cells (PBMCs) were isolated by Ficoll centrifugation (Dakewei, AS1114546), and T cells were isolated by magnetic negative selection using an EasySep Human T Cell Isolation Kit (STEMCELL, 17951) from PBMCs. After isolation, T cells were cultured in X-vivo15 medium, 10% FBS and IL2 (1000 U/ml) and stimulated with CD3/CD28 DynaBeads (ThermoFisher, 11131D) for 2 days. Leukapheresis products from healthy donors were acquired from AllCells LLC China. All healthy donors provided informed consent.

### Lenti-Virus Package and Reporter Cells Line Construction

The expression plasmid was co-transfected into HEK293T-WT cells, together with two viral packaging plasmids, pR8.74 and pVSVG (Addgene) via the X-tremeGENE HP DNA transfection reagent. 72 hours later, the supernatant virus was collected and stored at −80° C. The HEK293T-WT cells were infected with lenti-virus, 72 hours later, mCherry-positive cells were sorted via FACS and cultured to select a single clone cell lines stably expressing dual fluorescence reporter system with much low EGFP background by limiting dilution method.

For the stable reporter cell lines, the reporter constructs (pLenti-CMV-MCS-PURO backbone) were co-transfected into HEK293T cells, together with two viral packaging plasmids, pR8.74 and pVSVG. 72 hours later, the supernatant virus was collected and stored at −80° C. The HEK293T cells were infected with lentivirus, then mCherry-positive cells were sorted via FACS and cultured to select a single clone cell lines stably expressing dual fluorescence reporter system without detectable EGFP background. The HEK293T ADAR1^{-/-} and TP53^{-/-} cell lines were generated according to a previously reported method⁶⁰. ADAR1-targeting sgRNA and PCR amplified donor DNA containing CMV-driven puromycin resistant gene were co-transfected into HEK293T cells. Then cells were treated with puromycin 7 days after transfection. Single clones were isolated from puromycin resistant cells followed by verification through sequencing and Western blot.

### RNA Editing of Endogenous or Exogenous-Expressed Transcripts

For assessing RNA editing on the dual fluorescence reporter, HEK293T cells or HEK293T ADAR1^{-/-} cells were seeded in 6-well plates (6×10⁵ cells/well). 24 hours later, cells were co-transfected with 1.5 μg reporter plasmids and 1.5 μg arRNA plasmids. To examine the effect of ADAR1^{p110}, ADAR1^{p150} or ADAR2 protein expression, the editing efficiency was assayed by EGFP positive ratio and deep sequencing.

HEK293T ADAR1^{-/-} cells were seeded in 12-well plates (2.5×10⁵ cells/well). 24 hours later, cells were co-transfected with 0.5 μg of reporter plasmids, 0.5 μg arRNA plasmids and 0.5 μg ADAR1/2 plasmids (pLenti backbone as control). The editing efficiency was assayed by EGFP positive ratio and deep sequencing.

To assess RNA editing on endogenous mRNA transcripts, HEK293T cells were seeded in 6-well plates (6×10⁵ cells/well). Twenty-four hours later, cells were transfected with 3 μg of arRNA plasmids. The editing efficiency was assayed by deep sequencing.

To assess RNA editing efficiency in multiple cell lines, 8-9×104 (RD, SF268, HeLa) or 1.5×10⁵ (HEK293T) cells were seeded in 12-well plates. For cells difficult to transfect, such as HT29, A549, HepG2, SW13, NIH3T3, MEF and B16, 2-2.5×10⁵ cells were seeded in 6-well plate. Twenty-four hours later, reporters and arRNAs plasmid were co-transfected into these cells. The editing efficiency was assayed by EGFP positive ratio.

To evaluate EGFP positive ratio, at 48 to 72 hrs post transfection, cells were sorted and collected by Fluorescence-activated cell sorting (FACS) analysis. The mCherry signal was served as a fluorescent selection marker for the reporter/arRNA-expressing cells, and the percentages of EGFP⁺/mCherry⁺ cells were calculated as the readout for editing efficiency.

For NGS quantification of the A to I editing rate, at 48 to 72 hr post transfection, cells were sorted and collected by FACS assay and were then subjected to RNA isolation (TIANGEN, DP420). Then, the total RNAs were reverse-transcribed into cDNA via RT-PCR (TIANGEN, KR103-04), and the targeted locus was PCR amplified with the corresponding primers listed in the following table.

| Name of Primer | Sequence (5′--->3′) |
|---|---|
| mCherry-SpeI- | TATAACTAGTATGGTGAGCAAGGGCGAGGAG |
| F | (SEQ ID NO: 12) |
| mCherry-BsmBI- | TATACGTCTCATCTACAGATTCTTCCGGCGT |
| R1 | GTATACCTTC |
| | (SEQ ID NO: 13) |
| EGFP-BsmBI-F1 | TATACGTCTCATAGAGATCCCCGGTCGCCAC |
| | CGTGAGCAAGGGCGAGGAGCTG |
| | (SEQ ID NO: 14) |

The PCR products were purified for Sanger sequencing or NGS (Illumina HiSeq X Ten).

### Testing in Multiple Cell Lines

Besides HEK293T (positive control) and HEK293T ADAR1^{-/-} (negative control) cells, one mouse cell line (NIH3T3) as well as seven human cell lines (RD, HeLa, SF268, A549, HepG2, HT-29, SW13) originating from different tissues and organs were selected to perform the experiment. For the cell lines with higher transfection efficiency, about 8-9×10⁴ cells (RD, HeLa, SF268) or 1.5×10⁵ (HEK293T) were plated onto each well of 12-well plate, as for the ones (A549, HepG2, HT-29, SW13, NIH3T3) which are difficult to transfect, 2-2.5×10⁵ cells were plated in 6-well plate. And all these cells were maintained in Dulbecco's modified Eagle's medium (DMEM, Corning) supplemented with 10% fetal bovine serum (FBS, CellMax) with 5% CO₂ in 37° C. 24 hrs later, CG2 reporter and 71 nt dRNA (35-C-35) plasmid were co-transfected into different type of cells with X-tremeGENE HP DNA transfection reagent (Roche). 48 hrs after transfection, cells were trypsinized and analyzed through FACS (BD). Because the cells with low transfection efficiency had quite fewer mCherry and BFP positive cells, we increased the total cell number for FACS analysis to 1×10⁵ for those cells plated onto 6-well plate.

### Example 1. Optimizing LEAPER by Using a CMV Promoter to Drive arRNA Expression

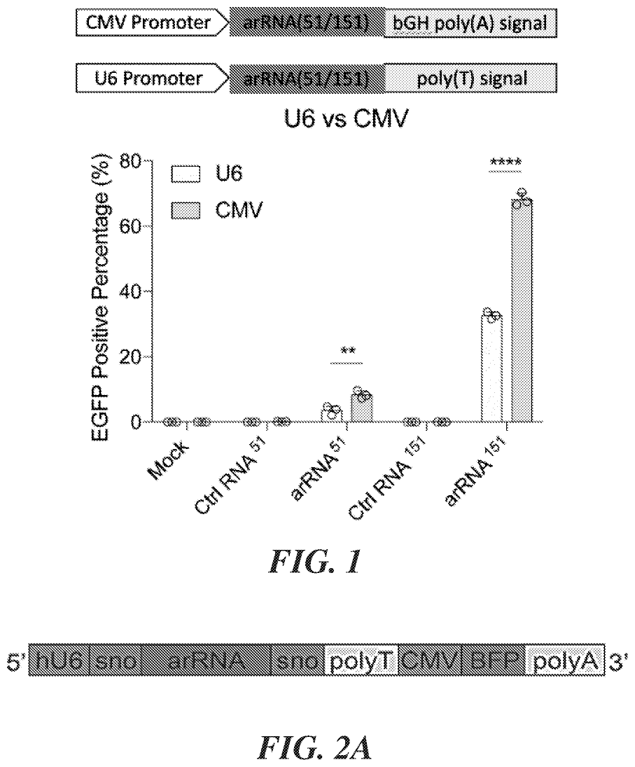

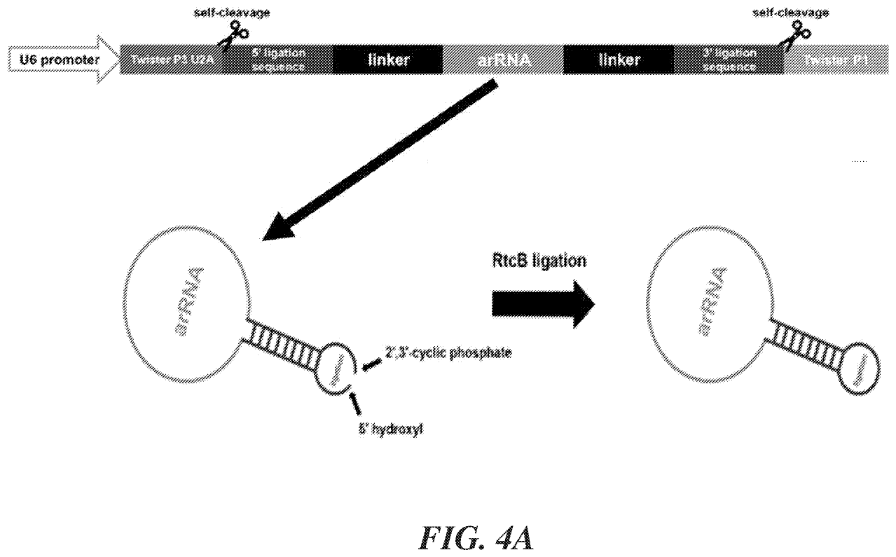

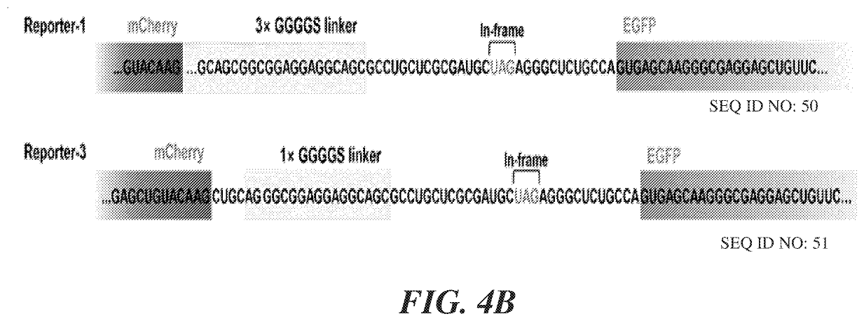

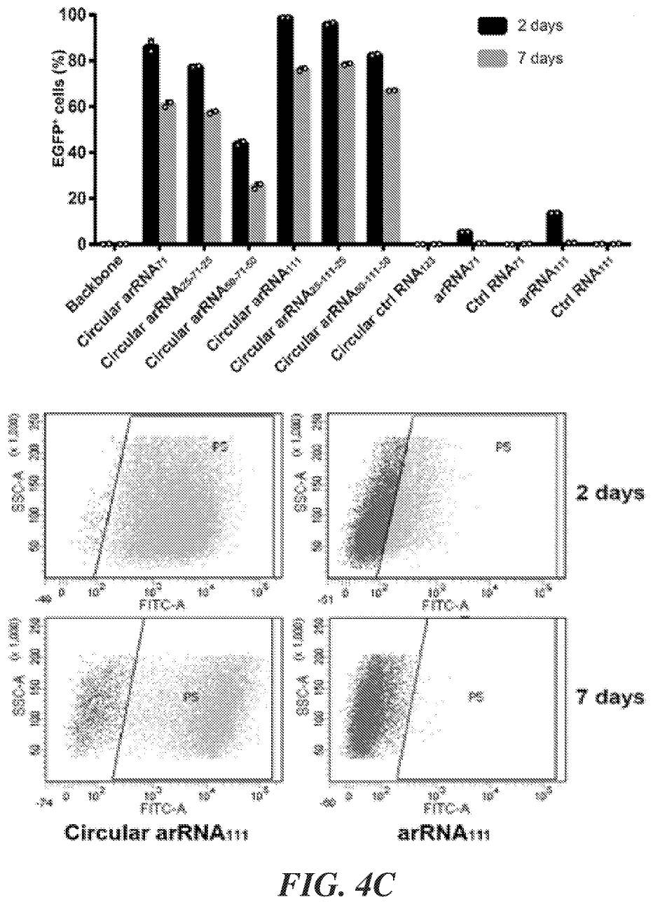

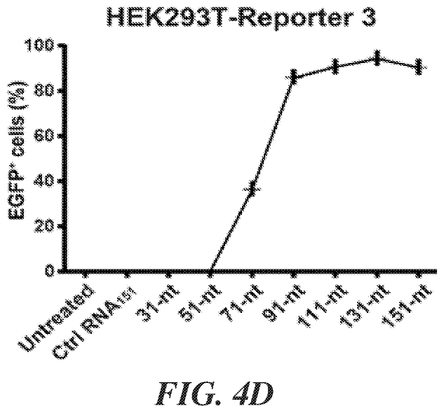

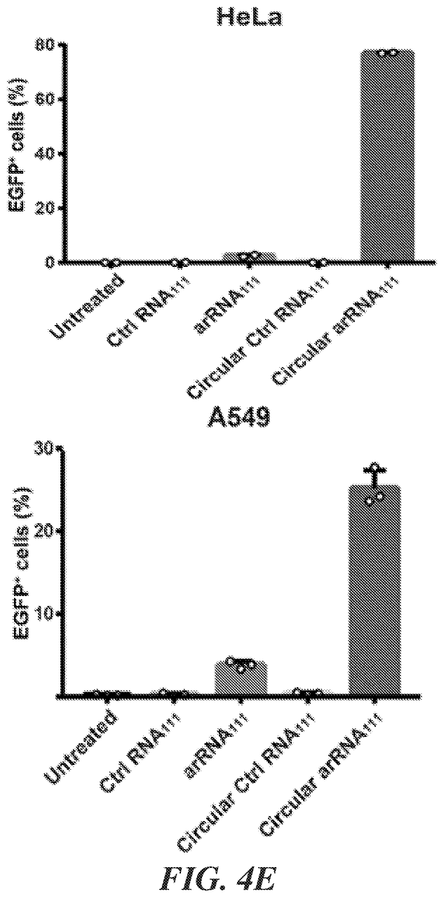

To test whether an RNA Polymerase II (Pol II) can improve editing efficiency, a plasmid expressing arRNA driven by a Pol II promoter (CMV) was constructed. Using a reporter system based on EGFP expression (Reporter 1, FIG. 4B), the RNA editing efficiency between arRNAs driven by CMV and U6 promoters was compared (arRNA₅₁). Untreated cells were used as a mock control (Mock). Cells transfected with a non-targeting RNA were also used as a control (Ctrl RNAs). It was found that CMV-arRNA outperforms U6-arRNA in RNA editing (FIG. 1).

| Name of Primer | Sequence (5′--->3′) |
|---|---|
| Ctrl RNA51 | UAAACCGAGGGAUCAUAGGGGACUGAAUC |
| | CACCAUUCUUCUCCCAAUCCCU |
| | (SEQ ID NO: 15) |
| Ctrl RNA151 | ACUACAGUUGCUCCGAUAUUUAGGCUACG |
| | UCAAUAGGCACUAACUUAUUGGCGCUGGU |
| | GAACGGACUUCCUCUCGAGUACCAGAAGA |
| | UGACUACAAAACUCCUUUCCAUUGCGAGU |
| | AUCGGAGUCUGGCUCAGUUUGGCCAGGGA |
| | GGCACU (SEQ ID NO: 16) |
| arRNA₅₁ | GCCCTTGCTCACTGGCAGAGCCCTCCAGC |
| | ATCGCGAGCAGGCGCTGCCTCC |
| | (SEQ ID NO: 17) |
| arRNA₁₅₁ | ACUACAGUUGCUCCGAUAUUUAGGCUACG |
| | UCAAUAGGCACUAACUUAUUGGCGCUGGU |
| | GAACGGACUUCCUCUCGAGUACCAGAAGA |
| | UGACUACAAAACUCCUUUCCAUUGCGAGU |
| | AUCGGAGUCUGGCUCAGUUUGGCCAGGGA |
| | GGCACU (SEQ ID NO: 18) |

### Example 2. RNA Editing Mediated by Sno-arRNA

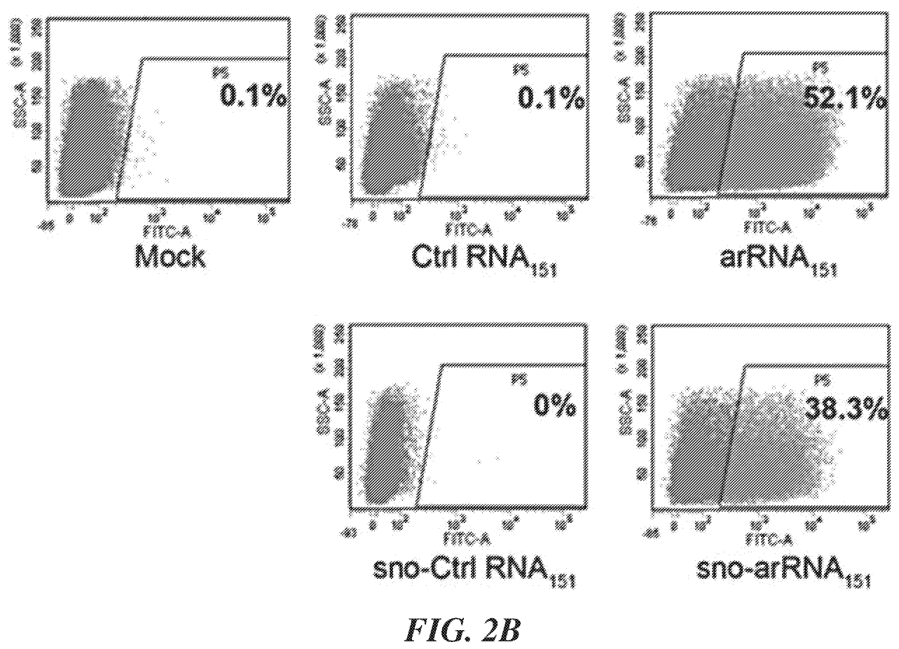

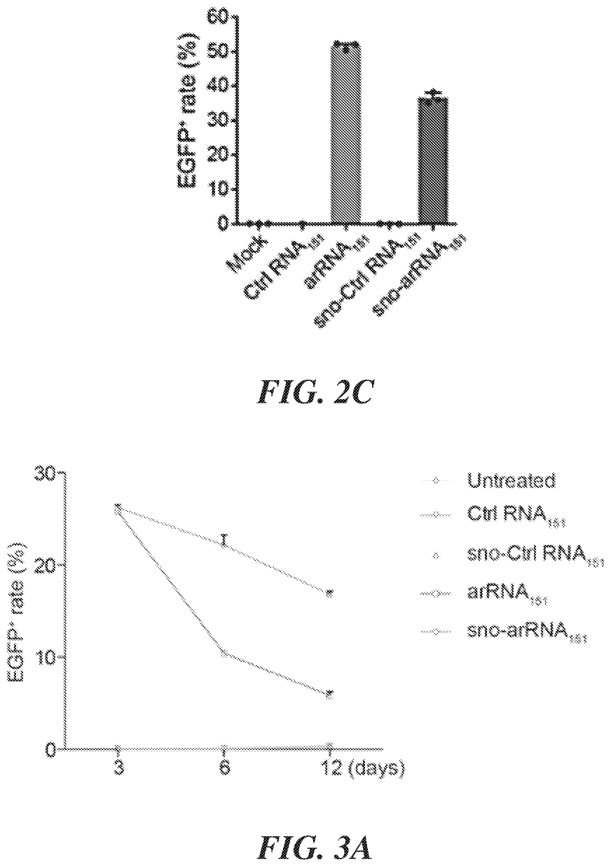

In order to stabilize arRNAs and enhance its nuclear localization, arRNA was engineered to have snoRNA ends. The 151-nt arRNA targeting fluorescence Reporter-1 was flanked with snoRNA ends (FIG. 2A):

5′ end:
(SEQ ID NO: 19)
GAGTGAGATCTTGGACCAATGATGACTTCCATACA
TGCATTCCTTGGAAAGCTGAACAAAATGAGTGGGA
ACTCTGTACTATCATCTTAGTTGAACTGAGGTCCA
CCGGGGGCTAA;
3′ end:
(SEQ ID NO: 20)
AAGATTGTGTGTGGATCGATGATGACTTCCATATA
TACATTCCTTGGAAAGCTGAACAAAATGAGTGAAA
ACTCTATACCGTCATTCTCGTCGAACTGAGGTCCA
GCACATTACTCCAACAG,
named sno-arRNA151.

Dual fluorescence reporter-1 comprises sequence of mCherry (SEQ ID NO: 21), sequence comprising 3×GS linker and the targeted A (SEQ ID NO: 22), and sequence of eGFP (SEQ ID NO: 23).

(SEQ ID NO: 21)
ATGGTGAGCAAGGGCGAGGAGGATAACATGGCCATCATCAAGGAG
TTCATGCGCTTCAAGGTGCACATGGAGGGCTCCGTGAACGGCCAC
GAGTTCGAGATCGAGGGCGAGGGCGAGGGCCGCCCCTACGAGGGC
ACCCAGACCGCCAAGCTGAAGGTGACCAAGGGTGGCCCCCTGCCC
TTCGCCTGGGACATCCTGTCCCCTCAGTTCATGTACGGCTCCAAG
GCCTACGTGAAGCACCCCGCCGACATCCCCGACTACTTGAAGCTG
TCCTTCCCCGAGGGCTTCAAGTGGGAGCGCGTGATGAACTTCGAG
GACGGCGGCGTGGTGACCGTGACCCAGGACTCCTCCCTGCAGGAC
GGCGAGTTCATCTACAAGGTGAAGCTGCGCGGCACCAACTTCCCC
TCCGACGGCCCCGTAATGCAGAAGAAGACCATGGGCTGGGAGGCC
TCCTCCGAGCGGATGTACCCCGAGGACGGCGCCCTGAAGGGCGAG
ATCAAGCAGAGGCTGAAGCTGAAGGACGGCGGCCACTACGACGCT
GAGGTCAAGACCACCTACAAGGCCAAGAAGCCCGTGCAGCTGCCC
GGCGCCTACAACGTCAACATCAAGTTGGACATCACCTCCCACAAC
GAGGACTACACCATCGTGGAACAGTACGAACGCGCCGAGGGCCGC
CACTCCACCGGCGGCATGGACGAGCTGTACAAG
(sequence of mCherry)
(SEQ ID NO: 22)
CTGCAGGGCGGAGGAGGCAGCGGCGGAGGAGGCA
GCGGCGGAGGAGGCAGCAGAAGGTATACACGCC
GGAAGAATCTGT [symbol] GAGATCCCCGGTCGCCACC
(sequence comprising 3xGS linker
(shown as italic and
bold characters) and the targeted A (shown as
larger and bold A))
(SEQ ID NO: 23)
GTGAGCAAGGGCGAGGAGCTGTTCACCGGGGTGGTGCCCATCCTG
GTCGAGCTGGACGGCGACGTAAACGGCCACAAGTTCAGCGTGTCC
GGCGAGGGCGAGGGCGATGCCACCTACGGCAAGCTGACCCTGAAG
TTCATCTGCACCACCGGCAAGCTGCCCGTGCCCTGGCCCACCCTC
GTGACCACCCTGACCTACGGCGTGCAGTGCTTCAGCCGCTACCCC
GACCACATGAAGCAGCACGACTTCTTCAAGTCCGCCATGCCCGAA
GGCTACGTCCAGGAGCGCACCATCTTCTTCAAGGACGACGGCAAC
TACAAGACCCGCGCCGAGGTGAAGTTCGAGGGCGACACCCTGGTG
AACCGCATCGAGCTGAAGGGCATCGACTTCAAGGAGGACGGCAAC
ATCCTGGGGCACAAGCTGGAGTACAACTACAACAGCCACAACGTC
TATATCATGGCCGACAAGCAGAAGAACGGCATCAAGGTGAACTTC
AAGATCCGCCACAACATCGAGGACGGCAGCGTGCAGCTCGCCGAC
CACTACCAGCAGAACACCCCCATCGGCGACGGCCCCGTGCTGCTG
CCCGACAACCACTACCTGAGCACCCAGTCCGCCCTGAGCAAAGAC
CCCAACGAGAAGCGCGATCACATGGTCCTGCTGGAGTTCGTGACC
GCCGCCGGGATCACTCTCGGCATGGACGAGCTGTACAAGTAA.
(sequence of eGFP)

Then the human U6-derived sno-arRNA151, arRNA151, sno-Ctrl RNA151 or Ctrl RNA151 was transfected into HEK293T cells along with Reporter-expressing plasmids. Forty-eight hours post transfection, the EGFP positive rate was quantified via FACS analysis. The FACS results showed sno-arRNA151 flanked by snoRNA ends could mediated targeted RNA editing on Reporter mRNA with almost 38% EGFP positive rate, lower than that of the linear arRNAs (FIGS. 2B-2C).

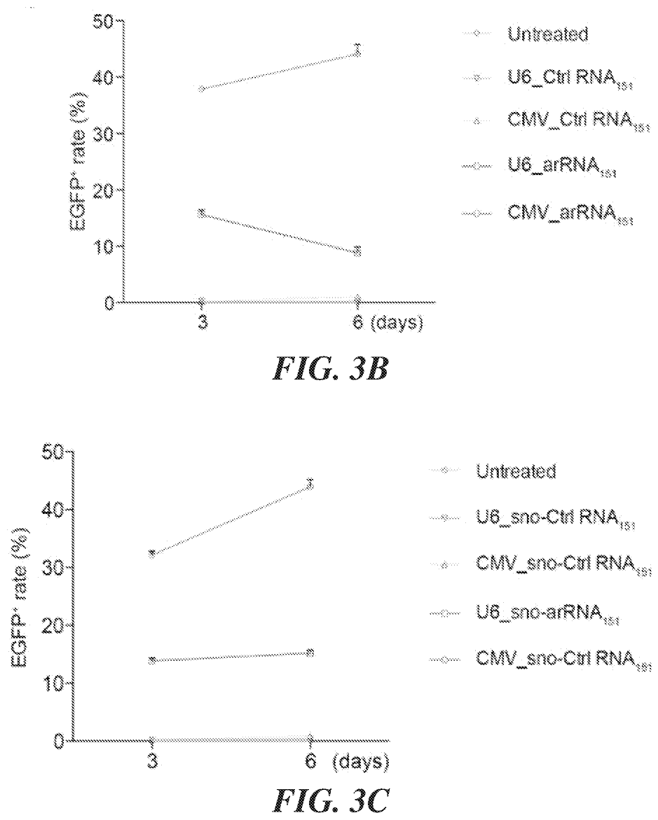

To test if snoRNA ends stabilize the arRNA, the EGPF positive rate was measured at different time points. The sno-arRNA₁₅₁, arRNA₁₅₁, sno-Ctrl RNA₁₅₁ or Ctrl RNA₁₅₁ under human U6 promoter were transfected into HEK293T-Reporter cells. The EGFP positive rate was measured at 3 times points: 3 days, 6 days and 12 days post transfection. The results showed that sno-arRNA exhibited higher editing efficiency than arRNA during the extended time period, indicating that snoRNA ends could protect arRNA from degradation and enhance the abundance of sno-arRNA (FIG. 3A).

To further enhance the expression level of arRNA, a virus-based strong promoter-CMV was used to express arRNA or sno-arRNA. The results showed that CMV-promoter expressed arRNA (CMV_arRNA) exhibited much higher editing efficiency than U6-promoter expressed arRNA (U6_arRNA). Besides, the EGFP positive rate of U6_arRNA group declined with prolonged time, while the EGFP positive rate of CMV_arRNA still increased at 6 days post transfection. Similarly, CMV-promoter expressed sno-arRNA (CMV_sno-arRNA) achieved much higher editing efficiency than U6-promoter expressed sno-arRNA (U6_sno-arRNA) (FIG. 3B and FIG. 3C).

Based on the above results, it was demonstrated that snoRNA ends could stabilize arRNA, and arRNA or sno-arRNA driven by CMV promoter could increase its expression level. Both of these two strategy could significantly boost the editing efficiency of arRNA.

### Example 3. RNA Editing Mediated by Circular arRNA

HEK293T cells stably expressing the Reporter-1 containing an in-frame stop codon between mCherry and EGFP (FIG. 4B) were transfected with plasmids expressing circular arRNA₇₁ and circular arRNA₁₁₁ both targeting the Reporter-1. The EGFP fluorescence indicates the efficiency of target editing on RNA. To test most efficient arRNA architecture, circular arRNAs were flanked with 25-nt or 50-nt linker connecting to both ends ligation sequence (FIG. 4A). For comparison, linear (non-circular) arRNA₇₁ and arRNA₁₁₁ were also transfected at the same time. It turned out that circular arRNAs strongly improved both the ratios of EGFP⁺ cells and EGFP intensity (FIG. 4C) compared with linear arRNAs, while circular arRNA with 25-nt or 50-nt linker weakened the improvement of efficiency (FIG. 4C).

In HEK293T stably expressing the Reporter-3 (FIG. 4B), the effect of length on editing efficiency was further tested. Based on the reporter EGFP ratios, the length of circular arRNA correlated with the editing efficiency positively, peaking at 111- to 151-nt (FIG. 4D) and 71-nt was the minimal length for circular arRNA activity (FIG. 4D).

Dual fluorescence reporter-3 comprises sequence of mCherry (SEQ ID NO:21), sequence comprising 1×GS linker (shown as italic and bold characters) and the targeted A (SEQ ID NO:24), and sequence of eGFP (SEQ ID NO:23).

(SEQ ID NO: 24)
CTGCAGGGCGGAGGAGGCAGCGCCTGCTCGCGATGCT [symbol]
GAGGGCTCTGCCA
(sequence comprising 1xGS linker
(shown as italic and bold characters)
and the targeted A
(shown as larger and bold A))

The circular arRNAs were used in HeLa and A549 cells in which LEAPER efficiencies are relatively low among multiple cell lines we have tested because of low expression level of ADAR1 or high expression of ADAR3. It turned out that circular arRNAs significantly boosted the editing efficiency in both cell lines based on EGFP reporter assays (FIG. 4E).

| Name | Sequence (5′ ---> 3′) |
|---|---|
| 5′ ligation | AACCAUGCCGACUGAUGGCAG (SEQ ID NO: 25) |
| sequence | |
| 3′ ligation | CUGCCAUCAGUCGGCGUGGACUGUAG (SEQ ID NO: 26) |
| sequence | |
| Circular arRNA₇₁ | ACAGCUCCUCGCCCUUGCUCACUGGCAGAGCCCUCCAGCAUCGCGA |
| | GCAGGCGCUGCCUCCUCCGCCGCUG (SEQ ID NO: 27) |
| Circular | CACACACAAACACACACAACACAACACAGCUCCUCGCCCUUGCUCA |
| arRNA₂₅₋₇₁₋₂₅ | CUGGCAGAGCCCUCCAGCAUCGCGAGCAGGCGCUGCCUCCUCCGCC |
| | GCUGCAACACACAAAACCACCACACCAAC (SEQ ID NO: 28) |
| Circular | ACCACACACACAACCACCACACACACACACACAAACACACACAACA |
| arRNA₅₀₋₇₁₋₅₀ | CAACACAGCUCCUCGCCCUUGCUCACUGGCAGAGCCCUCCAGCAUC |
| | GCGAGCAGGCGCUGCCUCCUCCGCCGCUGCAACACACAAAACCACC |
| | ACACCAACCCCAACAACCACACACCCACACAAC (SEQ ID NO: 29) |
| Circular | GAUGGGCACCACCCCGGUGAACAGCUCCUCGCCCUUGCUCACUGG |
| arRNA₁₁₁ | CAGAGCCCUCCAGCAUCGCGAGCAGGCGCUGCCUCCUCCGCCGCUG |
| | CCUCCUCCGCCGCUGCCUCC (SEQ ID NO: 30) |
| Circular | CACACACAAACACACACAACACAACGAUGGGCACCACCCCGGUGA |
| arRNA₂₅₋₁₁₁₋₂₅ | ACAGCUCCUCGCCCUUGCUCACUGGCAGAGCCCUCCAGCAUCGCGA |
| | GCAGGCGCUGCCUCCUCCGCCGCUGCCUCCUCCGCCGCUGCCUCCC |
| | AACACACAAAACCACCACACCAAC (SEQ ID NO: 31) |
| Circular | ACCACACACACAACCACCACACACACACACACAAACACACACAACA |
| arRNA₅₀₋₁₁₁₋₅₀ | CAACGAUGGGCACCACCCCGGUGAACAGCUCCUCGCCCUUGCUCAC |
| | UGGCAGAGCCCUCCAGCAUCGCGAGCAGGCGCUGCCUCCUCCGCCG |
| | CUGCCUCCUCCGCCGCUGCCUCCCAACACACAAAACCACCACACCA |
| | ACCCCAACAACCACACACCCACACAAC (SEQ ID NO: 32) |
| Circular ctrl | UUGCCAUGUGUAUGUGGGGAGACGGUCGGGUCCAGAUAUUCGUAU |
| RNA123 | CUGUCGAGUAGAGUGUGGGCUCCCCACAUACUCUGAUGAUCCAGA |
| | GACGAUAUUACGUCUCAGGAUCAUUCAUGGCAA (SEQ ID NO: 33) |
| arRNA₇₁ | ACAGCUCCUCGCCCUUGCUCACUGGCAGAGCCCUCCAGCAUCGCGA |
| | GCAGGCGCUGCCUCCUCCGCCGCUG (SEQ ID NO: 34) |
| Ctrl arRNA₇₁ | UUUCAGCUAUACCUGCCCGGUAUAAAGGGACGUUCACACCGCGAU |
| | GUUCUCUGCUGGGGAAUUGCGCGAUA (SEQ ID NO: 35) |
| arRNA₁₁₁ | GAUGGGCACCACCCCGGUGAACAGCUCCUCGCCCUUGCUCACUGG |
| | CAGAGCCCUCCAGCAUCGCGAGCAGGCGCUGCCUCCUCCGCCGCUG |
| | CCUCCUCCGCCGCUGCCUCC (SEQ ID NO: 36) |
| Ctrl arRNA₁₁₁ | UACCGCUACAGCCACGCUGAUUUCAGCUAUACCUGCCCGGUAUAA |
| | AGGGACGUUCACACCGCGAUGUUCUCUGCUGGGGAAUUGCGCGAU |
| | AUUCAGGAUUAAAAGAAGUGC(SEQ ID NO: 37) |
| Ctrl arRNA₁₅₁ | ACUACAGUUGCUCCGAUAUUUAGGCUACGUCAAUAGGCACUAACU |
| (circular) | UAUUGGCGCUGGUGAACGGACUUCCUCUCGAGUACCAGAAGAUGA |
| | CUACAAAACUCCUUUCCAUUGCGAGUAUCGGAGUCUGGCUCAGUU |
| | UGGCCAGGGAGGCACU (SEQ ID NO: 38) |
| 31-nt | ACUGGCAGAGCCCUCCAGCAUCGCGAGCAGG (SEQ ID NO: 39) |
| 51-nt | GCCCUUGCUCACUGGCAGAGCCCUCCAGCAUCGCGAGCAGGCGCU |
| | GCCUCC (SEQ ID NO: 40) |
| 71-nt | ACAGCUCCUCGCCCUUGCUCACUGGCAGAGCCCUCCAGCAUCGCGA |
| | GCAGGCGCUGCCUCCUCCGCCCUGC (SEQ ID NO: 41) |
| 91-nt | ACCCCGGUGAACAGCUCCUCGCCCUUGCUCACUGGCAGAGCCCUCC |
| | AGCAUCGCGAGCAGGCGCUGCCUCCUCCGCCCUGCAGCUUGUACA |
| | (SEQ ID NO: 42) |
| 111-nt | GAUGGGCACCACCCCGGUGAACAGCUCCUCGCCCUUGCUCACUGG |
| | CAGAGCCCUCCAGCAUCGCGAGCAGGCGCUGCCUCCUCCGCCCUGC |
| | AGCUUGUACAGCUCGUCCAU (SEQ ID NO: 43) |
| 131-nt | GCUCGACCAGGAUGGGCACCACCCCGGUGAACAGCUCCUCGCCCU |
| | UGCUCACUGGCAGAGCCCUCCAGCAUCGCGAGCAGGCGCUGCCUC |
| | CUCCGCCCUGCAGCUUGUACAGCUCGUCCAUGCCGCCGGUG (SEQ |
| | ID NO: 44) |
| 151-nt | UCGCCGUCCAGCUCGACCAGGAUGGGCACCACCCCGGUGAACAGC |
| | UCCUCGCCCUUGCUCACUGGCAGAGCCCUCCAGCAUCGCGAGCAG |
| | GCGCUGCCUCCUCCGCCCUGCAGCUUGUACAGCUCGUCCAUGCCGC |
| | CGGUGGAGUGGCGGC (SEQ ID NO: 45) |
| arRNA₁₁₁ | GAUGGGCACCACCCCGGUGAACAGCUCCUCGCCCUUGCUCACUGG |
| | CAGAGCCCUCCAGCAUCGCGAGCAGGCGCUGCCUCCUCCGCCGCUG |
| | CCUCCUCCGCCGCUGCCUCC (SEQ ID NO: 46) |
| Ctrl arRNA₁₁₁ | UACCGCUACAGCCACGCUGAUUUCAGCUAUACCUGCCCGGUAUAA |
| | AGGGACGUUCACACCGCGAUGUUCUCUGCUGGGGAAUUGCGCGAU |
| | AUUCAGGAUUAAAAGAAGUGC (SEQ ID NO: 47) |
| Circular | GAUGGGCACCACCCCGGUGAACAGCUCCUCGCCCUUGCUCACUGG |
| arRNA₁₁₁ | CAGAGCCCUCCAGCAUCGCGAGCAGGCGCUGCCUCCUCCGCCGCUG |
| | CCUCCUCCGCCGCUGCCUCC (SEQ ID NO: 48) |
| Circular Ctrl | UACCGCUACAGCCACGCUGAUUUCAGCUAUACCUGCCCGGUAUAA |
| arRNA₁₁₁ | AGGGACGUUCACACCGCGAUGUUCUCUGCUGGGGAAUUGCGCGAU |
| | AUUCAGGAUUAAAAGAAGUGC (SEQ ID NO: 49) |

### Example 4. Correcting the Premature Stop Codon Using the LEAPER System with Circular arRNAs

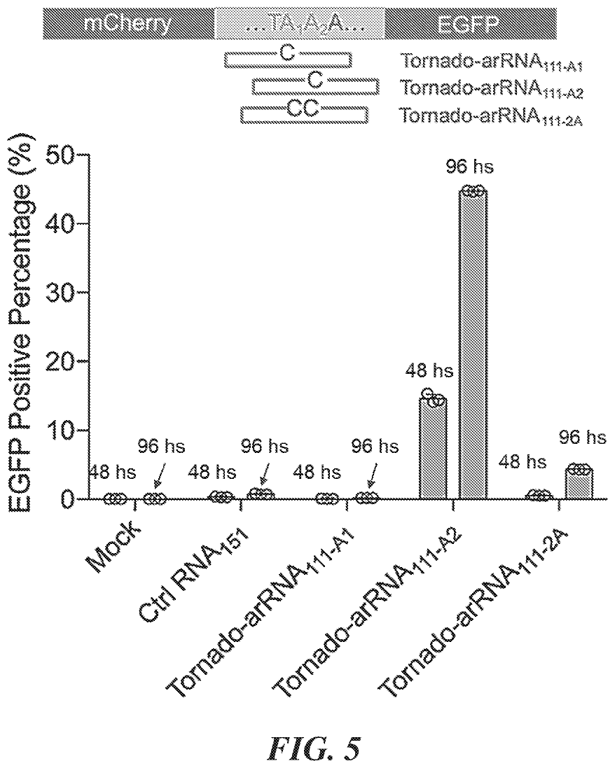

Many diseases are caused by premature stop codon (PTC). To examine the potential of LEAPER-circular arRNA in treating PTC-related disease, an in frame UAA stop codon reporter was constructed. Three versions of circular arRNAs (i.e., Tornado-arRN_{A111-A1}, Tornado-arRN_{A111-A2} and Tornado-arRN_{A111-2A}) were designed to target the UAA site of this reporter and compared the EGFP positive percentage of three different arRNAs. It turned out that that Tornado-arRNA_{111-A2}, which targets the second adenosine of the TAA codon, is more efficient (FIG. 5).

### Example 5. Modulation of mRNA Splicing Using the LEAPER System with Circular arRNAs

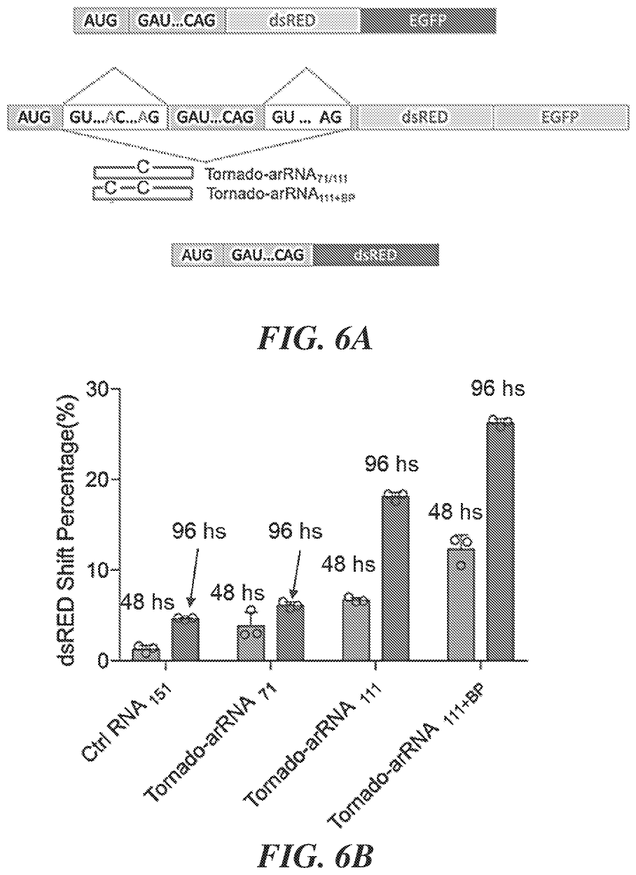

Manipulating mRNA splicing to generate novel splicing products is a strategy to cure diseases, such as Duchene muscular dystrophy. RG6 splicing reporter was used to test if LEAPER-circular arRNAs could change splicing acceptor site. Three versions of circular arRNAs were designed to target RG6 splicing acceptor site, including Tornado-arRNA₇₁ (71-nt), Tornado-arRNA₇₁(11-nt), and Tornado-arRNA_{71+P} (111-nt targeting both acceptor site and a branch point). In HEK293T cell, the RG6 reporter expressed more dsRNA protein over EGFP protein if the splicing if the splicing pattern was changed by LEAPER-Tornado (FIG. 6A). We found that LEAPER-circular arRNAs can efficiently target the cellular splicing machinery (FIG. 6B).

### Example 6. Lentiviral Delivery of the LEAPER System with Circular arRNAs

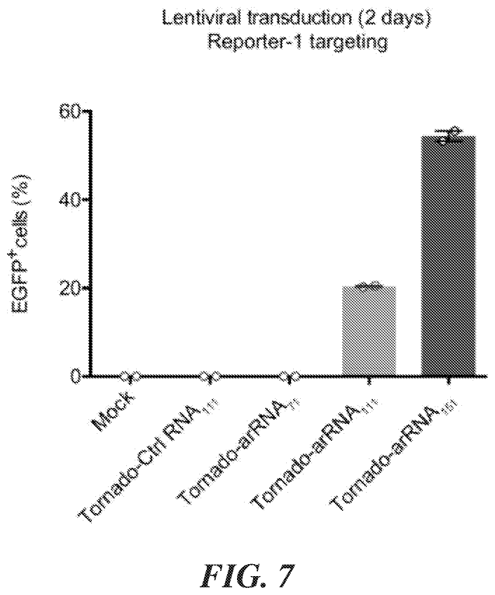

Continuous dosing of therapeutic RNA is important for its efficacy. It was tested whether lentiviral delivery of LEAPER with circular arRNAs could achieve RNA editing. It was found that LEAPER with circular arRNAs could work through lentivirus delivery, by which various Tornado-arRNA could integrate into genome and stably expressed. The length of Tornado-arRNA correlated positively with the editing efficiency (FIG. 7).

### Example 7. RNA Editing Mediated by Circular arRNAs Produced In Vitro

In addition to expression of circular arRNAs in vivo using the Tornado system, the arRNAs can alternatively be circularized in vitro.

As shown in FIGS. 8A-8B, the arRNA was generated in vitro using a T4 RNA ligase with or without a DNA splint (i.e., single-stranded DNA adaptor). Briefly, an in vitro RNA transcript corresponding to the arRNA was obtained using in vitro transcription (IVT) of a DNA template. Then, the IVT product was ligated a T4 RNA ligase (T4 Rnl1 or T4 Rnl2) bound to a DNA splint (FIG. 8A), or directly without a DNA splint (FIG. 8B). Both T4 Rnl1 and T4 Rnl2 could efficiently circularize linear ssRNAs in vitro.

FIG. 8C illustrates a method to produce circular arRNAs in vitro based on a Group I catalytic intron. A typical Group I catalytic intron comprises, from the 5′ end to the 3′ end: a 5′ exon comprising a 5′ exon sequence recognizable by a 5′ catalytic Group I intron fragment (Exon 1), 5′ catalytic Group I intron fragment, 3′ catalytic Group I intron fragment, and a 3′ exon comprising a 3′ exon sequence recognizable by the 3′ catalytic Group I intron fragment (Exon 2). A linear RNA construct with an arRNA sequence can be made to allow auto-catalysis of the Group I intron fragments in order to join the two ends of the arRNA sequence and obtain a circular arRNA after self-splicing by the Group I intron. The linear construct comprises, from 5′ to 3′, 3′ catalytic Group I intron fragment, a 3′ exon (Exon 2), an arRNA sequence, a 5′ exon (Exon 1), and 5′ Group I intron. The linear RNA construct was obtained using IVT. The purified IVT product was circularized by adding GTPs and Mg²⁺, which was incubated at 55° C. for 15 minutes. To remove the linear RNA precursor, the reaction mixture was treated with RNase R or purified using High Performance Liquid Chromatography (HPLC). After RNase R treatment and/or HPLC, the purified RNA was treated with alkaline phosphatase to remove the phosphate group if necessary.

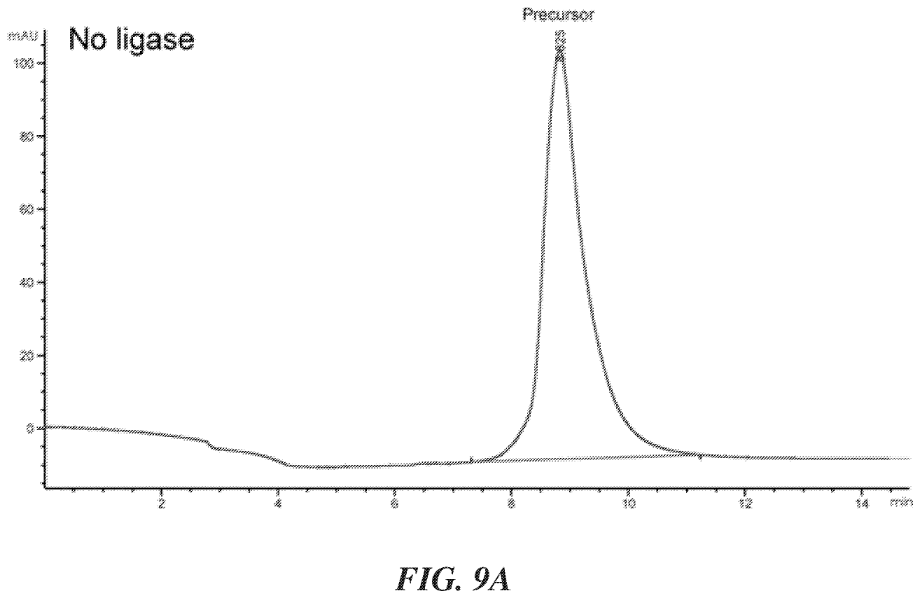

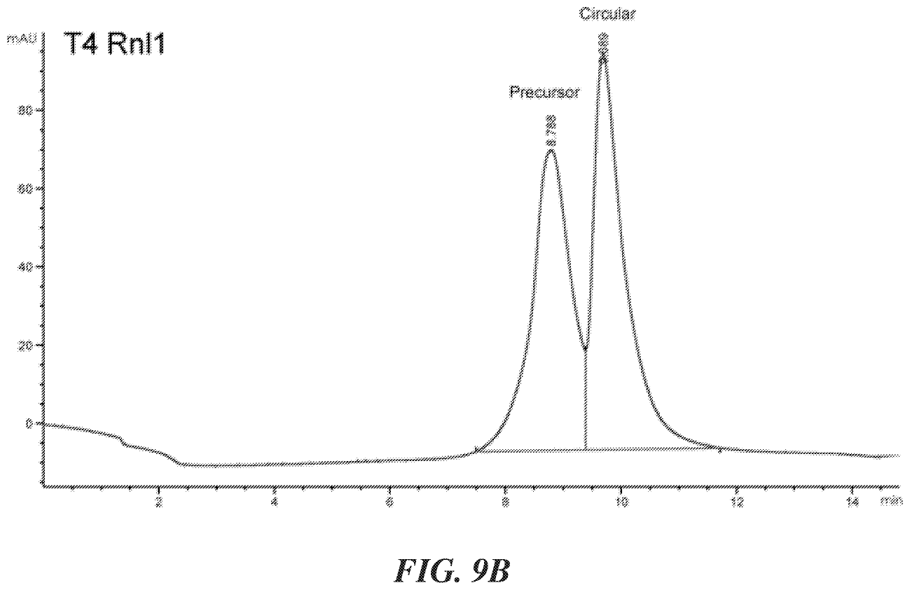

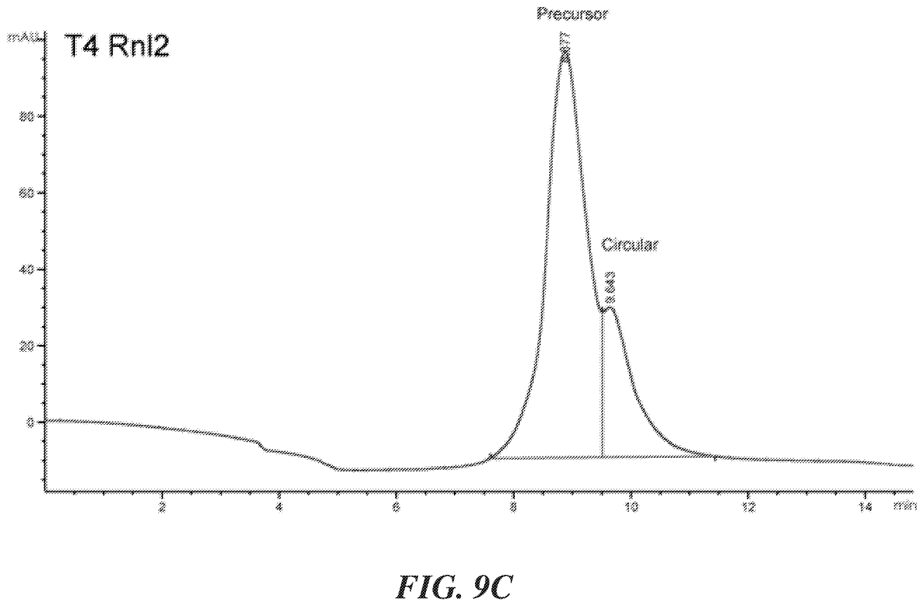

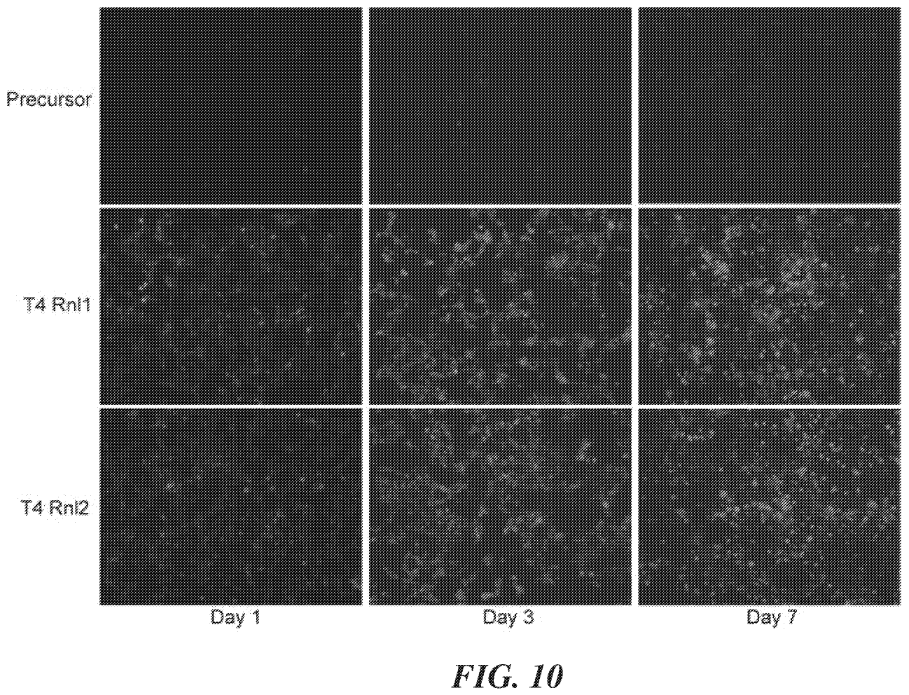

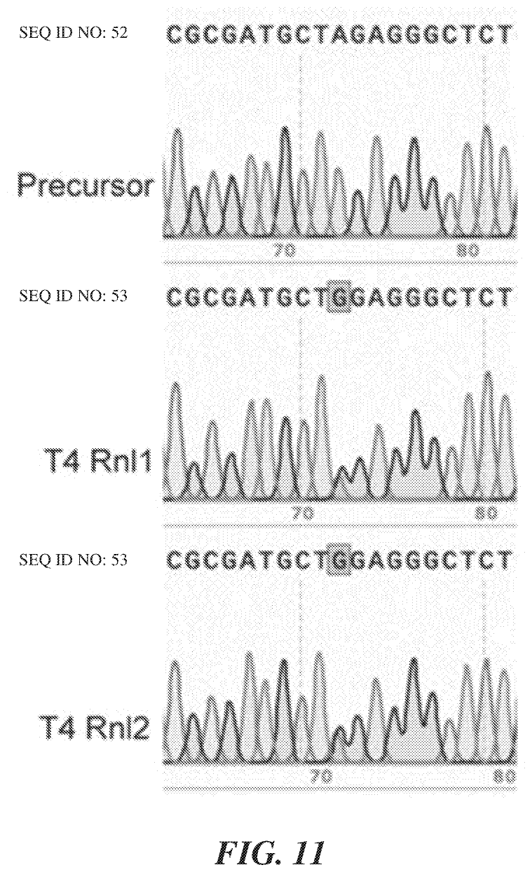

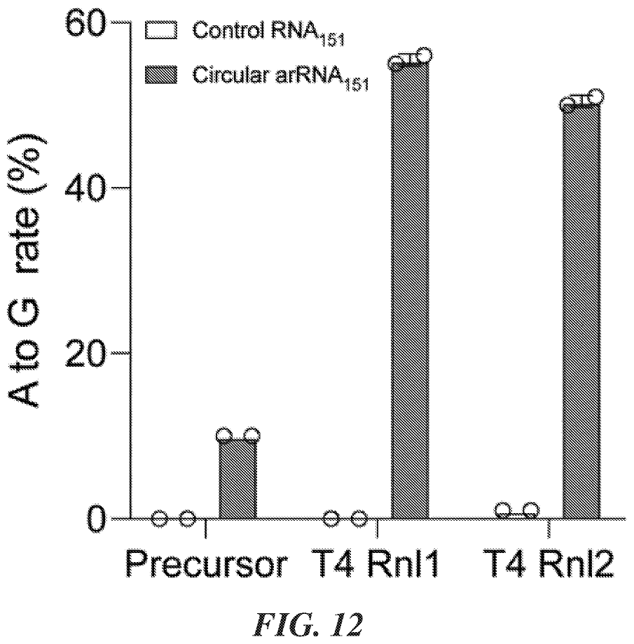

Using the above in vitro methods, we generated a circular arRNA targeting Reporter-1 as described in Example 2. As shown in FIGS. 9A-9C, the T4 Rnl1 efficiently circularized arRNA. The circularization efficiency was about 60% as determined by HPLC analysis. We transfected the purified circular arRNA into a Reporter-1 stable cell line. The arRNA efficient turn on the EGFP expression after transfection of the circular arRNA for 24 hours (FIG. 10). Almost all cells showed expression of EGFP in circular arRNA₁₅₁ treated group. Based on the Sanger sequencing results (FIG. 11), we determined that the editing efficiency was about 60% after transfection for 48 hours. This editing efficiency is consistent with the deep sequencing results (FIG. 12). We continued to culture the cells for half a month to test the duration of the impact by the circular arRNA. As shown in FIG. 10, the vast majority of cells transfected with circular arRNAs continued to express EGFP for at least one week post transfection.

Next, we used the in vitro methods to generate circular arRNAs targeting endogenous transcripts. The length of the arRNAs was 151 nt. We successfully utilized T4 Rnl-based and Group I catalytic intron-based methods to produce circular arRNAs targeting endogenous transcripts in vitro. The purified circular arRNAs were used to transfect HEK293T and mouse embryo fibroblasts (MEF).

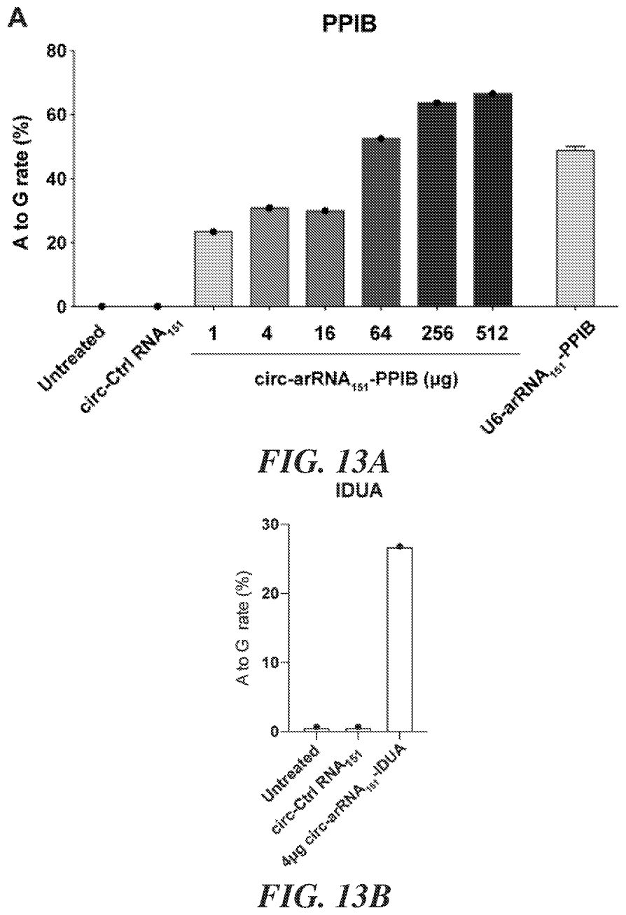

We targeted the UAG motifs in the transcripts of PPIB in HEK293T. The targeted adenosine sites in the PPIB transcripts were edited by their corresponding circular arRNAs. We then tested whether the editing efficiency was dose-dependent. The data in FIG. 13A showed that the editing efficiency was dose-dependent and a high dose of circular arRNA tended to yield higher editing rates.

We examined the potential of using a circular arRNA to treat a monogenic disease, Hurler syndrome. Hurler syndrome is the most severe subtype of Mucopolysaccharidosis type I (MPS I) due to deficiency of IDUA, a lysosomal metabolic enzyme responsible for the degradation of mucopolysaccharides. We chose MEF cells that were originally isolated from a mouse model of Hurler syndrome. These MEF cells contained a homozygous TGG-to-TAG mutation in exon 9 of the IDUA gene. We transfected a circular arRNA targeting IDUA transcripts into MEF and determined the editing efficiency. The deep sequence results in FIG. 13B show that the editing efficiency of the IDUA transcripts was about 27%.

### REFERENCES

1 Porteus, M. H. & Carroll, D. Gene targeting using zinc finger nucleases. Nat Biotechnol 23, 967-973 (2005).
2 Boch, J. et al. Breaking the code of DNA binding specificity of TAL-type III effectors. Science 326, 1509-1512 (2009).
3 Moscou, M. J. & Bogdanove, A. J. A simple cipher governs DNA recognition by TAL effectors. Science 326, 1501 (2009).
4 Miller, J. C. et al. A TALE nuclease architecture for efficient genome editing. Nat Biotechnol 29, 143-148 (2011).
5 Jinek, M. et al. A programmable dual-RNA-guided DNA endonuclease in adaptive bacterial immunity. Science 337, 816-821 (2012).
6 Cong, L. et al. Multiplex genome engineering using CRISPR/Cas systems. Science 339, 819-823 (2013).
7 Mali, P. et al. RNA-guided human genome engineering via Cas9. Science 339, 823-826 (2013).
8 Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
9 Ma, Y. et al. Targeted AID-mediated mutagenesis (TAM) enables efficient genomic diversification in mammalian cells. Nat Methods 13, 1029-1035 (2016).
10 Gaudelli, N. M. et al. Programmable base editing of A*T to G*C in genomic DNA without DNA cleavage. Nature 551, 464-471 (2017).
11 Tan, M. H. et al. Dynamic landscape and regulation of RNA editing in mammals. Nature 550, 249-254 (2017).
12 Nishikura, K. Functions and regulation of RNA editing by ADAR deaminases. Annu Rev Biochem 79, 321-349 (2010).
13 Bass, B. L. & Weintraub, H. An unwinding activity that covalently modifies its double-stranded RNA substrate. Cell 55, 1089-1098 (1988).
14 Wong. S. K., Sato, S. & Lazinski, D. W. Substrate recognition by ADAR1 and ADAR2. RNA 7, 846-858 (2001).
15 Montiel-Gonzalez, M. F., Vallecillo-Viejo, I., Yudowski, G. A. & Rosenthal, J. J. Correction of mutations within the cystic fibrosis transmembrane conductance regulator by site-directed RNA editing. Proc Natl Acad Sci USA 110, 18285-18290 (2013).
16 Sinnamon, J. R. et al. Site-directed RNA repair of endogenous Mecp2 RNA in neurons. Proc Natl Acad Sci USA 114, E9395-E9402 (2017).
17 Montiel-Gonzalez, M. F., Vallecillo-Viejo, I. C. & Rosenthal, J. J. An efficient system for selectively altering genetic information within mRNAs. Nucleic Acids Res 44, e157 (2016).
18 Hanswillemenke, A., Kuzdere, T., Vogel, P., Jekely, G. & Stafforst, T. Site-Directed RNA Editing in Vivo Can Be Triggered by the Light-Driven Assembly of an Artificial Riboprotein. J Am Chem Soc 137, 15875-15881 (2015).
19 Schneider, M. F., Wettengel, J., Hoffmann, P. C. & Stafforst, T. Optimal guideRNAs for re-directing deaminase activity of hADAR1 and hADAR2 in trans. Nucleic Acids Res 42, e87 (2014).
20 Vogel, P., Hanswillemenke, A. & Stafforst, T. Switching Protein Localization by Site-Directed RNA Editing under Control of Light. ACS synthetic biology 6, 1642-1649 (2017).
21 Vogel, P., Schneider, M. F., Wettengel, J. & Stafforst, T. Improving site-directed RNA editing in vitro and in cell culture by chemical modification of the guideRNA. Angewandte Chemie 53, 6267-6271 (2014).
22 Vogel, P. et al. Efficient and precise editing of endogenous transcripts with SNAP-tagged ADARs. Nat Methods 15, 535-538 (2018).
23 Cox, D. B. T. et al. RNA editing with CRISPR-Cas13. Science 358, 1019-1027 (2017).
24 Fukuda, M. et al. Construction of a guide-RNA for site-directed RNA mutagenesis utilising intracellular A-to-I RNA editing. Scientific reports 7, 41478 (2017).
25 Wettengel, J., Reautschnig, P., Geisler, S., Kahle, P. J. & Stafforst, T. Harnessing human ADAR2 for RNA repair—Recoding a PINK1 mutation rescues mitophagy. Nucleic Acids Res 45, 2797-2808 (2017).
26 Heep, M., Mach, P., Reautschnig, P., Wettengel, J. & Stafforst, T. Applying Human ADAR1p110 and ADAR1p150 for Site-Directed RNA Editing-G/C Substitution Stabilizes GuideRNAs against Editing. Genes (Basel) 8 (2017).
27 Katrekar, D. et al. In vivo RNA editing of point mutations via RNA-guided adenosine deaminases. Nat Methods 16, 239-242 (2019).
28 Yin, H., Kauffman, K. J. & Anderson. D. G. Delivery technologies for genome editing. Nat Rev Drug Discov 16, 387-399 (2017).
29 Platt, R. J. et al. CRISPR-Cas9 knockin mice for genome editing and cancer modeling. Cell 159, 440-455 (2014).
30 Chew, W. L. et al. A multifunctional AAV-CRISPR-Cas9 and its host response. Nat Methods 13, 868-874 (2016).
31 Teoh, P. J. et al. Aberrant hyperediting of the myeloma transcriptome by ADAR1 confers oncogenicity and is a marker of poor prognosis. Blood 132, 1304-1317 (2018).
32 Vallecillo-Viejo, I. C., Liscovitch-Brauer, N., Montiel-Gonzalez, M. F., Eisenberg, E. & Rosenthal, J. J. C. Abundant off-target edits from site-directed RNA editing can be reduced by nuclear localization of the editing enzyme. RNA biology 15, 104-114 (2018).
33 Mays, L. E. & Wilson, J. M. The complex and evolving story of T cell activation to AAV vector-encoded transgene products. Mol Ther 19, 16-27 (2011).
34 Wagner, D. L. et al. High prevalence of Streptococcus pyogenes Cas9-reactive T cells within the adult human population. Nat Med 25, 242-248 (2019).
35 Simhadri, V. L. et al. Prevalence of Pre-existing Antibodies to CRISPR-Associated Nuclease Cas9 in the USA Population. Mol Ther Methods Clin Dev 10, 105-112 (2018).
36 Charlesworth, C. T. et al. Identification of preexisting adaptive immunity to Cas9 proteins in humans. Nat Med 25, 249-254 (2019).
37 Haapaniemi, E., Botla, S., Persson, J., Schmierer, B. & Taipale, J. CRISPR-Cas9 genome editing induces a p53-mediated DNA damage response. Nat Med 24, 927-930 (2018).
38 Ihry, R. J. et al. p53 inhibits CRISPR-Cas9 engineering in human pluripotent stem cells. Nat Med 24, 939-946 (2018).

1: A method for editing a target RNA in a host cell, comprising introducing a deaminase-recruiting RNA (dRNA) into the host cell, wherein:
(1) the dRNA comprises a targeting RNA sequence that is at least partially complementary to the target RNA,
(2) the dRNA is capable of recruiting an adenosine deaminase acting on RNA (ADAR), and
(3) the dRNA is a circular RNA formed in vitro from a linear RNA.

2: The method of claim 1, wherein the linear RNA comprises a 3′ ligation sequence and a 5′ ligation sequence, optionally wherein:
(i) the 3′ ligation sequence and the 5′ ligation sequence are at least partially complementary to each other; and/or
(ii) the 3′ ligation sequence and the 5′ ligation sequence are about 20 to about 75 nucleotides in length.

3-5. (canceled)

6: The method of claim 1, wherein the linear RNA is circularized by autocatalysis of a Group I intron comprising a 5′ catalytic Group I intron fragment and a 3′ catalytic Group I intron fragment.

7: The method of claim 6, wherein the linear RNA comprises the 3′ catalytic Group I intron fragment flanking the 5′ end of a 3′ exon sequence recognizable by the 3′ catalytic Group I intron fragment, and the 5′ catalytic Group I intron fragment flanking the 3′ end of a 5′ exon sequence recognizable by the 5′ catalytic Group I intron fragment.

8. (canceled)

9: The method of claim 6, wherein said forming a circular RNA comprises:
(a) subjecting the linear RNA to a condition that activates autocatalysis of the 5′ catalytic Group I intron fragment and the 3′ catalytic Group I intron fragment to provide a circularized RNA product; and
(b) isolating the circularized RNA product, thereby providing the circular RNA.

10: The method of claim 1, wherein the linear RNA is circularized by a ligase.

11: The method of claim 6, wherein the ligase is selected from the group consisting of a T4 DNA ligase (T4 Dnl), a T4 RNA ligase 1 (T4 Rnl1), and a T4 RNA ligase 2 (T4 Rnl2).

12: The method of claim 11, wherein the linear RNA comprises a 5′ ligation sequence at the 5′ end of a nucleic acid sequence encoding the circular RNA, and a 3′ ligation sequence at the 3′ end of the nucleic acid sequence encoding the circular RNA, and wherein the 5′ ligation sequence and the 3′ ligation sequence can be ligated to each other via the ligase.

13: The method of claim 12, wherein said forming a circular RNA comprises:
(a) contacting the linear RNA with a single-stranded adaptor nucleic acid comprising from the 5′ end to the 3′ end: a first sequence complementary to the 3′ ligation sequence and a second sequence complementary to the 5′ ligation sequence, and wherein the 5′ ligation sequence and the 3′ ligation sequence hybridize to the single-stranded adaptor nucleic acid to provide a duplex nucleic acid intermediate comprising a single strand break between the 3′ end of the 5′ ligation sequence and the 5′ end of the 3′ ligation sequence;
(b) contacting the duplex nucleic acid intermediate with an RNA ligase under a condition that allows ligation of the 5′ ligation sequence to the 3′ ligation sequence to provide a circularized RNA product; and
(c) isolating the circularized RNA product, thereby providing the circular RNA.

14-15. (canceled)

16: The method of claim 1, wherein:
(i) the targeting RNA sequence is more than 50 nucleotides in length;
(ii) the targeting RNA sequence comprises a cytidine, adenosine or uridine directly opposite a target adenosine in the target RNA;
(iii) the targeting RNA sequence further comprises one or more guanosines each opposite a non-target adenosine in the target RNA;
(iv) the targeting RNA sequence comprises two or more consecutive mismatch nucleotides opposite a non-target adenosine in the target RNA;
(v) the 5′ nearest neighbor of the target adenosine in the target RNA is a nucleotide selected from the group consisting of U, C, A and G with the preference U>C≈A>G and the 3′ nearest neighbor of the target adenosine in the target RNA is a nucleotide selected from the group consisting of G, C, A and U with the preference G>C>A≈U;
(vi) the target adenosine is in a three-base motif selected from the group consisting of UAG, UAC, UAA, UAU, CAG, CAC, CAA, CAU, AAG, AAC, AAA, AAU, GAG, GAC, GAA and GAU in the target RNA; and/or
(vii) the target RNA is an RNA selected from the group consisting of a pre-messenger RNA, a messenger RNA, a ribosomal RNA, a transfer RNA, a long non-coding RNA, and a small RNA.

17: The method of claim 1, further comprising introducing one or more of the following to the host cell:
(i) an inhibitor of ADAR3;
ii a stimulator of interferon, or
(iii) a plurality of dRNAs each targeting a different target RNA.

18-21. (canceled)

22: The method of claim 16, wherein deamination of the target adenosine in the target RNA results in a missense mutation, an early stop codon, aberrant splicing, or alternative splicing in the target RNA, or reversal of a missense mutation, an early stop codon, aberrant splicing, or alternative splicing in the target RNA.

23: The method of claim 1, wherein the host cell is an eukaryotic cell.

24. (canceled)

25: A method for treating or preventing a disease or condition in an individual, the method comprising editing a target RNA associated with the disease or condition in a cell of the individual according to the method of claim 1.

26. (canceled)

27: The method of claim 25, wherein:
(i) the target RNA is TP53, and the disease or condition is cancer;
(ii) the target RNA is IDUA, and the disease or condition is Mucopolysaccharidosis type I (MPS I);
(iii) the target RNA is COL3A1, and the disease or condition is Ehlers-Danlos syndrome;
(iv) the target RNA is BMPR2, and the disease or condition is Joubert syndrome;
(v) the target RNA is FANCC, and the disease or condition is Fanconi anemia;
(vi) the target RNA is MYBPC3, and the disease or condition is primary familial hypertrophic cardiomyopathy; or
(vii) the target RNA is IL2RG, and the disease or condition is X-linked severe combined immunodeficiency.

28-33. (canceled)

34. The method of claim 1, wherein the dRNA comprises:
(1) a targeting RNA sequence that is at least partially complementary to the target RNA; and
(2) a small nucleolar RNA (snoRNA) sequence at the 3′ and/or 5′ ends of the targeting RNA sequence.

35-43. (canceled)
